(19) United States
(12) Patent Application Publication
(10) Pub. No.: US 2026/0285817 A1
(43) Pub. Date: Sep. 24, 2026

(54) CRYSTALLINE FORMS OF A 1H-1,2,3-TRIAZOLE-4-CARBOXYLIC ACID COMPOUND

(71) Applicants: CANTERO THERAPEUTICS, INC., Palo Alto, CA (US); BRIDGEBIO SERVICES INC., Palo Alto, CA (US); PORTON PHARMA SOLUTIONS LTD., Chongqing, Sichuan (CN)

(72) Inventors: JUAN HU, SHANGHAI, JIANGSU (CN); XINXU ZHANG, SHANGHAI, JIANGSU (CN); FEI SHENG, SHANGHAI, JIANGSU (CN); SHANMING JUANG, Cranbury, NJ (US); CHRISTOPHER E. HENRY, Palo Alto, CA (US)

(21) Appl. No.: 19/473,716

(22) PCT Filed: Apr. 11, 2024
(86) PCT No.: PCT/US2024/024106

§ 371 Date: Oct. 8, 2025

(30) Foreign Application Priority Data
Apr. 12, 2023 (WO) PCT/CN2023/087904

(51) Int. Cl. C07D 249/04; A61K 31/4192

(52) U.S. Cl. CPC C07D 249/04; A61K 31/4192; C07B 2200/13

(57) ABSTRACT

The present disclosure provides 1) crystalline Form A of Compound 1a or a tautomer thereof; 2) crystalline Forms D and E of Compound 1b, or a tautomer and/or hydrate thereof; and 3) co-crystalline Form C of Compound 1 and Compound 1b, or a tautomer thereof, each of which is represented by the formula: (Compound 1a), (Compound 1b), (Compound 1), or a tautomer thereof. The present disclosure also provides methods for preparing crystalline Form A of Compound 1a. Compositions including one or more of crystalline Forms A, D, E, and C and methods of treating a disease or disorder (e.g., primary hyperoxaluria (PHI), kidney and/or bladder stones, hyperoxaluria) thereof are also provided herein. Further, the present disclosure provides processes for preparing compounds (e.g., Compound 1, Compound 1b, and formula (I)—an ester of Compound 1).

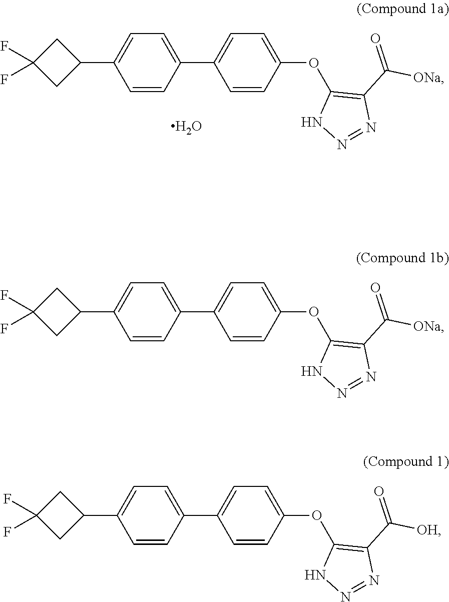

### CROSS-REFERENCES TO RELATED APPLICATIONS

This application claims priority to International Application No. PCT/CN2023/087904, filed 12 Apr. 2023, which is incorporated in its entirety by reference for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND

Kidney stone disease (KSD) has a prevalence of approximately 10% in developed countries with lifetime recurrence rates of up to 50% [Johri, et al. (2010) Nephron Clin Pract. 116: c159]. KSD patients present with hematuria and renal colic and medical treatment is essentially symptomatic. The administration of drugs to facilitate stone passage is effective for small stones (<5 mm). For bigger stones, extracorporeal sound waves or minimally invasive surgery are used to break the stone into small pieces that can more easily pass the urinary tract [Coe et al. (2005) J. Clin. Invest. 115: 2598].

Approximately 75% of kidney stones contain primarily calcium oxalate and elevated levels of urinary oxalate are found in up to 50% of KSD patients. Furthermore, increased levels of urinary oxalate increase the risk of forming kidney stones [Moe (2006) Lancet 367: 333; Sakhaee (2009) Kidney Int. 75: 585; Kaufman et al. (2008) J Am Soc Nephrol. 19: 1197]. In mammals, calcium has vital physiological roles in many processes, requiring maintenance of blood concentrations within a very narrow range that is achieved through tight regulation of intestinal absorption, uptake or release by bone, and reabsorption or elimination by the kidney. Oxalate is a metabolic end-product with no known physiological role. Oxalate is a divalent anion eliminated in urine and can combine with calcium and precipitate as insoluble calcium oxalate crystals. These crystals can aggregate as stones within the urinary tract as described above or be deposited directly in soft tissues causing direct damage and impairing organ function.

Primary hyperoxalurias (PH) are a group of rare metabolic diseases with autosomal recessive inheritance affecting the glycine/glyoxylate salvage or the hydroxyproline catabolic pathways. To date, three distinct forms of primary hyperoxaluria (primary hyperoxaluria types 1, 2, and 3) have been identified. Primary hyperoxaluria type 1 (PH1) is caused by mutation of liver-specific enzyme alanine-glyoxylate aminotransferase (AGT) and accounts for approximately 80% of all PH patients [Salido et al. (2012) Biochim Biophys Acta. 1822: 1453]. Primary hyperoxaluria type 2 (PH2) is caused by mutation of glyoxylate reductase-hydroxypyruvate reductase (GRHPR). Primary hyperoxaluria type 3 (PH3) is caused by mutation of 4-hydroxy-2-oxoglutarate aldolase (HOGA1). All three forms of PH result have in common an overproduction of oxalate. PH1 displays the most severe clinical phenotype with high penetrance, early age of onset, and rapid progression of renal damage leading to end-stage renal disease (ESRD). PH2 and PH3 display a less severe phenotype and milder clinical course.

Minor increases in urinary oxalate can produce a large effect on calcium oxalate crystal formation, and elevated levels of urinary oxalate are a major risk factor for the formation of calcium oxalate kidney stones [Pak, et al. (2004) Kidney Int. 66: 2032]. Consequently, minor reductions in urinary oxalate concentration could lower combined calcium and oxalate concentrations below saturation (the condition in which the concentrations of calcium and oxalate exceed the solubility of calcium oxalate, favoring spontaneous crystallization and precipitation or aggregation). Lowering urinary calcium and oxalate below saturation could reduce or prevent calcium oxalate stone formation and/or tissue deposition. Irrespective of urinary oxalate levels in individuals with kidney stone disease, lowering urinary oxalate (UOx) levels will decrease the likelihood of calcium oxalate crystallization and potentially lower the probability of stone formation and/or alleviate the severity of excessive calcium oxalate deposition-related conditions [Marengo et al. (2008) Nat Clin Pract Nephrol. 4: 368].

An effective drug that reduces urinary oxalate levels could be a valuable therapeutic option in the prophylaxis and treatment of conditions related to calcium oxalate. Common approaches for treatment of urolithiasis due to calcium oxalate include surgical or ureteroscopic removal of stones, extracorporeal lithotripsy, dietary management to increase fluid intake and to restrict oxalate intake, urine alkalization, thiazide diuretics, and crystallization inhibitors such as citrate, bicarbonate, and magnesium [Moe, supra]. However, none of these therapeutic approaches tackles the origin of these conditions. No drug which specifically inhibits the endogenous biosynthetic formation of oxalate is currently available for the prophylaxis and treatment of calcium oxalate deposition-related conditions.

In humans, dietary oxalate contributes 10-50% to excreted urinary oxalate [Holmes, et al. (2001) Kidney Int. 59: 270]. The majority of urinary oxalate is derived from endogenous metabolism, mainly in liver. In humans, the major precursor of oxalate is glyoxylate. Therefore, approaches to reduce the production of oxalate may inhibit the conversion of glyoxylate to oxalate or the production of glyoxylate from its precursors. In humans, glyoxylate is produced predominantly from glycolate in a reaction catalyzed by the peroxisomal liver enzyme glycolate oxidase (GO), also termed hydroxyacid oxidase 1 (HAOX1). Pharmacological inhibition of GO activity is expected to reduce endogenous oxalate production leading to a reduction in calcium oxalate levels in the urine, thus providing a targeted approach for prophylaxis and treatment of calcium oxalate stone formation and tissue deposition-related conditions. Inhibition of GO is hypothesized to be a safe therapeutic target in humans given the observation that an individual harboring a defective splice variant of human GO leading to complete inactivity (spontaneous human null) caused isolated, asymptomatic glycolic aciduria with no apparent ill effects [Frishberg, et al. (2014) J Med Genet. 51: 526]. Kinetic studies have suggested a structural similarity between the substrate binding sites in HAOX1 and LDH (Murray et al, 2008), since both enzymes can oxidize glyoxylate to oxalate (Duncan and Tipton, 1969). Although LDH catalyzes the conversion efficiently, in hyperoxaluria, the formation of oxalate might still be mostly attributed to HAOX1. The impermeability of the peroxisome to glyoxylate makes the substrate inaccessible to LDH, which is present in the cytoplasm. Glyoxylate must be transported out of the peroxisome in order to be oxidized to oxalate by LDH. High concentration of glyoxylate may have a saturating effect on transporters and the substrate for LDH may remain in the peroxisome (Jones et al, 2000). Thus, GO may be a better target for inhibition of oxalate formation in hyperoxaluria.

Compound 1, represented by the formula:

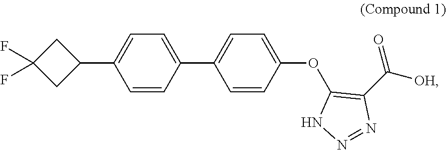 [SMILES: O=C(O)c1nn[nH]c1Oc1ccc(-c2ccc(C3CC(F)(F)C3)cc2)cc1]

or a tautomer thereof and/or a salt thereof, was first disclosed in International Application No. PCT/US2019/040690 for the treatment of Primary hyperoxaluria type 1 (PH1) and/or kidney stones. Compound 1, or a tautomer thereof and/or a salt thereof, was prepared via a Suzuki coupling reaction, as described in International Application No. PCT/US2019/040690 (referred herein as the '690 application) and International Application No. PCT/US2021/012545 (referred herein as the '545 application).

However, a crystalline form of Compound 1, a tautomer, a hydrate, and/or a pharmaceutically acceptable salt thereof, is not known. Therefore, there is a need to develop a stable crystalline form of the compound that can be stored as an active pharmaceutical ingredient (API) in the development of a drug product for the treatment of a disease or disorder (e.g., primary hyperoxaluria (PH1), kidney and/or bladder stones, hyperoxaluria).

Despite the above described processes in both the '690 and '545 applications, there remains a need for the development of more efficient and improved processes for preparing of Compound 1, a tautomer, a hydrate, and/or a pharmaceutically acceptable salt thereof. The present disclosure meets the need, in particular eliminating a Suzuki coupling reaction as the main coupling reaction to provide a compound of formula (I) (i.e., an ester of Compound 1).

### BRIEF SUMMARY

The present disclosure provides 1) crystalline Form A of Compound 1a or a tautomer thereof; 2) crystalline Forms D and E of Compound 1b, or a tautomer and/or hydrate thereof; and 3) co-crystalline Form C of Compound 1 and Compound 1b, or a tautomer thereof, each of which is represented by the formula:

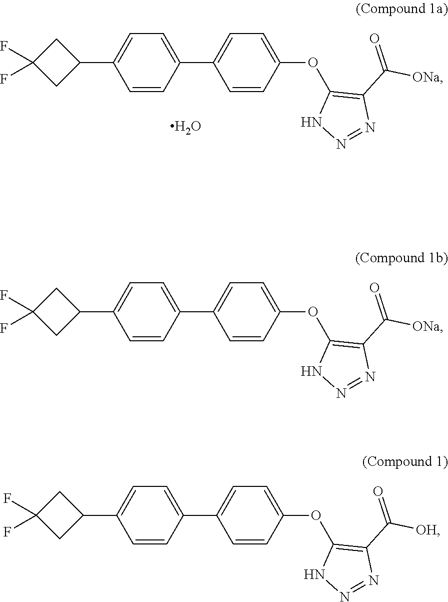

or a tautomer thereof. The present disclosure also provides methods for preparing crystalline Form A of Compound 1a. Compositions including one or more of crystalline Forms A, D, E, and C and methods of treating a disease or disorder (e.g., primary hyperoxaluria (PH1), kidney and/or bladder stones, hyperoxaluria) thereof are also provided herein. Further, the present disclosure provides processes for preparing compounds (e.g., Compound 1, Compound 1b, and formula (I) an ester of Compound 1).

In an aspect, the present disclosure provides 1) crystalline Form A of Compound 1a or a tautomer thereof; 2) crystalline Form D or E of Compound 1b, or a tautomer and/or hydrate thereof; and 3) co-crystalline Form C of Compound 1 and Compound 1b, or a tautomer thereof, wherein each of crystalline Forms A, D, E, and C is characterized by an X-ray powder diffraction (XRPD) pattern as described herein.

In an aspect, the present disclosure provides methods for preparing crystalline Form A of Compound 1a represented by the formula:

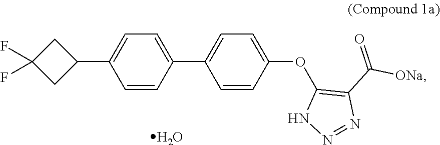

wherein Form A is described herein.

In some embodiments, the present disclosure provides a method for preparing crystalline Form A of Compound 1a, the method including:
a) providing a first mixture including:
   i) Compound 1 represented by the formula:

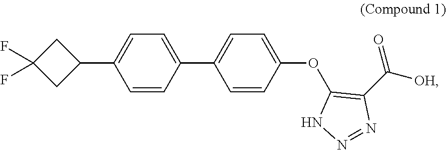 [SMILES: O=C(O)c1nn[nH]c1Oc1ccc(-c2ccc(C3CC(F)(F)C3)cc2)cc1]

a tautomer, a sodium salt, a hydrate, or a combination thereof; and
ii) a solvent mixture including tetrahydrofuran (THF) and water;
b) stirring the first mixture at a temperature of from about 45° C. to about 60° C. to form a second mixture;
c) filtering the second mixture of step b) to provide a third mixture including Compound 1b represented by the formula:

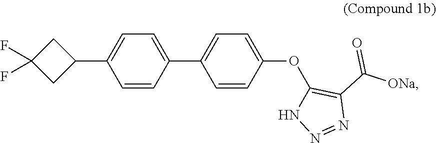

or a tautomer thereof;
d) cooling the third mixture to a temperature of less than 10° C. to form a slurry; and
e) isolating a solid from the slurry to provide the crystalline Form A.

In another aspect, the present disclosure provides a pharmaceutical composition including one or more of crystalline Forms A, D, E, and C, as described herein; and a pharmaceutically acceptable excipient. In some embodiments, the pharmaceutical composition includes crystalline Form A of Compound 1a or a tautomer thereof and a pharmaceutically acceptable excipient, wherein Form A is described herein.

In still another aspect, the present disclosure provides a method for treating primary hyperoxaluria (PH1), including administering to a subject in need thereof a therapeutically effective amount of Compound 1 or a tautomer thereof including one or more of crystalline Forms A, D, E, and C, as described herein, or a pharmaceutical composition as described herein. In some embodiments, the method for treating PH1 includes administering Compound 1a or a tautomer thereof including crystalline Form A, and a pharmaceutically acceptable salt thereof; or a pharmaceutical composition including Form A to a subject in need thereof.

In another aspect, the present disclosure provides a method for treating kidney and/or bladder stones, the method including administering to a subject in need thereof a therapeutically effective amount of Compound 1 or a tautomer thereof including one or more of crystalline Forms A, D, E, and C, as described herein, or a pharmaceutical composition as described herein. In some embodiments, the method for treating kidney and/or bladder stones includes administering Compound 1a or a tautomer thereof including crystalline Form A, and a pharmaceutically acceptable salt thereof; or a pharmaceutical composition including Form A to a subject in need thereof.

In yet another aspect, the present disclosure provides a method of treating hyperoxaluria in a subject in need thereof, the method including administering to the subject a therapeutically effective amount of Compound 1, a tautomer, a pharmaceutically acceptable salt, a hydrate, or a combination thereof, including one or more of crystalline Forms A, D, E, and C, as described herein, or a pharmaceutical composition including one or more of crystalline Forms A, D, E, and C, as described herein, wherein the therapeutically effective amount reduces a maximal urinary oxalate (uOxalate) excretion: i) to less than about 0.46 millimoles per 1.73 square meters per 24 hours (mmol/1.73 m²/24 hr), or ii) by at least about 50% relative to a uOxalate excretion prior to the treating.

In some embodiments, provided herein is a method of treating hyperoxaluria in a subject in need thereof, the method including administering to the subject a therapeutically effective amount of Compound 1a or a tautomer thereof including crystalline Form A as described herein or a pharmaceutical composition including Form A as described herein, wherein the therapeutically effective amount reduces a maximal urinary oxalate (uOxalate) excretion: i) to less than about 0.46 millimoles per 1.73 square meters per 24 hours (mmol/1.73 m²/24 hr)), or ii) by at least about 50% relative to a uOxalate excretion prior to the treating.

In another aspect, the present disclosure provides a method of treating hyperoxaluria in a subject in need thereof, the method including administering to the subject a therapeutically effective amount of Compound 1, a tautomer, a pharmaceutically acceptable salt, a hydrate, or a combination thereof, including one or more of crystalline Forms A, D, E, and C, as described herein, or a pharmaceutical composition including one or more of crystalline Forms A, D, E, and C, as described herein, wherein the therapeutically effective amount is a total daily dosage of about 20 milligrams (mg) to about 2,500 mg.

In some embodiments, provided herein is a method of treating hyperoxaluria in a subject in need thereof, the method including administering to the subject a therapeutically effective amount of Compound 1a or a tautomer thereof including crystalline Form A as described herein or a pharmaceutical composition including Form A as described herein, wherein the therapeutically effective amount is a total daily dosage of about 20 mg to 2,500 mg.

In another aspect, the present disclosure provides a method of treating kidney stones in a subject in need thereof, the method including administering to the subject a therapeutically effective amount of Compound 1, a tautomer, a pharmaceutically acceptable salt, a hydrate, or a combination thereof, including one or more of crystalline Forms A, D, E, and C, as described herein, or a pharmaceutical composition including one or more of crystalline Forms A, D, E, and C, as described herein, wherein said therapeutically effective amount is a total daily dosage of about 20 mg to about 2,500 mg.

In some embodiments, provided herein is a method of treating kidney stones in a subject in need thereof, the method including administering to the subject a therapeutically effective amount of Compound 1a or a tautomer thereof including crystalline Form A as described herein or a pharmaceutical composition including Form A as described herein, wherein said therapeutically effective amount is a total daily dosage of about 20 mg to about 2,500 mg.

In yet another aspect, the present disclosure provides a method of preventing the development, worsening, or recurrence of kidney stones in a subject in need thereof, the method including administering to the subject a therapeutically effective amount of Compound 1, a tautomer, a pharmaceutically acceptable salt, a hydrate, or a combination thereof, including one or more of crystalline Forms A, D, E, and C, as described herein, or a pharmaceutical composition including one or more of crystalline Forms A, D, E, and C, as described herein, wherein said therapeutically effective amount is a total daily dosage of about 20 mg to about 2,500 mg.

In some embodiments, provided herein is a method of preventing the development, worsening, or recurrence of kidney stones in a subject in need thereof, the method including administering to the subject a therapeutically effective amount of Compound 1a or a tautomer thereof including crystalline Form A as described herein or a pharmaceutical composition including Form A as described herein, wherein said therapeutically effective amount is a total daily dosage of about 20 mg to about 2,500 mg.

In yet another aspect, the present disclosure provides a method of reducing the size of kidney stones in a subject in need thereof, the method comprising administering to the subject a therapeutically effective amount of Compound 1, a tautomer, a pharmaceutically acceptable salt, a hydrate, or a combination thereof, including one or more of crystalline Forms A, D, E, and C, as described herein, or a pharmaceutical composition including one or more of crystalline Forms A, D, E, and C, as described herein, wherein said therapeutically effective amount is a total daily dosage of about 20 mg to about 2,500 mg.

In some embodiments, provided herein is a method of reducing the size of kidney stones in a subject in need thereof, the method comprising administering to the subject a therapeutically effective amount of Compound 1a or a tautomer thereof including crystalline Form A as described herein or a pharmaceutical composition including Form A as described herein, wherein said therapeutically effective amount is a total daily dosage of about 20 mg to about 2,500 mg.

In an aspect, the present disclosure provides a process for preparing a compound represented by formula (I):

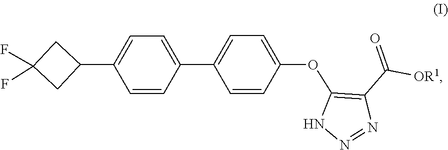

or a tautomer and/or a salt thereof via Steps a) and b). The process includes:
a) combining a compound of formula (IV):

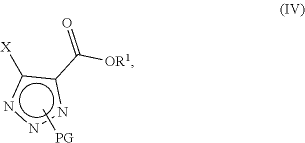

or a salt thereof, a compound of formula (III):

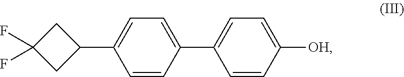 [SMILES: Oc1ccc(-c2ccc(C3CC(F)(F)C3)cc2)cc1]

or a salt thereof, and a first base in a first solvent to form a compound of formula (II):

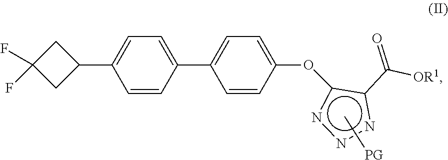

or a salt thereof; and
b) removing the PG group of the compound of formula (II) or the salt thereof to provide a compound of formula (I):

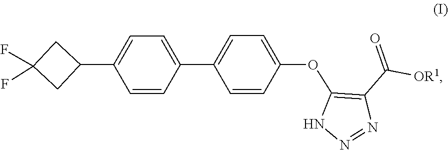

or a tautomer and/or a salt thereof,
wherein:
R¹ is C₁₋₆ alkyl;
X in formula (IV) is halogen or a sulfonate;

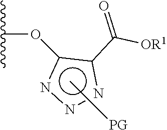

in formula (IV) or (II) is represented by the formula:

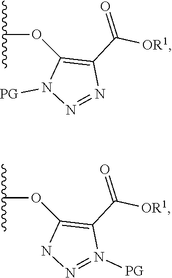

or a mixture thereof; and
PG is an amine-protecting group.

In another aspect, the present disclosure provides a process for preparing Compound 1 represented by the formula:

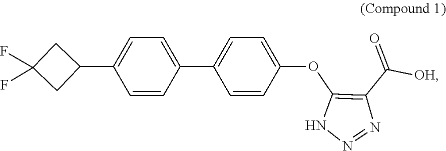 [SMILES: O=C(O)c1nn[nH]c1Oc1ccc(-c2ccc(C3CC(F)(F)C3)cc2)cc1]

a tautomer, a sodium salt, a hydrate, or a combination thereof, via steps a) to d). The process includes:
a) combining a compound of formula (IVa):

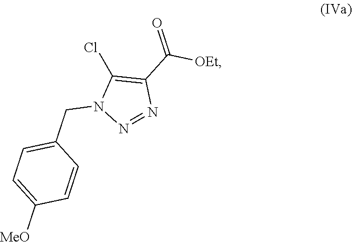 [SMILES: CCOC(=O)c1nnn(Cc2ccc(OC)cc2)c1Cl]

or a salt thereof, a compound of formula (III):

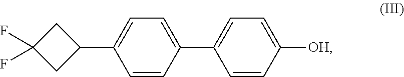 [SMILES: Oc1ccc(-c2ccc(C3CC(F)(F)C3)cc2)cc1]

or a salt thereof, and a first base in a first solvent to form a compound of formula (IIa):

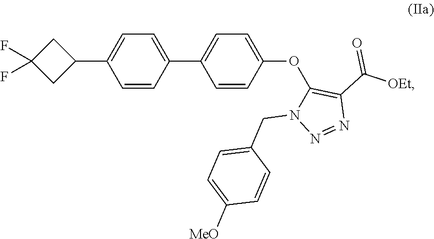 [SMILES: CCOC(=O)c1nnn(Cc2ccc(OC)cc2)c1Oc1ccc(-c2ccc(C3CC(F)(F)C3)cc2)cc1]

or a salt thereof, wherein the first base is 1,8-diazabicyclo[5.4.0]undec-7-ene (DBU) and the first solvent includes N,N-dimethylacetamide (DMAC); or the first base is cesium carbonate and the first solvent includes N-methylpyrrolidone (NMP);
b) treating the compound of formula (IIa) or the salt thereof, with trifluoroacetic acid and anisole to provide a compound of formula (Ia-1):

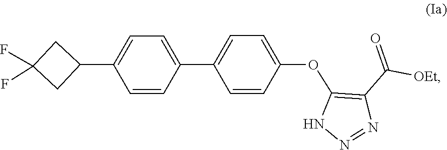 [SMILES: CCOC(=O)c1nn[nH]c1Oc1ccc(-c2ccc(C3CC(F)(F)C3)cc2)cc1]

or a tautomer and/or a salt thereof:
c) saponifying the compound of formula (Ia), or the tautomer and/or the salt thereof, with an aqueous solution of sodium hydroxide; and
d) acidifying with an aqueous solution of HCl to provide Compound 1, the tautomer, the sodium salt, the hydrate, or the combination thereof.

### BRIEF DESCRIPTION OF THE DRAWINGS

FIG. 1 shows an X-ray powder diffraction pattern of crystalline Form A of Compound 1a.

FIG. 2 shows a differential scanning calorimetry (DSC) thermogram of crystalline Form A of Compound 1a.

FIG. 3 shows a thermal gravimetric analysis (TGA) thermogram of crystalline Form A of Compound 1a.

FIG. 4 shows a polarized light microscopy (PLM) of crystalline Form A of Compound 1a.

FIG. 5 shows a dynamic vapor sorption profile of crystalline Form A of Compound 1a.

FIG. 6 shows an X-ray powder diffraction pattern of crystalline Form D of Compound 1b.

FIG. 7 shows a differential scanning calorimetry (DSC) thermogram of crystalline Form D of Compound 1b.

FIG. 8 shows a thermal gravimetric analysis (TGA) thermogram of crystalline Form D of Compound 1b.

FIG. 9 shows a polarized light microscopy (PLM) image of crystalline Form D of Compound 1b.

FIG. 10 shows an X-ray powder diffraction pattern of crystalline Form E of Compound 1b.

FIG. 11 shows a differential scanning calorimetry (DSC) thermogram of crystalline Form E of Compound 1b.

FIG. 12 shows a thermal gravimetric analysis (TGA) thermogram of crystalline Form E of Compound 1b.

FIG. 13 shows an X-ray powder diffraction pattern of co-crystalline Form C of Compounds 1 and 1b.

FIG. 14 shows a differential scanning calorimetry (DSC) thermogram of co-crystalline Form C of Compounds 1 and 1b.

FIG. 15 shows a thermal gravimetric analysis (TGA) thermogram of co-crystalline Form C of Compounds 1 and 1b.

FIG. 16 shows an X-ray powder diffraction pattern post-heating of crystalline Form A of Compound 1a.

FIG. 17 shows an X-ray powder diffraction pattern of a starting material including Compounds 1, 1a, and 1b.

FIG. 18 shows both a differential scanning calorimetry (DSC) and a thermal gravimetric analysis (TGA) thermograms of a starting material including Compounds 1, 1a, and 1b.

FIG. 19 shows the synthetic scheme for preparing 4′-(3,3-Difluorocyclobutyl)-[1,1′-biphenyl]-4-ol (the compound of formula (III)).

FIG. 20 shows the synthetic scheme for preparing sodium 5-((4′-(3,3-difluorocyclobutyl)-[1,1′-biphenyl]-4-yl)oxy)-1H-1,2,3-triazole-4-carboxylate (Compound 1b).

### DETAILED DESCRIPTION

### I. General

The present disclosure provides 1) crystalline Form A of Compound 1a or a tautomer thereof; 2) crystalline Forms D and E of Compound 1b, or a tautomer and/or hydrate thereof; and 3) co-crystalline Form C of Compound 1 and Compound 1b, or a tautomer thereof, each of which is represented by the formula:

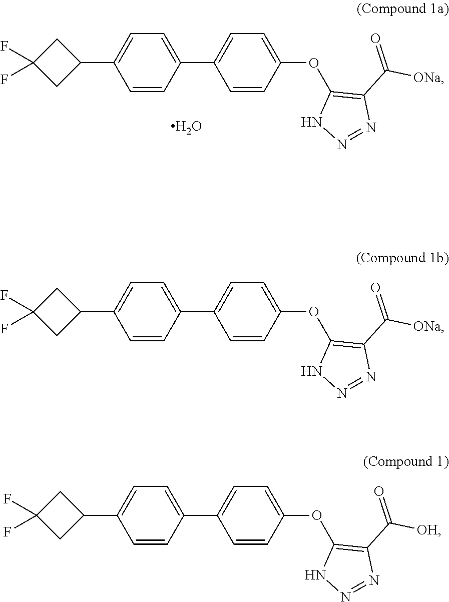

or a tautomer thereof. The present disclosure also provides methods for preparing crystalline Form A of Compound 1a. Compositions including one or more of crystalline Forms A, D, E, and C and methods of treating a disease or disorder (e.g., primary hyperoxaluria (PH1), kidney and/or bladder stones, hyperoxaluria) thereof are also provided herein. Further, the present disclosure provides processes for preparing compounds (e.g., Compound 1, Compound 1b, and formula (I) an ester of Compound 1).

### II. Definitions

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The terms “a” or “an,” as used in herein means one or more.

“Comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

“Compound 1” refers to the chemical 5-((4′-(3,3-difluorocyclobutyl)-[1,1′-biphenyl]-4-yl)oxy)-1H-1,2,3-triazole-4-carboxylic acid, having the formula:

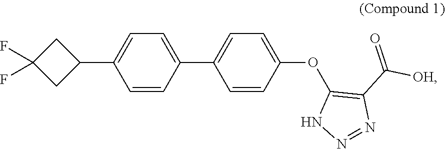 [SMILES: O=C(O)c1nn[nH]c1Oc1ccc(-c2ccc(C3CC(F)(F)C3)cc2)cc1]

a tautomer, a pharmaceutically acceptable salt, hydrate, solvate, or a combination thereof. Compound 1 and related compounds are disclosed in International Patent Application No. PCT/US2019/040690, which is herein incorporated by reference in its entirety.

Unless specifically indicated otherwise, “an acid form” of Compound 1 refers to Compound 1 represented by the formula:

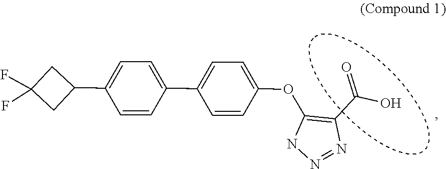

or a tautomer thereof, wherein the group (circled) is a carboxylic acid.

“Compound 1a” refers to the chemical sodium 5-((4′-(3,3-difluorocyclobutyl)-[1,1′-biphenyl]-4-yl)oxy)-1H-1,2,3-triazole-4-carboxylate monohydrate, represented by the formula:

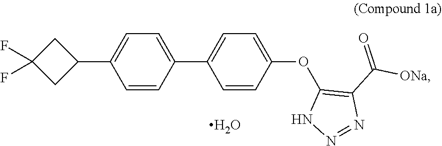

or a tautomer thereof.

“Compound 1b” refers to the chemical sodium 5-((4′-(3,3-difluorocyclobutyl)-[1,1′-biphenyl]-4-yl)oxy)-1H-1,2,3-triazole-4-carboxylate, represented by the formula:

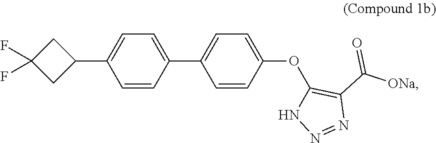

or a tautomer and/or hydrate thereof, provided that Compound 1b is other than Compound 1a. In some embodiments, Compound 1b is in an anhydrate form. In some embodiments, Compound 1b is in a hemihydrate form.

It will be apparent to one skilled in the art that Compound 1, 1a, and 1b of this disclosure may exist in tautomeric forms, all such tautomeric forms of Compound 1, 1a, and 1b being within the scope of the disclosure. Tautomer refers to one of two or more structural isomers which exist in equilibrium and which are readily converted from one isomeric form to another. Generally, this interconversion is sufficiently fast so that an individual tautomer is not isolated in the absence of another tautomer. For example, Compound 1 of the following formulae can exist in equilibrium:

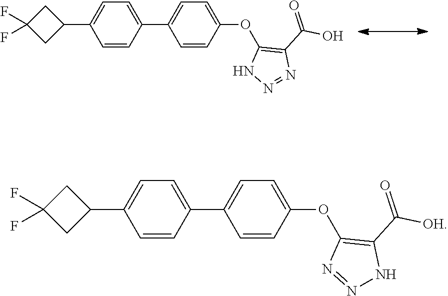 [SMILES: O=C(O)c1nn[nH]c1Oc1ccc(-c2ccc(C3CC(F)(F)C3)cc2)cc1 ; O=C(O)c1[nH]nnc1Oc1ccc(-c2ccc(C3CC(F)(F)C3)cc2)cc1]

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 1 milligram (mg) to 3 mg,” which is intended to include 1 mg, 3 mg, and everything in between to any number of significant figures (e.g., 1.255 mg, 2.1 mg, 2.9999 mg, etc.).

“About” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

“Substantially free” refers to an amount of 10% or less of another form or impurity, preferably 8%, 5%, 4%, 3%, 2%, 1%, 0.5%, or less of another form or impurity. “Crystalline Form A is substantially free of other crystalline or amorphous forms of Compound 1, 1a, and/or 1b” refers to an amount of 10% or less of the other crystalline or amorphous forms of Compound 1, 1a, and/or 1b (e.g., Form D, E, C, amorphous forms, or a combination thereof), preferably 8%, 5%, 4%, 3%, 2%, 1%, 0.5%, or less of the other crystalline or amorphous forms of Compound 1, 1a, and/or 1b.

“Crystalline form” refers to a solid form of a compound wherein the constituent molecules are packed in a regularly ordered, repeating pattern. A crystalline form can include triclinic, monoclinic, orthorhombic, tetragonal, trigonal, hexagonal, and cubic crystal geometries. A crystalline form can include one or more regions, i.e., grains, with distinct crystal boundaries. A crystalline solid can include two or more crystal geometries.

“Amorphous form” refers to a solid form of a compound having no definite crystal structure, i.e., lacking a regularly ordered, repeating pattern of constituent molecules.

“Solvate” refers to a compound provided herein or a salt thereof, that further includes a stoichiometric or non-stoichiometric amount of solvent bound by non-covalent intermolecular forces.

“Hydrate” refers to a compound that is complexed to a water molecule. The compounds of the present disclosure can be complexed with 1% water molecule or from 1 to 10 water molecules.

“Salt” refers to acid or base salts of the compounds of the present disclosure. Illustrative examples of pharmaceutically acceptable acid addition salts are mineral acid (hydrochloric acid, hydrobromic acid, phosphoric acid, and the like) salts and organic acid (acetic acid, propionic acid, glutamic acid, citric acid and the like) salts. Examples of pharmaceutically acceptable base addition salts include sodium, potassium, calcium, ammonium, organic amino, or magnesium salt, or a similar salt. It is understood that the pharmaceutically acceptable salts are non-toxic. Additional information on suitable pharmaceutically acceptable salts can be found in Remington's Pharmaceutical Sciences, 23rd Edition, 2020, which is incorporated herein by reference.

“Pharmaceutically acceptable” refers to those compounds (salts, hydrates, solvates, conformational isomers, tautomers, etc.) which are suitable for use in contact with the tissues of patients without undue toxicity, irritation, and allergic response, are commensurate with a reasonable benefit/risk ratio, and are effective for their intended use. The compounds disclosed herein can exist as pharmaceutically acceptable salts, as defined and described herein.

XRPD patterns can be indexed in the absence of suitable single crystals for structure elucidation [McClurg, Richard B.; Smit, Jared P. X-ray Powder Diffraction Pattern Indexing for Pharmaceutical Applications. Pharm. Tech. Europe, January 2013; and X′Pert High Score Plus 2.2a (2.2.1)]. Indexing is the process of determining the size and shape of the crystallographic unit cell given the peak positions in a diffraction pattern. The term gets its name from the assignment of Miller index labels to individual peaks. XRPD indexing serves several purposes. If all of the peaks in a pattern are indexed by a single unit cell, this is strong evidence that the sample contains a single crystalline phase. Given the indexing solution, the unit cell volume may be calculated directly. Indexing is also a robust description of a crystalline form and provides a concise summary of all available peak positions for that phase at a particular thermodynamic state point.

“Crude” refers to a mixture including a desired compound (e.g., Compound 1a or 1b) and at least one other species (e.g., a solvent, a reagent such as an acid or base, a starting material, or a byproduct of a reaction giving rise to the desired compound).

Unless specifically indicated otherwise, “purity %” or “purity area %” (e.g., 95% or 95 area %) refers to a purity of a compound (e.g., Compound 1, 1a, or 1b) in the area under curve (AUC) determined by a HPLC method.

“First mixture”, “second mixture”, and so on refer to a mixture as described in embodiments of the present disclosure. The mixture naming conventions are used solely for the purpose of clarity in steps of the process as described herein and they are not required to be in a numerical order. Some mixtures may be absent in selected embodiments of the present disclosure as described herein. One skilled in the art will understand the meaning of these mixture naming conventions (e.g., ‘first mixture’, ‘second mixture’) within the context of the term's use in the embodiments and claims herein.

“First slurry”, “second slurry”, and so on refer to a slurry as described in embodiments of the present disclosure. The slurry naming conventions are used solely for the purpose of clarity in steps of the process as described herein and they are not required to be in a numerical order. Some slurries may be absent in selected embodiments of the present disclosure as described herein. One skilled in the art will understand the meaning of these slurry naming conventions (e.g., ‘first slurry’, ‘second slurry’) within the context of the term's use in the embodiments and claims herein.

“First temperature”, “second temperature”, and so on refer to a temperature as described in embodiments of the present disclosure. The temperature naming conventions are used solely for the purpose of clarity in steps of the process as described herein and they are not required to be in a numerical order. One skilled in the art will understand the meaning of these temperature naming conventions (e.g., ‘first temperature’, ‘second temperature’) within the context of the term's use in the embodiments and claims herein.

“Precipitating” refers to the process of causing a compound in a solution to coalesce into a solid form of the substance (i.e., a precipitate). The entirety of a compound in a solution, or any fraction thereof, can be caused to precipitate. The solid form of the substance can be amorphous or crystalline.

“Isolating” refers to the process of isolating at least a portion of a first substance (e.g., a precipitate) from a mixture including the substance and at least one additional substance. In some instances, the isolated substance is substantially free at least one of the additional substances present in the original mixture.

“Alkyl” refers to a straight or branched, saturated, aliphatic radical having the number of carbon atoms indicated (i.e., C₁₋₆ means one to six carbons). Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc.

“Halogen” refers to fluorine, chlorine, bromine and iodine.

“Sulfonate” refers to an —SO₃R group where R group can be halo (e.g., —F), alkyl (e.g., methyl or ethyl), haloalkyl (e.g., trifluoromethyl), aryl (e.g., phenyl, tosyl, p-fluorophenyl, or p-nitrophenyl), heteroaryl (e.g., imidazolyl). Exemplary sulfonate groups include, but are not limited to, fluorosulfonate, methanesulfonate (OMs), trifluoromethanesulfonate (OTf), p-toluenesulfonate (OTs), p-fluorobenzenesulfonate, p-nitrophenylsulfonate (nosylate), and imidazole-1-sulfonate (imidazylate).

“OMs” refers to methanesulfonate; “—OTs” refers to p-toluenesulfonate; and “—OTf” refers to trifluoromethanesulfonate.

Bases useful in the present disclosure include organic bases and inorganic bases. Exemplary organic bases include amines, alkali carboxylates, and alkali alkoxides, as defined herein. Exemplary inorganic bases include alkali bicarbonates, alkali carbonates, alkali phosphates tribasic, and alkali hydroxides, as defined herein. Amines useful in the present disclosure as bases include tertiary amines, aromatic amine bases, and amidine-based compounds, as defined herein.

“First base”, “second base”, and so on refer to a base as defined above and described in embodiments of the present invention. The base naming conventions are used solely for the purpose of clarity in relevant steps of the process as described herein and they are not required to be in a numerical order. Some bases may be absent in selected embodiments of the present invention as described herein. One skilled in the art will understand the meaning of these base naming conventions (‘first base’, ‘second base’) within the context of the term's use in the embodiments and claims herein.

“Amine” refers to a compound having formula N(R)₃ where the R groups can be hydrogen, alkyl, aryl, or heteroalkyl, among others. The R groups can be the same or different. For example, the amines can be primary amine (two R is each hydrogen), secondary amine (one R is hydrogen), and tertiary amine (each R is other than hydrogen). In some embodiments, the secondary amine is a cyclic amine where two R groups bond to the nitrogen atom form a 5-6 membered heterocyclic ring. Non-limiting examples of cyclic amines include pyrrolidine, piperidine, and morpholine.

“Tertiary amine” refers to a compound having formula N(R)₃ wherein the R groups can be alkyl, aryl, heteroalkyl, heteroaryl, among others, or two R groups together form a N-linked heterocycloalkyl. The R groups can be the same or different. Non-limiting examples of tertiary amines include triethylamine, tri-n-butylamine, N,N-diisopropylethylamine, N-methylpyrrolidine, N-methylmorpholine, dimethylaniline, diethylaniline, 1,8-bis(dimethylamino)naphthalene, quinuclidine, and 1,4-diazabicylo[2.2.2]-octane (DABCO).

“Aromatic amine base” refers to a N-containing 5- to 10-membered heteroaryl compound or a tertiary amine having formula N(R)₃ wherein at least one R group is an aryl or heteroaryl. Aromatic amine bases useful in the present application include, but are not limited to, pyridine, lutidines (e.g., 2,6-lutidine, 3,5-lutidine, and 2,3-lutidine), collidines (e.g., 2,3,4-collidine, 2,3,5-collidine, 2,3,6-collidine, 2,4,5-collidine, 2,4,6-collidine, and 3,4,5-collidine), 4-dimethylaminopyridine, imidazole, 1-methylimidazole, dimethylaniline, and diethylaniline.

“Amidine-based compounds” herein refers to a class of chemical compounds that include, but are not limited to, 1,8-diazabicyclo[5.4.0]undec-7-ene (DBU) and 1,5-diazabicyclo[4.3.0]non-5-en (DBN).

“Alkali carboxylate” refers to a class of chemical compounds which are composed of an alkali metal cation and the carboxylate anion (RC(O)O—) where the R group can be alkyl or aryl. Alkali carboxylates useful in the present include, but are not limited to, lithium acetate (LiOC(O)CH₃), sodium acetate (NaOC(O)CH₃), potassium acetate (KOC(O)CH₃), cesium acetate (CsOC(O)CH₃), and potassium trimethylacetate (KOC(O)C(CH₃)₃).

“Alkali bicarbonate” refers to a class of chemical compounds which are composed of an alkali metal cation and the carbonate anion (HCO₃⁻). Alkali carbonates useful in the present disclosure include lithium bicarbonate (LiHCO₃), sodium bicarbonate (NaHCO₃), potassium bicarbonate (KHCO₃), and cesium bicarbonate (CsHCO₃).

“Alkali carbonate” refers to a class of chemical compounds which are composed of an alkali metal cation and the carbonate anion (CO₃²⁻). Alkali carbonates useful in the present disclosure include lithium carbonate (Li₂CO₃), sodium carbonate (Na₂CO₃), potassium carbonate (K₂CO₃), and cesium carbonate (Cs₂CO₃).

“Alkali phosphate tribasic” refers to a class of chemical compounds which are composed of an alkali metal cation and the phosphate anion (PO₄³⁻). Alkali phosphates tribasic useful in the present disclosure include sodium phosphate tribasic (Na₃PO₄) and potassium phosphate tribasic (K₃PO₄).

“Alkali hydroxide” refers a class of chemical compounds which are composed of an alkali metal cation and the hydroxide anion (OH⁻). Alkali hydroxides useful in the present disclosure include LiOH, NaOH, KOH, and CsOH.

“Acid” refers to a compound that is capable of donating a proton (H⁺) under the Bronsted-Lowry definition, or is an electron pair acceptor under the Lewis definition. Acids useful in the present disclosure include, but are not limited to, fluorinated carboxylic acids (trifluoroacetic acid), sulfonic acids and mineral acids, as defined herein. Mineral acids are inorganic acids such as hydrogen halides (hydrofluoric acid, hydrochloric acid, hydrobromic acid, etc.), as well as sulfuric acid, nitric acid, and phosphoric acid. Sulfonic acids include methanesulfonic acid, benzenesulfonic acid, p-toluenesulfonic acid, triflouromethanesulfonic acid, among others.

“A protecting group” refers to a compound that renders a functional group unreactive to a particular set of reaction conditions, but that is then removable in a later synthetic step so as to restore the functional group to its original state. Such protecting groups are well known to one of ordinary skill in the art and include compounds that are disclosed in “Protective Groups in Organic Synthesis”, 4th edition, T. W. Greene and P. G. M. Wuts, John Wiley & Sons, New York, 2006, which is incorporated herein by reference in its entirety.

“An amine protecting group” refers a protecting group that is used to protect one of nitrogen atoms in 1,2,3-triazole group. The amine protecting groups useful in the present disclosure include, but are not limited to, a benzyl (Bn) group, 2-(trimethylsilyl)ethoxymethyl, p-methoxybenzyl (PMB), 2,4-dimethoxybenzyl group (DMB), 1-(2,4-dinethoxyphcenyl)ethyil, 3,4-dimethoxybenzyl (DMPB), p-methoxyphenyl (PMP), Tosyl (Ts) group, and other sulfonamides (Nosyl & Nps) groups.

“Deprotecting” refers to remove the protecting group as defined above with one or more chemicals or agents so that the functional group is restored to its original state.

“Transferring agent” refers to a chemical agent added to a reaction mixture during the deprotecting step in order to efficiently remove the amine protecting group, such as p-methoxybenzyl (PMB), 2,4-dimethoxybenzyl group (DMB), 1-(2,4-dimethoxyphenyl)ethyl, or 3,4-dimethoxybenzyl (DMPB). Transferring agents useful in the present disclosure include, but are not limited to, anisole or the like. It is believed that anisole is acting as a transfer reagent wherein the PMB protecting group or the like is transferred from an amine to the para position of anisole.

“Catalyst” refers to a substance that increases the rate of a chemical reaction by reducing the activation energy, but which is left unchanged by the reaction.

“Palladium catalyst” refers to a compound that is composed of palladium that can be neutral or positively charged, e.g., Pd⁰ or Pd². Palladium catalyst includes, but is not limited to, Pd(acac)₂, [Pd(allyl)Cl]₂, Pd(CH₃CN)₂Cl₂, Pd(dba)₂, Pd(CH₃COO)₂, Pd₂(dba)₃, Pd₂(dba)₃·CHCl₃, Pd(PPh₃)₄, Pd(OAc)₂, Pd(PCy₃)₂Cl₂, Pd(PPh₃)₂Cl₂, Pd[P(o-tol)₃]₂Cl₂, Pd(amphos)Cl₂, Pd(dppf)Cl₂, Pd(dppf)Cl₂·CH₂Cl₂, Pd(dtbpf)Cl₂, Pd(CH₃CN)₄(BF₄)₂, PdCl₂, XPhos-Pd-G3, Pd-PEPPSI™-IPr, Pd—PEPPSI™-SIPr, or Pd-PEPPSI™-IPent.

“Contacting” refers to the process of bringing into contact at least two distinct species such that they can react. It should be appreciated, however, the resulting reaction product can be produced directly from a reaction between the added reagents or from an intermediate from one or more of the added reagents which can be produced in the reaction mixture.

“Solvent” refers to a substance, such as a liquid, capable of dissolving a solute. Solvents can be polar or non-polar, protic or aprotic. Polar solvents typically have a dielectric constant greater than about 5 or a dipole moment above about 1.0, and non-polar solvents have a dielectric constant below about 5 or a dipole moment below about 1.0. Protic solvents are characterized by having a proton available for removal, such as by having a hydroxy or carboxy group. Aprotic solvents lack such a group. Representative polar protic solvents include alcohols (methanol, ethanol, propanol, isopropanol, etc.), acids (formic acid, acetic acid, etc.) and water. Representative polar aprotic solvents include dichloromethane, chloroform, tetrahydrofuran, methyltetrahydrofuran, diethyl ether, 1,4-dioxane, acetone, ethyl acetate, dimethylformamide, acetonitrile, dimethyl sulfoxide, and N-methylpyrrolidone. Representative non-polar solvents include alkanes (pentanes, hexanes, etc.), cycloalkanes (cyclopentane, cyclohexane, etc.), benzene, and toluene. Other solvents are useful in the present disclosure.

“Aprotic solvent” refers to solvents that lack an acidic hydrogen. Consequently, they are not hydrogen bond donors. Common characteristics of aprotic solvents are solvents that can accept hydrogen bonds, solvents do not have acidic hydrogen, and solvents dissolve salts. Examples of aprotic solvents include, but are not limited to, N-methylpyrrolidone (NMP), tetrahydrofuran (THF), 2-methyl tetrahydrofuran (MeTHF), ethyl acetate (EtOAc), acetone, dimethylformamide (DMF), acetonitrile (MeCN), dimethyl sulfoxide (DMSO), propylene carbonate (PC), and hexamethylphosphoramide (HMPA).

Solvents can also be grouped based on their chemical structures, for example, ethers (e.g., diethyl ether, methyl tert-butyl ether, tetrahydrofuran, 2-methyltetrahydrofuran, 1,4-dioxane, etc.), ketones (e.g., acetone, methyl isobutyl ketone, etc.), esters (ethyl acetate, butyl acetate, isobutyl acetate, etc.), aromatic solvents (e.g., benzene, toluene, xylenes, etc.), chlorinated solvents (e.g., dichloromethane, 1,2-dichloroethane, etc.), hydrocarbons (n-heptane, hexanes, cyclohexane, methylcyclohexane, etc.), alcohols (methanol, ethanol, propanol, isopropanol, etc.), or acids (e.g., formic acid, acetic acid, etc.).

“First solvent”, “second solvent”, and so on refer to a solvent as defined above and described in embodiments of the present invention. The solvent naming conventions are used solely for the purpose of clarity in steps of the process as described herein and they are not required to be in a numerical order. Some solvents may be absent in selected embodiments of the present invention as described herein. One skilled in the art will understand the meaning of these solvent naming conventions (e.g., ‘first solvent’, ‘second solvent’) within the context of the term's use in the embodiments and claims herein.

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, glidants, disintegrants, surfactants, lubricants, coatings, sweeteners, flavors, and colors. One of skill in the art will recognize that other pharmaceutical excipients are useful in the present disclosure.

“Tablet” refers to solid pharmaceutical formulations with and without a coating. The term “tablet” also refers to tablets having one, two, three or even more layers, wherein each of the before mentioned types of tablets may be without or with one or more coatings. In some embodiments, tablets of the present disclosure can be prepared by roller compaction or other suitable means known in the art. The term “tablet” also comprises mini, melt, chewable, effervescent, and orally disintegrating tablets. Tablets include Compound 1 or Compound 1a and one or more pharmaceutical excipients (e.g., fillers, binders, glidants, disintegrants, surfactants, binders, lubricants, and the like). Optionally, a coating agent can be also included. For the purposes of calculating percent weight of the tablet formulation, the amount of coating agent is not included in the calculation. That is, the percent weights reported herein are of the uncoated tablet.

“Primary hyperoxaluria, type I” and “PH1” are interchangeable and refer to a condition caused by the deficiency of alanine:glyoxylate aminotransferase (AGT), a liver enzyme. This deficiency causes impaired glyoxylate metabolism in the liver and an ultimate increase in oxalate synthesis, contributing to the formation of calcium oxalate kidney stones.

“Kidney stone” and/or “bladder stone” refer to a small, solid particle that occur in the kidneys, renal pelvis, ureter, urinary bladder, and/or urethra. Commonly, kidney and/or bladder stones contain or consist of calcium salt particles including, but not limited to, calcium oxalate particles and calcium phosphate particles (e.g., apatite particles or brushite particles). Kidney and/or bladder stones can also contain or consist of uric acid, struvite (i.e., NH₄MgPO₄·6H₂O particles), and cystine (i.e., particles containing oxidized cysteine disulfide dimer). Kidney and/or bladder stones typically range in size from less than a millimeter in their largest dimension to 5 or more centimeters in their largest dimension. Kidney stones often form in the kidney or renal pelvis and, when they are small enough (e.g., less than 5 mm), they can pass through the ureter, bladder, and urethra to be eliminated from the body via urination. Kidney and/or bladder stones often cause severe pain in the side and back, below the ribs, and severe pain in the lower abdomen and groin. Other symptoms of kidney and/or bladder stones include, but are not limited to, pain upon urination, abnormally colored urine (e.g., pink, red, or brown), cloudy urine, foul-smelling urine, nausea and vomiting, a persistent need to urinate, low urine volume, fever, and chills. The presence of kidney and/or bladder stones in the urinary system can be confirmed using imaging techniques such as abdominal X-ray, CT scan, and ultrasound.

“Inhibition”, “inhibits”, and “inhibitor” refer to a compound that prohibits or a method of prohibiting, a specific action or function. Inhibition may be partial or complete inhibition. Inhibition may be prophylactic or preventative.

“Administering” refers to therapeutic provision of the compound or a form thereof to a subject, such as by oral administration.

“Treat”, “treatment”, and “treating” refer to an approach for obtaining beneficial or desired results including but not limited to a therapeutic benefit. By therapeutic benefit is meant eradication, elimination, improvement, or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. Treatment includes causing the clinical symptoms of the disease to slow in development by administration of a composition; suppressing the disease, that is, causing a reduction in the clinical symptoms of the disease; inhibiting the disease, that is, arresting the development of clinical symptoms by administration of a composition after the initial appearance of symptoms; and/or relieving the disease, that is, causing the regression of clinical symptoms by administration of a composition after their initial appearance. For example, certain methods described herein treat kidney stones by decreasing or reducing the occurrence, frequency, or progression of kidney stone formation; or treat kidney stones by decreasing the size of a kidney stone.

An “effective amount,” “therapeutically effective amount,” or a “pharmaceutically effective amount” is an amount sufficient to accomplish a stated purpose (e.g., achieve the effect for which it is administered, treat a disease, reduce enzyme activity, reduce one or more symptoms of a disease or condition). An example of an “effective amount” is an amount sufficient to contribute to the treatment, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). Efficacy can also be expressed as “-fold” increase or decrease. For example, a therapeutically effective amount can have at least a 1.2-fold, 1.5-fold, 2-fold, 5-fold, or more effect over a control. In some examples, an effective amount may be an amount effective to prevent kidney stone development or progression, reduce or decrease kidney stone occurrence or frequency, treat kidney stone disease, treat PH1, or reduce, ameliorate, delay the progression of, delay the onset of, eliminate, or eradicate one or more symptoms of PH1.

“Patient” or “subject” or “subject in need thereof” refers to a living organism suffering from or prone to a disease or condition that can be treated or improved by using the methods provided herein. The term does not necessarily indicate that the subject has been diagnosed with a particular disease, but typically refers to an individual under medical supervision. Non-limiting examples include humans, other mammals, rats, mice, dogs, non-human primates, monkeys, goat, sheep, cows, deer, and other non-mammalian animals. In some embodiments, a patient, subject, or subject in need thereof is human. In some embodiments, human is an adult (e.g., at least 18 years of age). In some embodiments, human is an adolescent of at least 12 years old with a body weight of at least 50 kg. In some embodiments, human is a child from 6 to 12 years old or having a body weight of less than 50 kg.

Unless specifically indicated otherwise, the content of Compound lain, e.g., a tablet formulation, is calculated based on the weight of Compound 1a, including the salt (e.g., sodium) and water content (e.g., monohydrate).

### III. Crystalline Forms

In one aspect, the present disclosure provides 1) crystalline Form A of Compound 1a or a tautomer thereof; 2) crystalline Form D or E of Compound 1b, or a tautomer and/or hydrate thereof; and 3) co-crystalline Form C of Compound 1 and Compound 1b, or a tautomer thereof, each of which is represented by the formula:

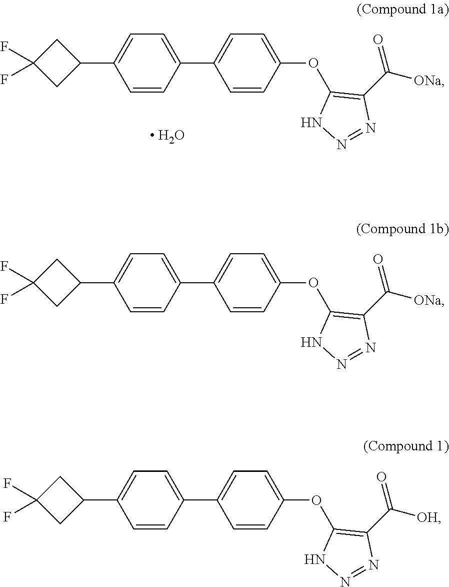

or a tautomer thereof, wherein each of crystalline Forms A, D, E, and C is characterized by an X-ray powder diffraction (XRPD) pattern as described herein.

Methods for collection of XRPD data are known in the art, and any such methods can be used for characterizing the crystalline forms of Compounds 1, 1a, and/or 1b. For example, the X-ray powder diffraction patterns described herein can be generated using Cu Kα1 radiation.

In some embodiments, the crystalline form described herein is further characterized by a differential scanning calorimetry (DSC) thermogram. In some embodiments, a DSC thermogram is recorded using a sample weight of a few milligrams (e.g., about 1-2 mg), which is subjected to temperatures ranging from room temperature to 350° C. using a ramp of 10° C. per minute under nitrogen.

In some embodiments, the crystalline form described herein is further characterized by a thermal gravimetric analysis (TGA). In some embodiments, a TGA thermogram is recorded using a sample weight of a few milligrams (e.g., about 2-10 mg), which is subjected to temperatures ranging from room temperature to 350° C. using a ramp of 10° C. per minute under nitrogen.

In some embodiments, the crystalline form described herein is further characterized by a dynamic vapor sorption (DVS). In some embodiments, the DVS is recorded using a sample weight of a few milligrams (e.g., about 20 mg) on an ADVENTURE series DVS at 25° C. under nitrogen. In some embodiments, the sample is subjected to the cycle as follows: 1) ambient relative humidity (RH) to 90% RH at a rate of 10% RH and from 90% RH to 95% RH in a rate of 5% RH; 2) 95% RH to 90% RH at a rate of 5% RH and 90% RH to 0% RH at a rate of 10% RH; and 3) 0% RH to 90% RH at a rate of 10% RH and 90% RH to 95% RH in a rate of 5% RH.

In some embodiments, the crystalline form described herein is further characterized by a polarized light microscopy (PLM). In some embodiments, the PLM is recorded on an Olympus BX53M polarized light microscope at room temperature.

In some embodiments, the crystalline form described herein is further characterized by an inductively coupled plasma-optical emission spectroscopy (ICP-OES) for the content of a sodium salt. In some embodiments, the ICP-OES is recorded using a sample of about 100 mg on an ICP-OES Avio 200 by a method as described in Section VIII. EXAMPLES—“Instruments and Methods”.

### III-1. Crystalline Form A of Compound 1a

In one embodiment, the present disclosure provides crystalline Form A of Compound 1a represented by the formula:

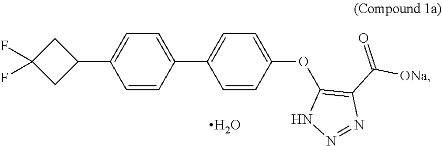

or a tautomer thereof, characterized by an X-ray powder diffraction (XRPD) pattern including peaks at 6.2, 9.2, 15.4, 28.0, and 29.3 degrees 2θ (each ±0.2 degrees 2θ). In some embodiments, the X-ray powder diffraction (XRPD) pattern further includes three or more peaks at 22.8, 24.9, 25.8, 27.3, 31.2, 31.7, and 34.4 degrees 2θ (each ±0.2 degrees 2θ). In some embodiments, crystalline Form A is characterized by an X-ray powder diffraction (XRPD) pattern including peaks (i.e., the first 10 peaks ranked according to relative peak intensity %) at 6.2, 9.2, 15.4, 22.8, 24.9, 25.8, 27.3, 28.0, 29.3, and 34.4 (each ±0.2 degrees 2θ). In some embodiments, crystalline Form A is characterized by an X-ray powder diffraction (XRPD) pattern including three, four, five or more peaks listed in Table 5. In some embodiments, crystalline Form A is characterized by an X-ray powder diffraction (XRPD) pattern including at least five peaks listed in Table 5. In some embodiments, crystalline Form A is characterized by an X-ray powder diffraction (XRPD) pattern including at least ten peaks listed in Table 5.

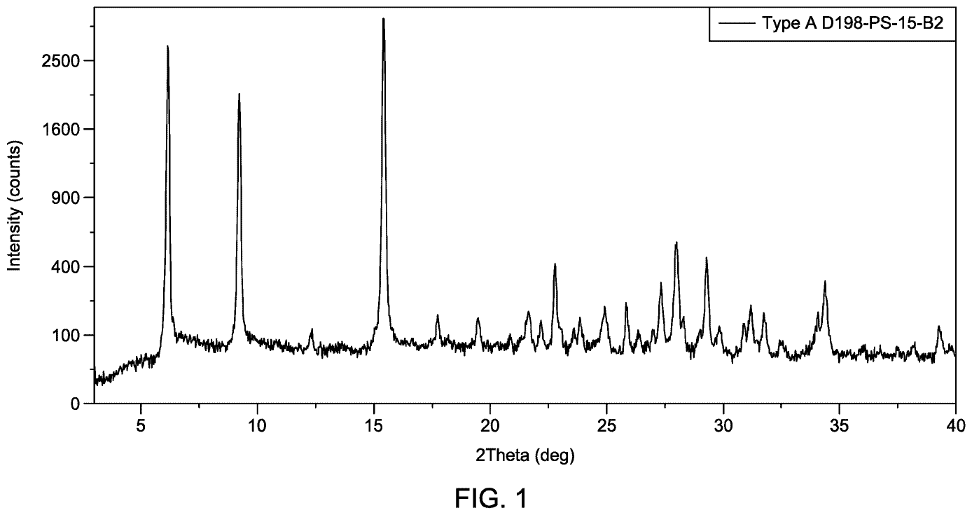

In some embodiments, crystalline Form A is characterized by an X-ray powder diffraction pattern substantially in accordance with FIG. 1.

In some embodiments, crystalline Form A is substantially free of one or more crystalline or amorphous forms of Compound 1 represented by the formula:

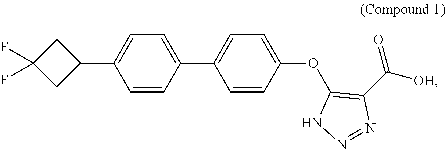 [SMILES: O=C(O)c1nn[nH]c1Oc1ccc(-c2ccc(C3CC(F)(F)C3)cc2)cc1]

a tautomer, a sodium salt, a hydrate, or a combination thereof, provided that the one or more crystalline or amorphous forms of Compound 1 are other than crystalline Form A of Compound 1a. In some embodiments, crystalline Form A is substantially free of other crystalline or amorphous forms of Compound 1, 1a, and/or 1b. In some embodiments, crystalline Form A is substantially free of other crystalline or amorphous forms of Compound 1a. In some embodiments, crystalline Form A is substantially free of other crystalline or amorphous forms of Compound 1, 1a, and 1b.

In some embodiments, crystalline Form A is further characterized by a differential scanning calorimetry (DSC) thermogram including one or more endothermic peaks at about 118.5° C., about 240.8° C., and/or about 243.4° C. In some embodiments, crystalline Form A is further characterized by a differential scanning calorimetry (DSC) thermogram including endothermic peaks at about 118.5° C. and about 240.8° C. In some embodiments, crystalline Form A is further characterized by a differential scanning calorimetry (DSC) thermogram including endothermic peaks at about 118.5° C., about 240.8° C., and about 243.4° C. In some embodiments, the differential scanning calorimetry (DSC) thermogram includes an endothermic peak at about 118.5° C. In some embodiments, the endothermic peak at about 118.5° C. has an onset temperature of about 89.6° C. In some embodiments, the differential scanning calorimetry (DSC) thermogram comprises an endothermic peak at about 240.81° C. In some embodiments, the endothermic peak at about 240.81° C. has an onset temperature of about 238.1° C. In some embodiments, the differential scanning calorimetry (DSC) thermogram includes an endothermic peak at about 243.4° C.

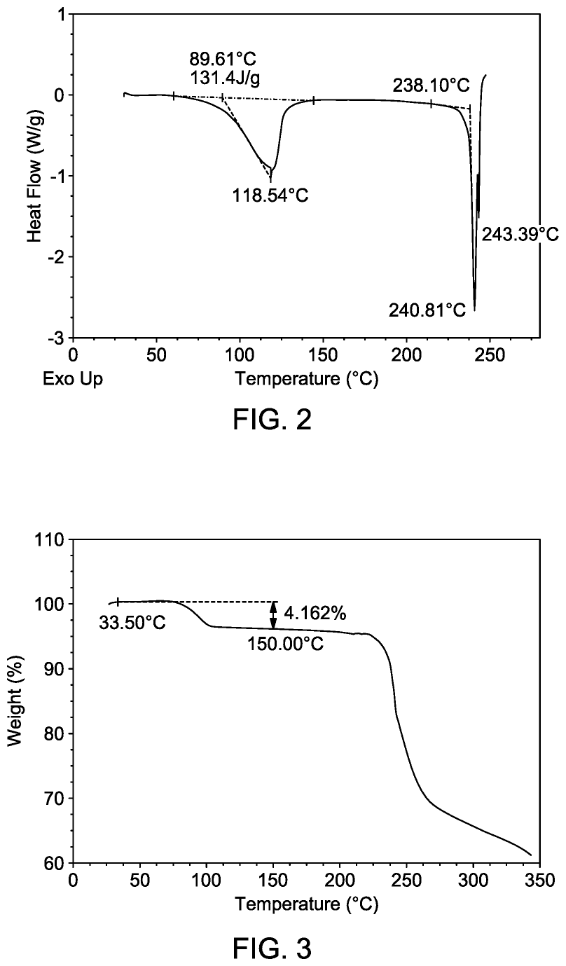

In some embodiments, crystalline Form A is further characterized by a differential scanning calorimetry (DSC) thermogram substantially in accordance with FIG. 2.

In some embodiments, crystalline Form A is further characterized by a weight loss of from about 3% to about 5% upon heating to about 150° C., as measured by a thermal gravimetric analysis (TGA). In some embodiments, crystalline Form A is further characterized by a weight loss of about 4.2% upon heating from about 33.5° C. to about 150.0° C., as measured by the thermal gravimetric analysis (TGA).

In some embodiments, crystalline Form A is further characterized by a thermal gravimetric analysis (TGA) thermogram substantially in accordance with FIG. 3.

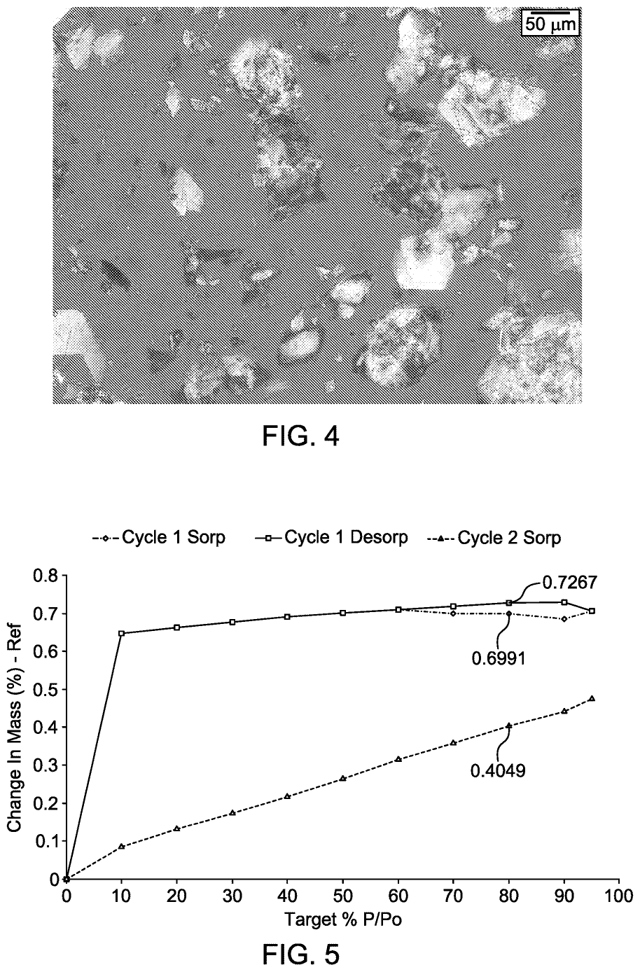

In some embodiments, crystalline Form A is further characterized by a polarized light microscopy (PLM) image substantially in accordance with FIG. 4.

In some embodiments, crystalline Form A is further characterized by: i) a weight gain of less than about 0.1% after undergoing a first dynamic vapor sorption cycle from about 10% relative humidity (RH) to about 95% RH at 25° C.; and/or ii) a weight loss of less than about 0.1% after undergoing a first dynamic vapor desorption cycle from about 95% relative humidity (RH) to about 10% RH at 25° C. In some embodiments, crystalline Form A is further characterized by: i) a weight gain of less than about 0.03% after undergoing the first dynamic vapor desorption cycle from about 60% relative humidity (RH) to about 80% RH at 25° C.; and/or ii) a weight loss of less than about 0.03% after undergoing the first dynamic vapor desorption cycle from about 80% relative humidity (RH) to about 60% RH at 25° C.

In some embodiments, crystalline Form A is further characterized by a dynamic vapor sorption profile substantially as shown in FIG. 5.

In some embodiments, Compound 1a of crystalline Form A is in a monohydrate form. In some embodiments, Compound 1a of crystalline Form A is in a monohydrate form, characterized by a weight loss of about 4.2% upon heating from about 33.5° C. to about 150.0° C., as measured by the thermal gravimetric analysis (TGA).

In some embodiments, Compound 1a of crystalline Form A is in a mono-sodium salt form, characterized by a sodium content of about 5.4% by weight, as measured by an inductively coupled plasma-optical emission spectroscopy (ICP-OES).

In some embodiments, crystalline Form A is non-hygroscopic. In some embodiments, crystalline Form A is non-hygroscopic, characterized by a dynamic vapor sorption, wherein i) a weight gain of less than about 0.1% after undergoing a first dynamic vapor sorption cycle from about 10% relative humidity (RH) to about 95% RH at 25° C.; and/or ii) a weight loss of less than about 0.1% after undergoing a first dynamic vapor desorption cycle from about 95% relative humidity (RH) to about 10% RH at 25° C.

In some embodiments, Compound 1a of crystalline Form A is in a monohydrate form and non-hygroscopic. In some embodiments, Compound 1a of crystalline Form A is in a mono-sodium salt, monohydrate form. In some embodiments, Compound 1a of crystalline Form A is in a mono-sodium salt, monohydrate form and non-hygroscopic.

In some embodiments, crystalline Form A is characterized by an X-ray powder diffraction pattern substantially in accordance with FIG. 1; and is further characterized by a differential scanning calorimetry (DSC) thermogram substantially in accordance with FIG. 2. In some embodiments, crystalline Form A is characterized by an X-ray powder diffraction pattern substantially in accordance with FIG. 1; is further characterized by a differential scanning calorimetry (DSC) thermogram substantially in accordance with FIG. 2; and is further characterized by a thermal gravimetric analysis (TGA) thermogram substantially in accordance with FIG. 3. In some embodiments, crystalline Form A is characterized by an X-ray powder diffraction pattern substantially in accordance with FIG. 1; is further characterized by a differential scanning calorimetry (DSC) thermogram substantially in accordance with FIG. 2; is further characterized by a thermal gravimetric analysis (TGA) thermogram substantially in accordance with FIG. 3; and is further characterized by a polarized light microscopy (PLM) image substantially in accordance with FIG. 4. In some embodiments, crystalline Form A is characterized by an X-ray powder diffraction pattern substantially in accordance with FIG. 1; is further characterized by a differential scanning calorimetry (DSC) thermogram substantially in accordance with FIG. 2; is further characterized by a thermal gravimetric analysis (TGA) thermogram substantially in accordance with FIG. 3; is further characterized by a polarized light microscopy (PLM) image substantially in accordance with FIG. 4; and is further characterized by a dynamic vapor sorption profile substantially as shown in FIG. 5.

### III-2. Crystalline Form D of Compound 1b

In one embodiment, the present disclosure provides crystalline Form D of Compound 1b represented by the formula:

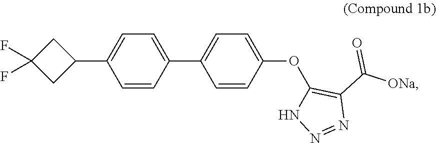

or a tautomer thereof, characterized by an X-ray powder diffraction (XRPD) pattern including peaks at 5.0, 10.0, 12.5, 17.6, and 22.7 degrees 2θ (each ±0.2 degrees 2θ). In some embodiments, the X-ray powder diffraction (XRPD) pattern further includes peaks at 7.5, 15.1, 20.1, 22.3, and 25.3 degrees 2θ (each ±0.2 degrees 2θ). In some embodiments, crystalline Form D is characterized by an X-ray powder diffraction (XRPD) pattern including peaks (i.e., the first 10 peaks ranked according to relative peak intensity %) at 5.0, 7.5, 10.0, 12.5, 15.1, 17.6, 20.1, 22.3, 22.7, and 25.3 degrees 2θ (each ±0.2 degrees 2θ). In some embodiments, crystalline Form D is characterized by an X-ray powder diffraction (XRPD) pattern including three, four, five or more peaks listed in Table 9. In some embodiments, crystalline Form D is characterized by an X-ray powder diffraction (XRPD) pattern including at least five peaks listed in Table 9. In some embodiments, crystalline Form D is characterized by an X-ray powder diffraction (XRPD) pattern including at least ten peaks listed in Table 9.

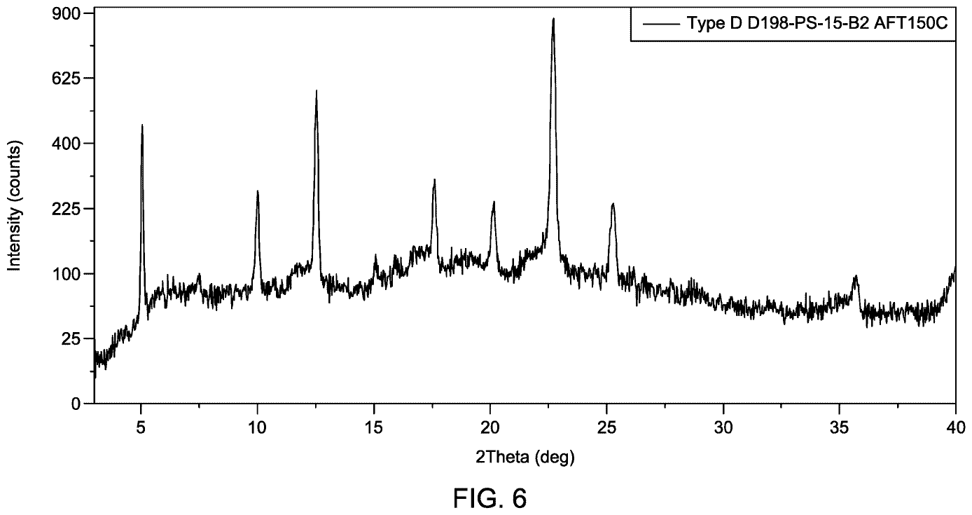

In some embodiments, crystalline Form D is characterized by an X-ray powder diffraction pattern substantially in accordance with FIG. 6.

In some embodiments, crystalline Form D is substantially free of one or more crystalline or amorphous forms of Compound 1 represented by the formula:

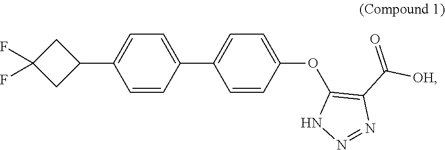 [SMILES: O=C(O)c1nn[nH]c1Oc1ccc(-c2ccc(C3CC(F)(F)C3)cc2)cc1]

a tautomer, a sodium salt, a hydrate, or a combination thereof, provided that the one or more crystalline or amorphous forms of Compound 1 are other than crystalline Form D of Compound 1b. In some embodiments, crystalline Form D is substantially free of other crystalline or amorphous forms of Compound 1, 1a, and/or 1b. In some embodiments, crystalline Form D is substantially free of other crystalline or amorphous forms of Compounds 1, 1a, and 1b.

In some embodiments, crystalline Form D is further characterized by a differential scanning calorimetry (DSC) thermogram including one or more endothermic peaks at about 241.2° C. and/or about 243.2° C. In some embodiments, crystalline Form D is further characterized by a differential scanning calorimetry (DSC) thermogram including endothermic peaks at about 241.2° C. and about 243.2° C. In some embodiments, the differential scanning calorimetry (DSC) thermogram includes an endothermic peak at about 241.2° C. In some embodiments, the endothermic peak at about 241.2° C. has an onset temperature of about 238.4° C.

In some embodiments, the differential scanning calorimetry (DSC) thermogram includes an endothermic peak at about 243.2° C.

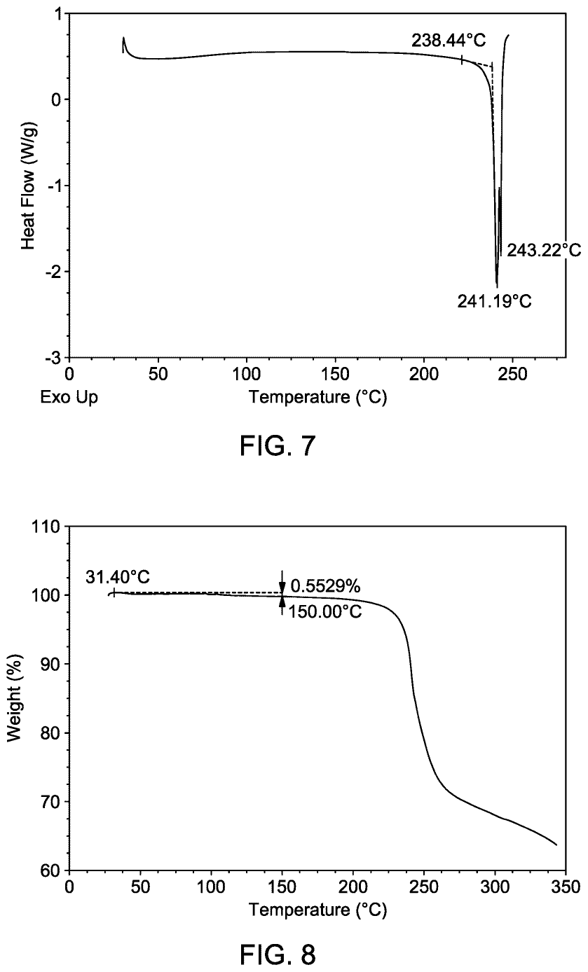

In some embodiments, crystalline Form D is further characterized by a differential scanning calorimetry (DSC) thermogram substantially in accordance with FIG. 7.

In some embodiments, crystalline Form D is further characterized by a weight loss of less than about 1% upon heating to about 150° C., as measured by a thermal gravimetric analysis (TGA). In some embodiments, crystalline Form D is further characterized by a weight loss of about 0.6% upon heating from about 31.4° C. to about 150.0° C., as measured by the thermal gravimetric analysis (TGA).

In some embodiments, crystalline Form D is further characterized by a thermal gravimetric analysis (TGA) thermogram substantially in accordance with FIG. 8.

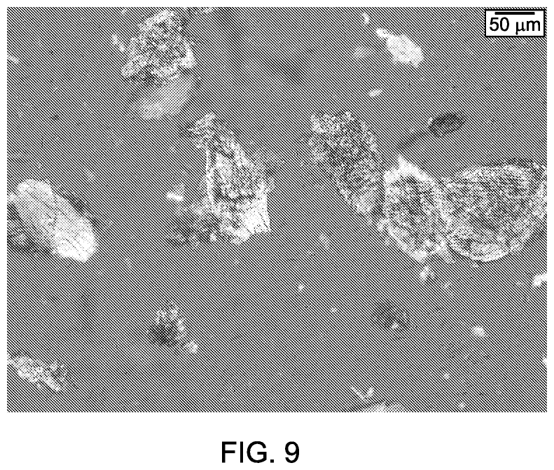

In some embodiments, crystalline Form D is further characterized by a polarized light microscopy (PLM) image substantially in accordance with FIG. 9.

In some embodiments, Compound 1b of crystalline Form D is in an anhydrate form. In some embodiments, Compound 1b of crystalline Form D is in an anhydrate form, characterized by a weight loss of about 0.6% upon heating from about 31.4° C. to about 150.0° C., as measured by the thermal gravimetric analysis (TGA).

In some embodiments, Compound 1b of crystalline Form D is in a mono-sodium salt form. In some embodiments, Compound 1b of crystalline Form D is in a mono-sodium salt, anhydrate form.

In some embodiments, crystalline Form D is characterized by an X-ray powder diffraction pattern substantially in accordance with FIG. 6; and is further characterized by a differential scanning calorimetry (DSC) thermogram substantially in accordance with FIG. 7. In some embodiments, crystalline Form D is characterized by an X-ray powder diffraction pattern substantially in accordance with FIG. 6; is further characterized by a differential scanning calorimetry (DSC) thermogram substantially in accordance with FIG. 7; and is further characterized by a thermal gravimetric analysis (TGA) thermogram substantially in accordance with FIG. 8. In some embodiments, crystalline Form D is characterized by an X-ray powder diffraction pattern substantially in accordance with FIG. 6; is further characterized by a differential scanning calorimetry (DSC) thermogram substantially in accordance with FIG. 7; is further characterized by a thermal gravimetric analysis (TGA) thermogram substantially in accordance with FIG. 8; and is further characterized by a polarized light microscopy (PLM) image substantially in accordance with FIG. 9.

### III-3. Crystalline Form E of Compound 1b

In one embodiment, the present disclosure provides crystalline Form E of Compound 1b represented by the formula:

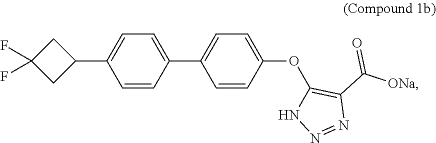

or a tautomer thereof, characterized by an X-ray powder diffraction (XRPD) pattern including three or more peaks at 5.3, 12.7, 16.0, 19.6, 23.0, 25.1, and 28.9 degrees 2θ (each ±0.2 degrees 2θ). In some embodiments, the X-ray powder diffraction (XRPD) pattern includes a peak at 16.0 degrees 2θ (±0.2 degrees 2θ). In some embodiments, crystalline Form E is characterized by an X-ray powder diffraction (XRPD) pattern including a peak at 16.0 degrees 2θ (±0.2 degrees 2θ). In some embodiments, crystalline Form E is characterized by an X-ray powder diffraction (XRPD) pattern including one, two, three, four, five or more peaks listed in Table 11. In some embodiments, crystalline Form E is characterized by an X-ray powder diffraction (XRPD) pattern including at least two peaks listed in Table 11.

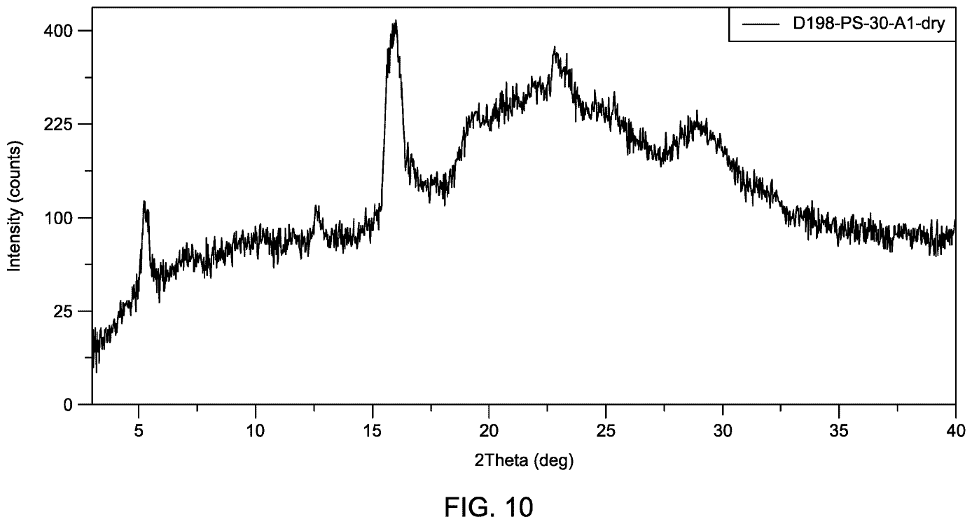

In some embodiments, crystalline Form E is characterized by an X-ray powder diffraction pattern substantially in accordance with FIG. 10.

In some embodiments, crystalline Form E is substantially free of one or more crystalline or amorphous forms of Compound 1 represented by the formula:

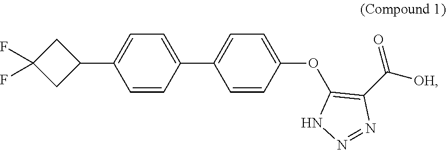 [SMILES: O=C(O)c1nn[nH]c1Oc1ccc(-c2ccc(C3CC(F)(F)C3)cc2)cc1]

a tautomer, a sodium salt, a hydrate, or a combination thereof, provided that the one or more crystalline or amorphous forms of Compound 1 are other than crystalline Form E of Compound 1b. In some embodiments, crystalline Form E is substantially free of other crystalline or amorphous forms of Compound 1, 1a, and/or 1b. In some embodiments, crystalline Form E is substantially free of other crystalline or amorphous forms of Compounds 1, 1a, and 1b.

In some embodiments, crystalline Form E is further characterized by a differential scanning calorimetry (DSC) thermogram including one or more endothermic peaks at about 64.5° C. and/or about 239.3° C. In some embodiments, crystalline Form E is further characterized by a differential scanning calorimetry (DSC) thermogram including endothermic peaks at about 64.5° C. and about 239.3° C. In some embodiments, the differential scanning calorimetry (DSC) thermogram includes an endothermic peak at about 64.5° C. In some embodiments, the differential scanning calorimetry (DSC) thermogram includes an endothermic peak at about 239.3° C. In some embodiments, the endothermic peak at about 239.3° C. has an onset temperature of about 232.5° C.

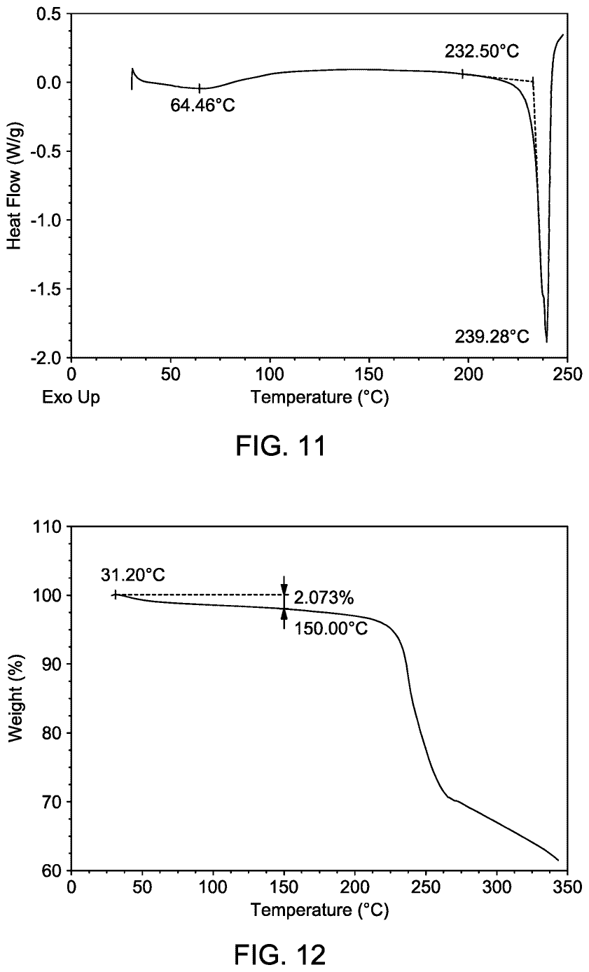

In some embodiments, crystalline Form E is further characterized by a differential scanning calorimetry (DSC) thermogram substantially in accordance with FIG. 11.

In some embodiments, crystalline Form E is further characterized by a weight loss of from about 1.5% to about 3% upon heating to about 150° C., as measured by a thermal gravimetric analysis (TGA). In some embodiments, crystalline Form E is further characterized by a weight loss of about 2.1% upon heating from about 31.2° C. to about 150.0° C., as measured by the thermal gravimetric analysis (TGA).

In some embodiments, crystalline Form E is further characterized by a thermal gravimetric analysis (TGA) thermogram substantially in accordance with FIG. 12.

In some embodiments, Compound 1b of crystalline Form E is in a hemihydrate form. In some embodiments, Compound 1b of crystalline Form E is in an anhydrate form, characterized by a weight loss of about 2.10% upon heating from about 31.2° C. to about 150.0° C., as measured by the thermal gravimetric analysis (TGA).

In some embodiments, Compound 1b of crystalline Form E is in a mono-sodium salt form. In some embodiments, Compound 1b of crystalline Form E is in a mono-sodium salt, hemihydrate form.

In some embodiments, crystalline Form E is characterized by an X-ray powder diffraction pattern substantially in accordance with FIG. 10; and is further characterized by a differential scanning calorimetry (DSC) thermogram substantially in accordance with FIG. 11.

In some embodiments, crystalline Form E is characterized by an X-ray powder diffraction pattern substantially in accordance with FIG. 10; is further characterized by a differential scanning calorimetry (DSC) thermogram substantially in accordance with FIG. 11; and is further characterized by a thermal gravimetric analysis (TGA) thermogram substantially in accordance with FIG. 12.

### III-4. Co-Crystalline Form C of Compounds 1 and 1b

In one embodiment, the present disclosure provides co-crystalline Form C of Compound 1 represented by the formula:

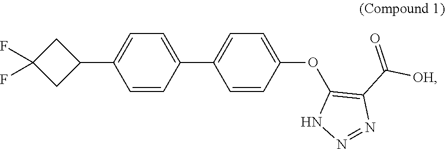 [SMILES: O=C(O)c1nn[nH]c1Oc1ccc(-c2ccc(C3CC(F)(F)C3)cc2)cc1]

and Compound 1b represented by the formula:

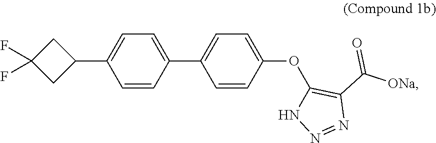

or a tautomer thereof, characterized by an X-ray powder diffraction (XRPD) pattern comprising peaks at 6.3, 16.0, 21.6, 25.9, and 28.8 degrees 2θ (each ±0.2 degrees 2θ). In some embodiments, the X-ray powder diffraction (XRPD) pattern further includes peaks at 18.7, 23.6, 24.4, 26.5, and 31.7 degrees 2θ (each ±0.2 degrees 2θ). In some embodiments, the X-ray powder diffraction (XRPD) pattern further includes peaks at 17.0, 19.8, 20.3, 33.9, and 34.1 degrees 2θ (each ±0.2 degrees 2θ). In some embodiments, co-crystalline Form C is characterized by an X-ray powder diffraction (XRPD) pattern including peaks (i.e., the first 10 peaks ranked according to relative peak intensity %) at 6.3, 16.0, 18.7, 21.6, 23.6, 24.4, 25.9, 26.5, 28.8, and 31.7 degrees 2θ (each ±0.2 degrees 2θ). In some embodiments, co-crystalline Form C is characterized by an X-ray powder diffraction (XRPD) pattern including three, four, five or more peaks listed in Table 13. In some embodiments, co-crystalline Form C is characterized by an X-ray powder diffraction (XRPD) pattern including at least five peaks listed in Table 13. In some embodiments, co-crystalline Form C is characterized by an X-ray powder diffraction (XRPD) pattern including at least ten peaks listed in Table 13.

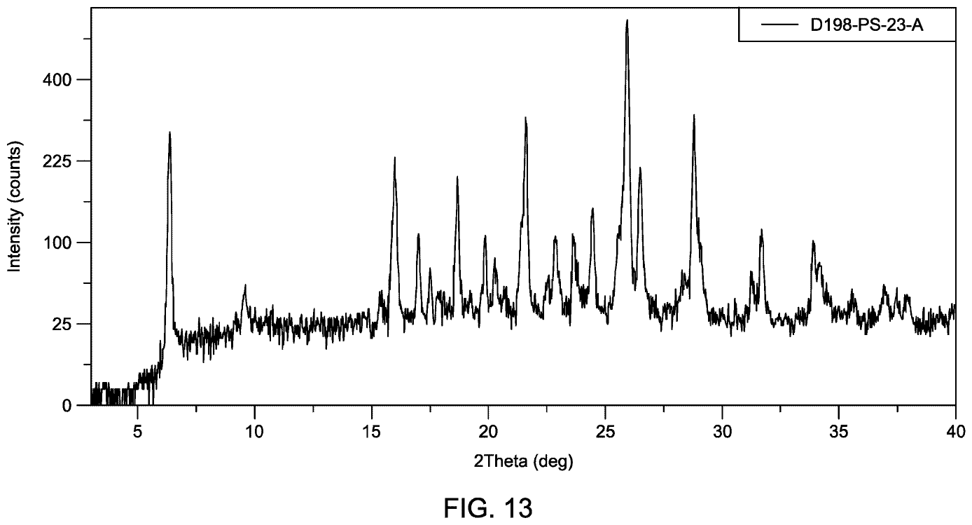

In some embodiments, co-crystalline Form C is characterized by an X-ray powder diffraction pattern substantially in accordance with FIG. 13.

In some embodiments, co-crystalline Form C is substantially free of one or more crystalline or amorphous forms of Compound 1 represented by the formula:

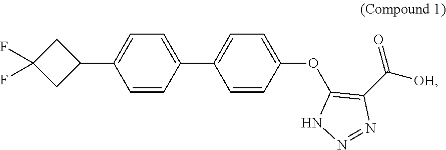 [SMILES: O=C(O)c1nn[nH]c1Oc1ccc(-c2ccc(C3CC(F)(F)C3)cc2)cc1]

a tautomer, a sodium salt, a hydrate, or a combination thereof, provided that the one or more crystalline or amorphous forms of Compound 1 are other than co-crystalline Form C of Compound 1 and Compound 1b. In some embodiments, co-crystalline Form C is substantially free of other crystalline or amorphous forms of Compound 1, 1a, and/or 1b. In some embodiments, co-crystalline Form C is substantially free of other crystalline or amorphous forms of Compounds 1, 1a, and 1b.

In some embodiments, co-crystalline Form C is further characterized by a differential scanning calorimetry (DSC) thermogram including one or more endothermic peaks at about 196.9° C. and/or about 215.1° C. In some embodiments, co-crystalline Form C is further characterized by a differential scanning calorimetry (DSC) thermogram including endothermic peaks at about 196.9° C. and about 215.1° C. In some embodiments, the differential scanning calorimetry (DSC) thermogram includes an endothermic peak at about 196.9° C. In some embodiments, the endothermic peak at about 239.3° C. has an onset temperature of about 193.0° C. In some embodiments, the differential scanning calorimetry (DSC) thermogram includes an endothermic peak at about 215.1° C.

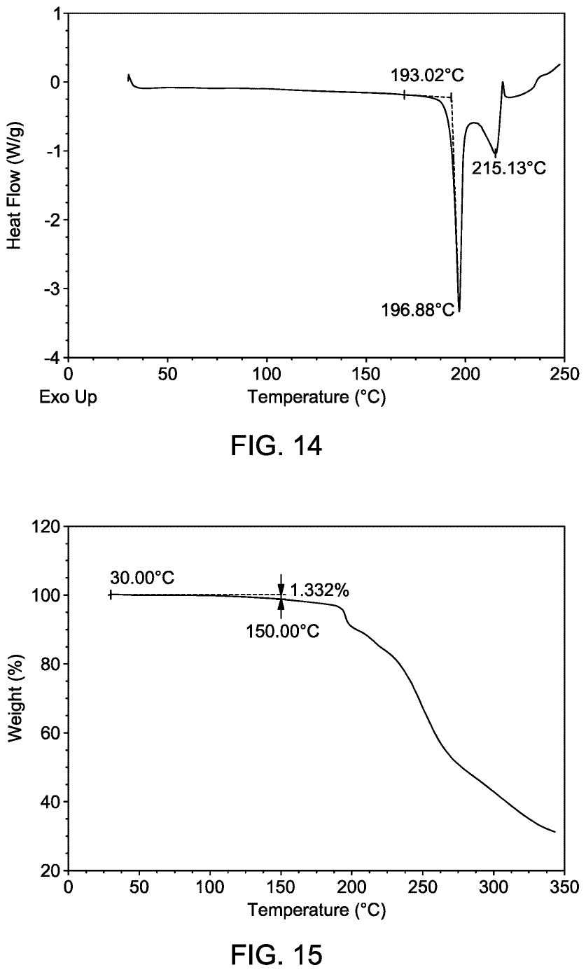

In some embodiments, co-crystalline Form C is further characterized by a differential scanning calorimetry (DSC) thermogram substantially in accordance with FIG. 14.

In some embodiments, co-crystalline Form C is further characterized by a weight loss of from about 1% to about 2% upon heating to about 150° C., as measured by a thermal gravimetric analysis (TGA). In some embodiments, co-crystalline Form C is further characterized by a weight loss of about 1.3% upon heating from about 30.0° C. to about 150.0° C., as measured by the thermal gravimetric analysis (TGA).

In some embodiments, co-crystalline Form C is further characterized by a thermal gravimetric analysis (TGA) thermogram substantially in accordance with FIG. 15.

In some embodiments, Compounds 1 and 1b of co-crystalline Form C are each in an anhydrate form. In some embodiments, Compounds 1 and 1b of co-crystalline Form C are each in an anhydrate form, characterized by a weight loss of about 1.3% upon heating from about 30.0° C. to about 150.0° C., as measured by the thermal gravimetric analysis (TGA).

In some embodiments, Compound 1 of co-crystalline Form C is in an acid form represented by the formula of Compound 1; and Compound 1b of co-crystalline Form C is in a mono-sodium salt form. In some embodiments, Compound 1 of co-crystalline Form C is in an acid, anhydrate form; and Compound 1b of co-crystalline Form C is in a mono-sodium salt, anhydrate form.

In some embodiments, co-crystalline Form C is characterized by an X-ray powder diffraction pattern substantially in accordance with FIG. 13; and is further characterized by a differential scanning calorimetry (DSC) thermogram substantially in accordance with FIG. 14. In some embodiments, co-crystalline Form C is characterized by an X-ray powder diffraction pattern substantially in accordance with FIG. 13; is further characterized by a differential scanning calorimetry (DSC) thermogram substantially in accordance with FIG. 14; and is further characterized by a thermal gravimetric analysis (TGA) thermogram substantially in accordance with FIG. 15.

### III-5. Methods for Preparing Crystalline Form a of Compound 1a

In another aspect, the present disclosure provides methods for preparing crystalline Form A of Compound 1a represented by the formula:

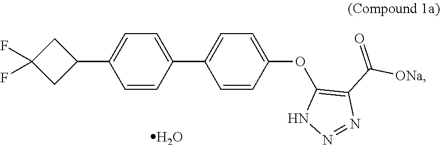

wherein Form A is described according to Section III-1. Crystalline Form A of Compound 1a.

The method can be any one of methods as described in Example 6, for example slurry at room temperature or at 50° C., slow cooling, solid vapor diffusion, anti-solvent addition, and/or polymer-induced crystallization. Starting from Compound 1 represented by the formula:

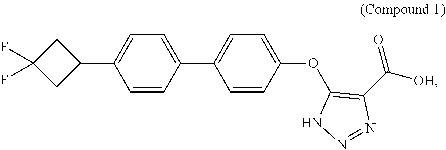 [SMILES: O=C(O)c1nn[nH]c1Oc1ccc(-c2ccc(C3CC(F)(F)C3)cc2)cc1]

a tautomer, a sodium salt, a hydrate, or a combination thereof, in some embodiments, Form A of Compound 1a is prepared by a slurry method as described in Example 6, e.g., including slurrying Compound 1, a tautomer, a sodium salt, a hydrate, or a combination thereof in a solvent (e.g., a solvent system as described in Example 6, C and D) at room temperature or at an elevated temperature (e.g., 50° C.). In some embodiments, Form A of Compound 1a is prepared by a slurry method as described in Example 6, including slurrying Compound 1, a tautomer, a sodium salt, a hydrate, or a combination thereof in a solvent (e.g., a solvent as described in Example 6, C and D) at room temperature. In some embodiments, Form A of Compound 1a is prepared by a slurry method as described in Example 6, including slurrying Compound 1, a tautomer, a sodium salt, a hydrate, or a combination thereof in a solvent (e.g., a solvent as described in Example 6, C and D) at 50° C. In some embodiments, Form A of Compound 1a is prepared by a slow cooling method as described in Example 6, e.g., including cooling a mixture or solution including Compound 1, a tautomer, a sodium salt, a hydrate, or a combination thereof and a solvent (e.g., a solvent system as described in Example 6, C and D) from an elevated temperature (e.g., 50° C.) to a cool temperature (e.g., 5° C.). In some embodiments, Form A of Compound 1a is prepared by a solid vapor diffusion method as described in Example 6, e.g., including exposing Compound 1, a tautomer, a sodium salt, a hydrate, or a combination thereof to a solvent (e.g., a volatile solvent as described in Example 6, D) in a sealed vessel. In some embodiments, Form A of Compound 1a is prepared by an anti-solvent method as described in Example 6, e.g., including 1) dissolving Compound 1, a tautomer, a sodium salt, a hydrate, or a combination thereof in a solvent; and 2) adding an anti-solvent to form a precipitate (e.g., a solvent/anti-solvent system as described in Example 6, D). In some embodiments, Form A of Compound 1a is prepared by a polymer-induced crystallization method as described in Example 6, e.g., including 1) forming a solution including Compound 1, a tautomer, a sodium salt, a hydrate, or a combination thereof in a solvent; 2) adding a polymer (e.g., PVC and/or PVP); and 3) slow evaporating the solvent (e.g., through one or more small holes of a covered vessel including the products of steps 1) and 2)).

In some embodiments, Form A of Compound 1a is prepared according to Example 1.

In some embodiments, the method includes:
a) providing a first mixture including:
   i) Compound 1 represented by the formula:

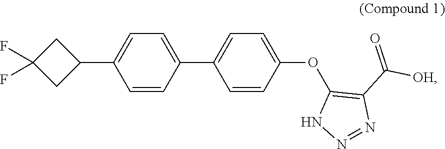 [SMILES: O=C(O)c1nn[nH]c1Oc1ccc(-c2ccc(C3CC(F)(F)C3)cc2)cc1]

a tautomer, a sodium salt, a hydrate, or a combination thereof; and
ii) a solvent mixture including tetrahydrofuran (THF) and water;
b) stirring the first mixture at a temperature of from about 45° C. to about 60° C. to form a second mixture;
c) filtering the second mixture of step b) to provide a third mixture including Compound 1b represented by the formula:

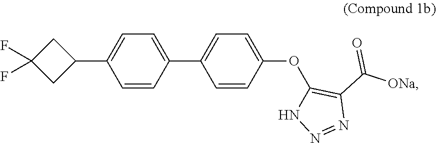

or a tautomer thereof;
d) cooling the third mixture to a temperature of less than 10° C. to form a slurry; and
e) isolating a solid from the slurry to provide the crystalline Form A.

In some embodiments, prior to step d), one or more seeds of crystalline Form A of Compound 1b are added to the third mixture of step c).

In some embodiments, Compound 1, a tautomer, a sodium salt, a hydrate, or a combination thereof is a mixture including Compound 1 (in a hydrate and/or an anhydrate form), a mono-sodium salt (in a hydrate and/or an anhydrate form), a di-sodium salt (in a hydrate, and/or an anhydrate form), and/or a tautomer thereof. In some embodiments, Compound 1, a tautomer, a sodium salt, a hydrate, or a combination thereof is a mixture of Compound 1a (in an amorphous or a crystalline form), Compound 1b (in an amorphous or a crystalline form), and Compound 1 (in an amorphous or a crystalline form), each of which is optionally a tautomer thereof. In some embodiments, Compound 1, a tautomer, a sodium salt, a hydrate, or a combination thereof is a mixture of mono-sodium salt, monohydrate Form A, co-crystalline (an acid and mono-sodium salt) anhydrate Form C, and an acid (represented by the formula of Compound 1).

With reference to step a), in some embodiments, the solvent mixture has a THF:H₂O ratio of from about 1:1 to about 1:5, from about 1:2 to about 1:4, or about 1:3 by volume. In some embodiments, the solvent mixture has a THF:H₂O ratio of about 1:3 by volume.

With reference to step a), in some embodiments, Compound 1, a tautomer, a sodium salt, a hydrate, or a combination thereof is present in the first mixture in a concentration of from about 25 mg/mL to about 50 mg/mL. In some embodiments, Compound 1, a tautomer, a sodium salt, a hydrate, or a combination thereof is present in the first mixture in a concentration of from about 30 mg/mL to about 40 mg/mL. In some embodiments, Compound 1, a tautomer, a sodium salt, a hydrate, or a combination thereof is present in the first mixture in a concentration of about 33 mg/mL. In some embodiments, the mixture of Compound 1a, Compound 1b, and Compound 1 is present in the first mixture in a concentration of from about 25 mg/mL to about 50 mg/mL. In some embodiments, the mixture of Compound 1a, Compound 1b, and Compound 1 is present in the first mixture in a concentration of from about 30 mg/mL to about 40 mg/mL. In some embodiments, the mixture of Compound 1a, Compound 1b, and Compound 1 is present in the first mixture in a concentration of about 33 mg/mL.

In general, step a) is conducted at room temperature; and/or step b) is conducted at an elevated temperature (e.g., from about 45° C. to about 60° C.). In some embodiments, step a) is conducted at room temperature; and/or step b) is conducted at a temperature of about 50° C. In some embodiments, step b) is conducted for a period of from about 1 to about 5 hours. In some embodiments, step b) is conducted at a temperature of about 50° C. for a period of about 3 hours.

With reference to step c), in some embodiments, the third mixture is a solution including Compound 1b or a tautomer thereof.

In some embodiments, step d) is conducted at a temperature of about 5° C. In some embodiments, the slurry is cooled at the temperature of about 5° C. for a period of from about 2 to 3 days. In some embodiments, step d) is conducted at a temperature of about 5° C.; and the slurry is cooled at the temperature of about 5° C. for a period of from about 2 to 3 days.

In general, step e) of isolating a solid can be conduct by a standard isolation method (e.g., filtration, centrifuging, decanting). In some embodiments, step e) is conducted by filtration. In some embodiments, the solid is further dried under a vacuum at room temperature to provide the crystalline Form A of Compound 1a.

In some embodiments, the present disclosure provides a method for preparing crystalline Form A of Compound 1a, as described according to Section III-1. Crystalline Form A of Compound 1a; and the method includes:
a) at room temperature, providing a first mixture comprising i) a mixture of Compound 1a, Compound 1b, and Compound 1, each of which is represented by the formula:

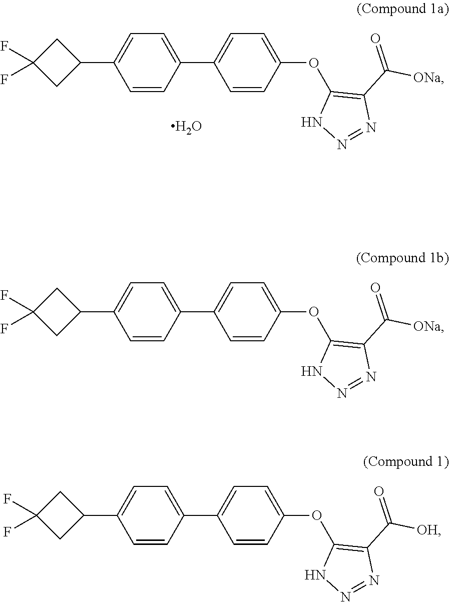

or a tautomer thereof; and ii) a solvent mixture comprising tetrahydrofuran (THF) and water in a THF:H₂O ratio of about 1:3 by volume;
b) stirring the first mixture at a temperature of about 50° C. to form a second mixture;
c) filtering the second mixture of step b) to provide a third mixture comprising Compound 1b or a tautomer thereof; and one or more seeds of crystalline Form A of Compound 1b are added to the third mixture;
d) cooling the third mixture to a temperature of about 5° C. to form a slurry; and
e) isolating a solid from the slurry to provide the crystalline Form A.

In some embodiments, in step a), the mixture of Compound 1a, Compound 1b, and Compound 1 is present in the first mixture in a concentration of about 33 mg/mL.

In some embodiments, step b) is conducted for a period of about 3 hours.

In some embodiments, in step c), the third mixture is a solution.

In some embodiments, in step d), the slurry is cooled at the temperature of about 5° C. for a period of about 2.5 days.

In some embodiments, step e) is conducted by filtration and the solid is further dried under a vacuum at room temperature to provide the crystalline Form A.

In some embodiments, in step a), the mixture of Compound 1a, Compound 1b, and Compound 1 is present in the first mixture in a concentration of about 33 mg/mL; step b) is conducted for a period of about 3 hours; in step c), the third mixture is a solution; in step d), the slurry is cooled at the temperature of about 5° C. for a period of about 2.5 days; and step e) is conducted by filtration and the solid is further dried under a vacuum at room temperature to provide the crystalline Form A.

### IV. Compositions

The crystalline forms as described herein, 1) crystalline Form A of Compound 1a or a tautomer thereof; 2) crystalline Form D or E of Compound 1b, or a tautomer and/or hydrate thereof; and 3) co-crystalline Form C of Compound 1 and Compound 1b, or a tautomer thereof, can be prepared in various compositions suitable for delivery to a subject. A composition suitable for administration to a subject typically includes one or more of crystalline Forms A, D, E, and C, as described herein; and a pharmaceutically acceptable excipient. In some embodiments, the compositions and pharmaceutical compositions provided herein include crystalline Form A of Compound 1a (or a tautomer thereof), which is substantially free of other crystalline or amorphous forms of Compounds 1, 1a, and/or 1b.

In some embodiments, the present disclosure provides a pharmaceutical composition including crystalline Form A of Compound 1a or a tautomer thereof and a pharmaceutically acceptable excipient, wherein Form A is described according to Section III-1. Crystalline Form A of Compound 1a.

The pharmaceutical compositions can be in the form of a sterile injectable aqueous or oleaginous solutions and suspensions. Sterile injectable preparations can be formulated using non-toxic parenterally-acceptable vehicles including water, Ringer's solution, and isotonic sodium chloride solution, and acceptable solvents such as 1,3-butane diol. In addition, sterile, fixed oils are conventionally employed as a solvent or suspending medium. For this purpose, a bland fixed oil such as a synthetic mono- or diglyceride may be used. In addition, fatty acids such as oleic acid may be useful in the preparation of injectables.

Aqueous suspensions contain the active ingredient in admixture with excipients suitable for the manufacture of aqueous suspensions. Such excipients include, but are not limited to, suspending agents such as sodium carboxymethylcellulose, methylcellulose, oleagino-propylmethylcellulose, sodium alginate, polyvinyl-pyrrolidone, gum tragacanth and gum acacia; dispersing or wetting agents such as lecithin, polyoxyethylene stearate, and polyethylene sorbitan monooleate; and preservatives such as ethyl, n-propyl, and p-hydroxybenzoate.

Oily suspensions can be formulated by suspending the active ingredient in a vegetable oil such as, for example, arachis oil, olive oil, sesame oil or coconut oil, or in a mineral oil such as liquid paraffin. The oily suspensions can contain a thickening agent, such as beeswax, hard paraffin, or cetyl alcohol. These compositions can be preserved by the addition of an antioxidant such as ascorbic acid.

Dispersible powders and granules (suitable for preparation of an aqueous suspension by the addition of water) can contain the active ingredient in admixture with a dispersing agent, wetting agent, suspending agent, or combinations thereof. Additional excipients can also be present.

The pharmaceutical compositions including one or more of crystalline Forms A, D, E, and C, as described herein can also be in the form of oil-in-water emulsions. The oily phase can be a vegetable oil such as, for example olive oil or arachis oil, or a mineral oil such as, for example, liquid paraffin, or mixtures of these. Suitable emulsifying agents include, for example, naturally-occurring gums, such as gum acacia and gum tragacanth; naturally-occurring phospholipids such as soy lecithin; esters or partial esters derived from fatty acids and hexitol anhydrides, such as sorbitan monooleate; and condensation products of said partial esters with ethylene oxide, such as polyoxyethylene sorbitan monooleate.

Pharmaceutical compositions containing including one or more of crystalline Forms A, D, E, and C, as described herein can also be in a form suitable for oral use. Suitable compositions for oral administration include, but are not limited to, tablets, troches, lozenges, aqueous or oily suspensions, dispersible powders or granules, emulsions, hard or soft capsules, syrups, elixirs, solutions, buccal patches, oral gels, chewing gums, chewable tablets, effervescent powders, and effervescent tablets. Compositions for oral administration can be formulated according to any method known to those of skill in the art. Such compositions can contain one or more agents selected from sweetening agents, flavoring agents, coloring agents, antioxidants, and preserving agents in order to provide pharmaceutically elegant and palatable preparations.

Tablets generally contain the active ingredient in admixture with non-toxic pharmaceutically acceptable excipients, including, for example, inert diluents, such as cellulose, silicon dioxide, aluminum oxide, calcium carbonate, sodium carbonate, glucose, mannitol, sorbitol, lactose, calcium phosphate, and sodium phosphate; granulating and disintegrating agents, such as corn starch and alginic acid; binding agents, such as polyvinylpyrrolidone (PVP), cellulose, polyethylene glycol (PEG), starch, gelatin, and acacia; and lubricating agents such as magnesium stearate, stearic acid, and talc. The tablets can be uncoated or coated, enterically or otherwise, by known techniques to delay disintegration and absorption in the gastrointestinal tract and thereby provide a sustained action over a longer period. For example, a time delay material such as glyceryl monostearate or glyceryl distearate can be employed. Tablets can also be coated with a semi-permeable membrane and optional polymeric osmogents according to known techniques to form osmotic pump compositions for controlled release.

Compositions for oral administration can be formulated as hard gelatin capsules wherein the active ingredient is mixed with an inert solid diluent (such as calcium carbonate, calcium phosphate, or kaolin), or as soft gelatin capsules wherein the active ingredient is mixed with water or an oil medium (such as peanut oil, liquid paraffin, or olive oil).

In some embodiments, the pharmaceutical composition includes one or more of crystalline Forms A, D, E, and C, as described herein and one or more additional active agents for treating diseases as described herein (e.g., hyperoxaluria, primary hyperoxaluria, type I (PH1), kidney and/or bladder stones). In some embodiments, the pharmaceutical composition includes one or more of crystalline Forms A, D, E, and C, as described herein and one or more additional active agents for preventing the development, worsening, or recurrence of kidney stones. Examples of such active agents include, but are not limited to, thiazides (e.g., bendroflumethiazide, chlorothiazide, chlorthalidone, hydrochlorothiazide, indapamide, methyclothiazide, metolazone, polythiazide, and the like); citrate salts (e.g., sodium citrate, potassium citrate, and the like); phosphate salts (e.g., monopotassium phosphate, dipotassium phosphate, and the like); vitamin B6 compounds (e.g., pyridoxine, pyridoxal, pyridoxamine, and the like); cystine-binding thiol compounds (e.g., α-mercaptopropionylglycine, D-penicillamine, captopril, and the like); purine analog xanthine oxidase inhibitors (e.g., allopurinol, oxypurinol, and the like); and other xanthine oxidase inhibitors (e.g., febuxostate, topiroxostat, and the like).

### V. Methods

The present disclosure provides a method for treating primary hyperoxaluria (PH1), including administering to a subject in need thereof a therapeutically effective amount of Compound 1 or a tautomer thereof including one or more of crystalline Forms A, D, E, and C, as described herein, or a pharmaceutical composition as described herein.

PH1 has a prevalence of 1-3 per million individuals and an incidence of 1-9:100,000 live births per year in Europe [Salido, supra]. PH1 is caused by mutations of the gene encoding peroxisomal enzyme AGT, which fails to detoxify glyoxylate and leads to a marked increase in oxalate synthesis by the liver. In PH1, excreted urinary oxalate (UOx) is elevated leading to the production of insoluble calcium oxalate (CaOx) crystals which tend to precipitate primarily in the kidney, forming kidney stones and diffuse nephrocalcinosis [Kaufman, supra]. This impairs renal function which progresses to end-stage renal disease (ESRD). Once renal function declines to a glomerular filtration rate (GFR) below 30 ml/min/1.73 m², the amount of oxalate produced by the liver can no longer be cleared by the kidneys, leading to systemic deposition of CaOx (oxalosis). In people with PH1, the accumulated oxalate is deposited in the kidneys and urinary tract. It combines with calcium, forming the main component of kidney and bladder stones. First symptoms of PH1 include hematuria, abdominal pain, passage of a stone, or repeated urinary tract infections. The initial diagnosis is based on clinical and sonographic findings, and UOx assessment. AGT activity assessment in a liver biopsy and/or DNA analysis is required to confirm a PH1 diagnosis and to initiate conservative treatment (high fluid intake, pyridoxine, CaOx crystallization inhibitors), aimed at maintaining renal function. The most effective treatment for PH1 is liver transplantation (LTx), alone (pre-emptive) or combined with kidney transplantation [Cochat, et al. (2012) Nephrol Dial Transplant. 27: 1729].

In some embodiments, the method for treating PH1 includes administering Compound 1a or a tautomer thereof including crystalline Form A, and a pharmaceutically acceptable salt thereof; or a pharmaceutical composition including Form A to a subject in need thereof. In some embodiments, the subject has kidney and/or bladder stones. In some embodiments, Compound 1a or a tauomer thereof reduces glyoxylate level in the subject. In some embodiments, Compound 1a or a tautomer thereof reduces an accumulation of oxalate in kidney and/or urinary tract.

Methods for treating kidney and/or bladder stones are also provided herein. The methods include administering to a subject in need thereof a therapeutically effective amount of Compound 1 or a tautomer thereof including one or more of crystalline Forms A, D, E, and C, as described herein, or a pharmaceutical composition as described herein. In some embodiments, the method for treating kidney and/or bladder stones includes administering Compound 1a or a tautomer thereof including crystalline Form A, and a pharmaceutically acceptable salt thereof; or a pharmaceutical composition including Form A to a subject in need thereof. In some embodiments, Compound 1a or a tautomer thereof reduces an accumulation of oxalate in kidney and/or urinary tract.

In one aspect, the present disclosure provides a method of treating hyperoxaluria in a subject in need thereof, the method including administering to the subject a therapeutically effective amount of Compound 1, having the formula:

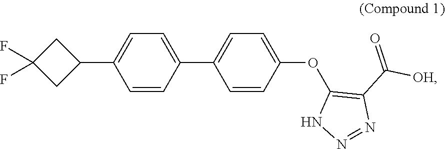 [SMILES: O=C(O)c1nn[nH]c1Oc1ccc(-c2ccc(C3CC(F)(F)C3)cc2)cc1]

a tautomer, a pharmaceutically acceptable salt, a hydrate, or a combination thereof, including one or more of crystalline Forms A, D, E, and C, as described herein, or a pharmaceutical composition including one or more of crystalline Forms A, D, E, and C, as described herein, wherein the therapeutically effective amount reduces a maximal urinary oxalate (uOxalate) excretion: i) to less than about 0.46 millimoles per 1.73 square meters per 24 hours (mmol/1.73 m²/24 hr), or ii) by at least about 50% relative to a uOxalate excretion prior to the treating.

In some embodiments, provided herein is a method of treating hyperoxaluria in a subject in need thereof, the method including administering to the subject a therapeutically effective amount of Compound 1a or a tautomer thereof including crystalline Form A as described herein or a pharmaceutical composition including Form A as described herein, wherein the therapeutically effective amount reduces a maximal urinary oxalate (uOxalate) excretion: i) to less than about 0.46 millimoles per 1.73 square meters per 24 hours (mmol/1.73 m²/24 hr)), or ii) by at least about 50% relative to a uOxalate excretion prior to the treating.

In one aspect, the present disclosure provides a method of treating hyperoxaluria in a subject in need thereof, the method including administering to the subject a therapeutically effective amount of Compound 1, having the formula:

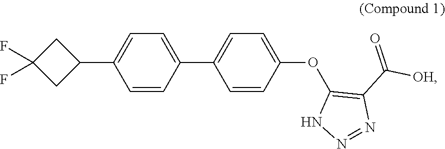 [SMILES: O=C(O)c1nn[nH]c1Oc1ccc(-c2ccc(C3CC(F)(F)C3)cc2)cc1]

a tautomer, a pharmaceutically acceptable salt, a hydrate, or a combination thereof, including one or more of crystalline Forms A, D, E, and C, as described herein, or a pharmaceutical composition including one or more of crystalline Forms A, D, E, and C, as described herein, wherein the therapeutically effective amount is a total daily dosage of about 20 milligrams (mg) to about 2,500 mg.

In some embodiments, provided herein is a method of treating hyperoxaluria in a subject in need thereof, the method including administering to the subject a therapeutically effective amount of Compound 1a or a tautomer thereof including crystalline Form A as described herein or a pharmaceutical composition including Form A as described herein, wherein the therapeutically effective amount is a total daily dosage of about 20 mg to 2,500 mg.

In another aspect, the present disclosure provides a method of treating kidney stones in a subject in need thereof, the method including administering to the subject a therapeutically effective amount of Compound 1, having the formula:

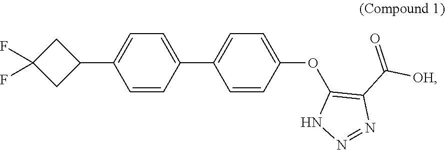 [SMILES: O=C(O)c1nn[nH]c1Oc1ccc(-c2ccc(C3CC(F)(F)C3)cc2)cc1]

a tautomer, a pharmaceutically acceptable salt, a hydrate, or a combination thereof, including one or more of crystalline Forms A, D, E, and C, as described herein, or a pharmaceutical composition including one or more of crystalline Forms A, D, E, and C, as described herein, wherein said therapeutically effective amount is a total daily dosage of about 20 mg to about 2,500 mg.

In some embodiments, provided herein is a method of treating kidney stones in a subject in need thereof, the method including administering to the subject a therapeutically effective amount of Compound 1a or a tautomer thereof including crystalline Form A as described herein or a pharmaceutical composition including Form A as described herein, wherein said therapeutically effective amount is a total daily dosage of about 20 mg to about 2,500 mg.

In yet another aspect, the present disclosure provides a method of preventing the development, worsening, or recurrence of kidney stones in a subject in need thereof, the method including administering to the subject a therapeutically effective amount of Compound 1, having the formula:

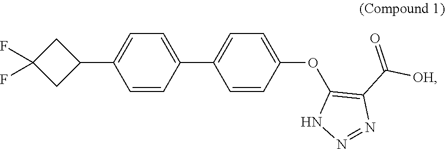 [SMILES: O=C(O)c1nn[nH]c1Oc1ccc(-c2ccc(C3CC(F)(F)C3)cc2)cc1]

a tautomer, a pharmaceutically acceptable salt, a hydrate, or a combination thereof, including one or more of crystalline Forms A, D, E, and C, as described herein, or a pharmaceutical composition including one or more of crystalline Forms A, D, E, and C, as described herein, wherein said therapeutically effective amount is a total daily dosage of about 20 mg to about 2,500 mg.

In some embodiments, provided herein is a method of preventing the development, worsening, or recurrence of kidney stones in a subject in need thereof, the method including administering to the subject a therapeutically effective amount of Compound 1a or a tautomer thereof including crystalline Form A as described herein or a pharmaceutical composition including Form A as described herein, wherein said therapeutically effective amount is a total daily dosage of about 20 mg to about 2,500 mg.

In yet another aspect, the present disclosure provides a method of reducing the size of kidney stones in a subject in need thereof, the method comprising administering to the subject a therapeutically effective amount of Compound 1, having the formula:

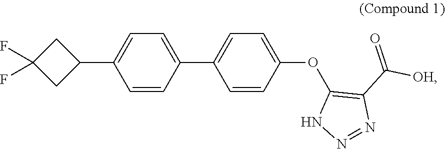 [SMILES: O=C(O)c1nn[nH]c1Oc1ccc(-c2ccc(C3CC(F)(F)C3)cc2)cc1]

a tautomer, a pharmaceutically acceptable salt, a hydrate, or a combination thereof, including one or more of crystalline Forms A, D, E, and C, as described herein, or a pharmaceutical composition including one or more of crystalline Forms A, D, E, and C, as described herein, wherein said therapeutically effective amount is a total daily dosage of about 20 mg to about 2,500 mg.

In some embodiments, provided herein is a method of reducing the size of kidney stones in a subject in need thereof, the method comprising administering to the subject a therapeutically effective amount of Compound 1a or a tautomer thereof including crystalline Form A as described herein or a pharmaceutical composition including Form A as described herein, wherein said therapeutically effective amount is a total daily dosage of about 20 mg to about 2,500 mg.

### V-1: Compound

Compound 1 or a tautomer thereof can include one or more of crystalline Forms A, D, E, and C, as described herein according to Section III. Crystalline Forms.

In some embodiments, the compound or a pharmaceutical composition used in any one of methods as described herein includes: 1) crystalline Form A of Compound 1a or a tautomer thereof; 2) crystalline Form D or E of Compound 1b, or a tautomer and/or hydrate thereof; 3) co-crystalline Form C of Compound 1 and Compound 1b, or a tautomer thereof; or 4) a combination thereof, each of which is represented by the formula:

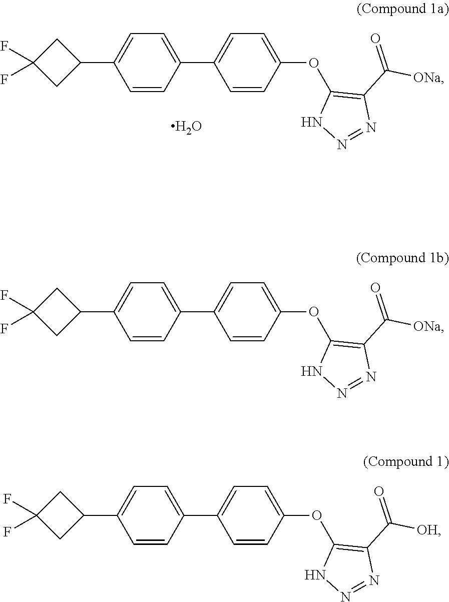

or a tautomer thereof, wherein each of crystalline Forms A, D, E, and C is defined and described herein.

In some embodiments, the compound or a pharmaceutical composition used in any one of methods as described herein includes crystalline Form A of Compound 1a or a tautomer thereof. In some embodiments, the compound used in any one of methods as described herein includes crystalline Form A of Compound 1a or a tautomer thereof. In some embodiments, the pharmaceutical composition used in any one of methods as described herein includes crystalline Form A of Compound 1a or a tautomer thereof.

In some embodiments, the compound or a pharmaceutical composition used in any one of methods as described herein includes Form A of Compound 1a, represented by the formula:

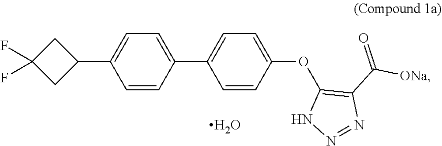

or a tautomer thereof, having the name of sodium 5-((4′-(3,3-difluorocyclobutyl)-[1,1′-biphenyl]-4-yl) oxy)-1H-1,2,3-triazole-4-carboxylate monohydrate or sodium 4-((4′-(3,3-difluorocyclobutyl)-[1,1′-biphenyl]-4-yl)oxy)-1H-1,2,3-triazole-5-carboxylate monohydrate.

In some embodiments, Compound 1a (in Form A) has a purity of at least about 95 area % determined by a high-performance liquid chromatography (HPLC). In some embodiments, Compound 1a (in Form A) has a purity of from about 95 area % to about 100 area %, from about 96 area % to about 100 area %, from about 97 area % to about 100 area %, from about 98 area % to about 100 area %, or from about 99 area % to about 100 area %, determined by a high-performance liquid chromatography (HPLC).

In some embodiments, Compound 1a (in Form A) contains a disodium salt form in an amount of less than about 10%, about 9%, about 8%, about 7%, about 6%, about 5%, about 4%, about 3%, about 2%, or about 1%. In some embodiments, Compound 1a (in Form A) contains a disodium salt form in an amount of less than about 5%, about 4%, about 3%, about 2%, or about 1%. In some embodiments, Compound 1a (in Form A) contains a disodium salt form in an amount of less than about 5%. In some embodiments, Compound 1a (in Form A) contains a disodium salt form in an amount of less than about 4%. In some embodiments, Compound 1a (in Form A) contains a disodium salt form in an amount of less than about 3%. In some embodiments, Compound 1a (in Form A) contains a disodium salt form in an amount of less than about 2%. In some embodiments, Compound 1a (in Form A) contains a disodium salt form in an amount of less than about 1%. In some embodiments, Compound 1a (in Form A) contains a disodium salt form in an amount of from about 0% to 5%, from about 0% to about 4%, from about 0% to about 3%, from about 0% to about 2%, or from about 0% to about 1%.

In some embodiments, Compound 1a (in Form A) contains an acid form (represented by the formula of Compound 1) in an amount of less than about 10%, about 9%, about 8%, about 7%, about 6%, about 5%, about 4%, about 3%, about 2%, or about 1%. In some embodiments, Compound 1a (in Form A) contains an acid form in an amount of less than about 5%, about 4%, about 3%, about 2%, or about 1%. In some embodiments, Compound 1a (in Form A) contains an acid form in an amount of less than about 5%. In some embodiments, Compound 1a (in Form A) contains an acid form in an amount of less than about 4%. In some embodiments, Compound 1a (in Form A) contains an acid form in an amount of less than about 3%. In some embodiments, Compound 1a (in Form A) contains an acid form in an amount of less than about 2%. In some embodiments, Compound 1a (in Form A) contains an acid form in an amount of less than about 1%. In some embodiments, Compound 1a (in Form A) contains an acid form in an amount of from about 0% to 5%, from about 0% to about 4%, from about 0% to about 3%, from about 0% to about 2%, or from about 0% to about 1%.

In some embodiments, Compound 1a (in Form A) contains a disodium salt form and an acid form (represented by the formula of Compound 1) in a combined amount of less than about 10%, about 9%, about 8%, about 7%, about 6%, about 5%, about 4%, about 3%, about 2%, or about 1%. In some embodiments, Compound 1a (in Form A) contains a disodium salt form and an acid form in a combined amount of less than about 5%, about 4%, about 3%, about 2%, or about 1%. In some embodiments, Compound 1a (in Form A) contains a disodium salt form and an acid form in a combined amount of less than about 5%. In some embodiments, Compound 1a (in Form A) contains a disodium salt form and an acid form in a combined amount of less than about 4%. In some embodiments, Compound 1a (in Form A) contains a disodium salt form and an acid form in a combined amount of less than about 3%. In some embodiments, Compound 1a (in Form A) contains a disodium salt form and an acid form in a combined amount of less than about 2%. In some embodiments, Compound 1a (in Form A) contains a disodium salt form and an acid form in a combined amount of less than about 1%. In some embodiments, Compound 1a (in Form A) contains a disodium salt form and an acid form in a combined amount of from about 0% to 5%, from about 0% to about 4%, from about 0% to about 3%, from about 0% to about 2%, or from about 0% to about 1%.

In some embodiments, Compound 1a (in Form A) is substantially free of a disodium salt form and/or an acid form (represented by the formula of Compound 1). In some embodiments, a disodium salt form and/or an acid form is absent in Compound 1a (in Form A). In some embodiments, Compound 1a (in Form A) is substantially free of a disodium salt form. In some embodiments, a disodium salt form is absent in Compound 1a (in Form A). In some embodiments, Compound 1a (in Form A) is substantially free of an acid form. In some embodiments, an acid form is absent in Compound 1a (in Form A). In some embodiments, Compound 1a (in Form A) is substantially free of both a disodium salt form and an acid form.

In some embodiments, Compound 1a (in Form A) is substantially free of one or more crystalline or amorphous forms of Compound 1 represented by the formula:

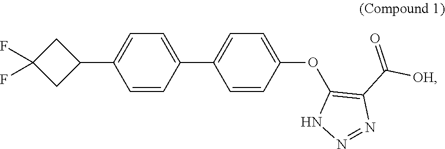 [SMILES: O=C(O)c1nn[nH]c1Oc1ccc(-c2ccc(C3CC(F)(F)C3)cc2)cc1]

a tautomer, a sodium salt, a hydrate, or a combination thereof, provided that the one or more crystalline or amorphous forms of Compound 1 are other than crystalline Form A of Compound 1a. In some embodiments, Compound 1a (in Form A) is substantially free of other crystalline or amorphous forms of Compound 1, 1a, and/or 1b. In some embodiments, Compound 1a (in Form A) is substantially free of other crystalline or amorphous forms of Compound 1a. In some embodiments, Compound 1a (in Form A) is substantially free of other crystalline or amorphous forms of Compound 1, 1a, and 1b.

### V-2: Subject

The subject can have hyperoxaluria and/or kidney stones.

In some embodiments, the subject has hyperoxaluria. In some embodiments, the hyperoxaluria is caused by overproduction of oxalate. In some embodiments, the hyperoxaluria is a primary hyperoxaluria. In some embodiments, the primary hyperoxaluria is primary hyperoxaluria type 1 (PH1). In some embodiments, the subject has primary hyperoxaluria type 1 (PH1).

In some embodiments, the primary hyperoxaluria is primary hyperoxaluria type 2 (PH2) or primary hyperoxaluria type 3 (PH3). In some embodiments, the hyperoxaluria is not caused by a primary hyperoxaluria.

In some embodiments, the subject has primary hyperoxaluria type 1 (PH1) resulting in nephrolithiasis (kidney stones), nephrocalcinosis (deposition of CaOx in the renal parenchyma), and/or progressive chronic kidney disease. In some embodiments, the subject has kidney stones.

In some embodiments, the subject has one or more mutations in an alanine-glyoxylate aminotransferase (AGXT) gene. In some embodiments, the subject has primary hyperoxaluria type 1 (PH1) with one or more mutations in an alanine-glyoxylate aminotransferase (AGXT) gene.

In some embodiments, the subject has a maximal urinary oxalate (uOxalate) excretion of more than about 0.7 mmol/1.73 m² per 24 hours. In some embodiments, the subject has a maximal urinary oxalate (uOxalate) excretion of more than about 0.6 mmol/1.73 m² per 24 hours. In some embodiments, the subject has a maximal urinary oxalate (uOxalate) excretion of more than about 0.46 mmol/1.73 m² per 24 hours.

In some embodiments, the subject has a plasma glycolate (pGlycolate) concentration of less than about 20 μmol/L. In some embodiments, the subject has a plasma glycolate (pGlycolate) concentration of less than about 15 μmol/L. In some embodiments, the subject has a plasma glycolate (pGlycolate) concentration of less than about 10 μmol/L.

In some embodiments, the subject has an estimated glomerular filtration rate (eGFR) of at least about 30 mL/min/1.73 m². In some embodiments, the subject has an estimated glomerular filtration rate (eGFR) of at least about 50 mL/min/1.73 m². In some embodiments, the subject has an estimated glomerular filtration rate (eGFR) of at least about 90 mL/min/1.73 m. In some embodiments, the subject has an estimated glomerular filtration rate (eGFR) of at least about 100 mL/min/1.73 m². The eGFR is estimated using Chronic Kidney Disease Epidemiology Collaboration refit without the race variable equation for a subject, wherein the subject is a human of at least 18 years old and the Bedside Schwartz equation for a subject, wherein the subject is a human of at least 6 years old, but less than 18 years old.

In some embodiments, the subject receives pyridoxine (Vitamin B6) at a stable dose for at least three (3) months prior to the treating and maintains the same dose during the treating. In some embodiments, the subject has a plasma pyridoxine level within a normal range. In some embodiments, the subject has a plasma pyridoxine level within a normal range, prior to the treating. In some embodiments, the subject has a plasma pyridoxine level within a normal range, prior to and during the treating. In some embodiments, the subject receives pyridoxine (Vitamin B6) at a stable dose for at least three (3) months prior to the treating and maintains the same dose during the treating; and the subject has a plasma pyridoxine level within a normal range prior to the treating.

In some embodiments, the subject is other than: 1) a recipient of a liver transplant; 2) who receives a renal replacement therapy selected from the group consisting of hemodialysis, peritoneal dialysis, a continuous renal replacement therapy, or a combination thereof; and/or 3) who is treated with a ribonucleic acid interference (RNAi) agent (e.g., lumasiran) for at least nine (9) months prior to the treating. In some embodiments, the subject is not a recipient of a liver transplant. In some embodiments, the subject does not receive a renal replacement therapy selected from the group consisting of hemodialysis, peritoneal dialysis, a continuous renal replacement therapy, or a combination thereof. In some embodiments, the subject is not treated with a ribonucleic acid interference (RNAi) agent (e.g., lumasiran) for at least nine (9) months prior to the treating. In some embodiments, the RNAi agent is lumasiran.

In some embodiments, the subject is other than: 4) who has received any investigational medicinal product within 30 days or 5 half-lives, whichever is longer, prior to the initiation of the treating; or is in follow-up for another interventional clinical study prior to the treating; and/or 5) who is treated with an experimental RNAi agent (e.g., nedosiran) for at least nine (9) months prior to the treating.

In some embodiments, the subject meets all of inclusion criteria as described in “A Two-Part Multinational, Randomized, Placebo-controlled Trial to Evaluate the Safety and Efficacy of Compound 1a for the Treatment of Children (≥6 years of age) and Adults with Primary Hyperoxaluria Type 1 (a Phase 2/3 Clinical Study)”. In some embodiments, the subject meets all of inclusion criteria as described herein, provided that the subject does not meet any one of exclusion criteria as described herein.

In some embodiments, the subject is a mammal. In some embodiments, the subject is human. In some embodiments, human is an adult of at least 18 years old or an adolescent of at least 12 years old with a body weight of at least 50 kg. In some embodiments, human is a child from 6 to 12 years old or having a body weight of less than 50 kg.

### V-3: Treatment Period

Treatment with Compound 1a including Form A can include Part A treatment (e.g., various groups with a fixed daily dosage of Compound 1a), Part B treatment (e.g., with a daily dosage selected and including a withdrawal period), and/or a long-term extension (LTE) treatment (e.g., with a daily dosage of Compound 1a of Part B). In some embodiments, treatment with Compound 1a is chronic (e.g., lasting at least 1 month, 3 months, 6 months, 9 months, 12 months, 18 months, 2 years, 3 years, or longer, such as the duration of the subject's life).

With reference to Part A treatment, in some embodiments, the treatment includes a single treatment period of Part A (e.g., 28 days), optionally an extension period of Part A (e.g., up to 12 months). In some embodiments, the treatment includes a single treatment period of Part A (e.g., 28 days) and an extension period of Part A (e.g., up to 12 months) after the single treatment period. In some embodiments, the treatment includes an extension period of Part A (e.g., up to 12 months).

In some embodiments, the treatment includes a single treatment period of Part A (e.g., 28 days), optionally an extension period of Part A (e.g., up to 12 months), while a daily dosage of Compound 1a is maintained. In some embodiments, the treatment includes a single treatment period of Part A (e.g., 28 days) and an extension period of Part A (e.g., up to 12 months) after the single treatment period, while a daily dosage of Compound 1a is maintained.

In some embodiments, the single treatment period of Part A has a duration of about 28 days. In some embodiments, the extension period of Part A has a duration of no more than 12 months. In some embodiments, the extension period of Part A has a duration of from 1 to 12 months.

With reference to Part B treatment, in some embodiments, the treatment includes a single treatment period of Part B (e.g., 28 days), a withdrawal period of Part B (e.g., 28 days), and an extension period of Part B (e.g., 28 days), wherein, during the withdrawal period, no compound 1a is administered. In some embodiments, the treatment includes a single treatment period of Part B (e.g., 28 days), a withdrawal period of Part B (e.g., 28 days) after the single treatment period, and an extension period of Part B (e.g., 28 days) after the withdrawal period.

In some embodiments, during Part B treatment, Compound 1a is administered at a daily dosage determined by Part A. In some embodiments, during the single treatment and the extension periods of Part B, Compound 1a is administered at a daily dosage determined by Part A treatment. In some embodiments, during the single treatment and the extension periods of Part B, a daily dosage of Compound 1a is maintained. In some embodiments, during the single treatment and the extension periods of Part B, Compound 1a is administered at a daily dosage determined by Part A and the daily dosage of Compound 1a is maintained.

In some embodiments, the single treatment period of Part B has a duration of about 28 days. In some embodiments, the withdrawal period of Part B has a duration of about 28 days. In some embodiments, the extension period of Part B has a duration of about 28 days.

With reference to the long-term extension (LTE) treatment (e.g., up to 36 months), in some embodiments, Compound 1a is administered at a daily dosage determined by Part B. In some embodiments, Compound 1a is administered at a daily dosage determined by Part B for a duration of from about 1 to about 36 months, from about 6 to about 36 months, from about 12 to about 36 months, or from about 24 to about 36 months. In some embodiments, Compound 1a is administered at a daily dosage determined by Part B for a duration of about 36 months.

With reference to each of Part A, Part B, and LTE treatments, except for the withdrawal period of Part B, Compound 1a is administered daily (e.g., once, twice, three times, or four times daily). In some embodiments, during Part A treatment, Compound 1a is administered daily (e.g., once, twice, three times, or four times daily). In some embodiments, during the single treatment and the extension periods of Part B, Compound 1a is administered daily (e.g., once, twice, three times, or four times daily). In some embodiments, during the LTE treatment, Compound 1a is administered daily (e.g., once, twice, three times, or four times daily).

### III-4: Therapeutically Effective Amount/Administration

In any one of methods as described herein, Compound 1a or a tautomer thereof administered includes crystalline Form A of Compound 1a; and the amount of Compound 1a administered includes the amount of Na salt and monohydrate.

In some embodiments, the therapeutically effective amount is a total daily dosage of from about 5 mg to about 5,000 mg of Compound 1a or a tautomer thereof. In some embodiments, the therapeutically effective amount is a total daily dosage of from about 5 mg to about 5,000 mg, from about 5 mg to about 4,000 mg, from about 5 mg to about 3,000 mg, from about 5 mg to about 2,500 mg, from about 5 mg to about 2,000 mg, from about 5 mg to about 1,500 mg, from about 5 mg to about 1,000 mg, from about 5 mg to about 600 mg, from about 5 mg to about 300 mg, from about 5 mg to about 200 mg, from about 5 mg to about 100 mg, from about 75 mg to about 2,500 mg, from about 75 mg to about 2,000 mg, from about 75 mg to about 1,500 mg, from about 75 mg to about 1,000 mg, from about 75 mg to about 600 mg, from about 75 mg to about 300 mg, or from about 300 mg to about 600 mg of Compound 1a or a tautomer thereof.

In some embodiments, the therapeutically effective amount is a total daily dosage of about 75 mg to about 600 mg of Compound 1a or a tautomer thereof. In some embodiments, the therapeutically effective amount is a total daily dosage of about 75 mg to about 300 mg of Compound 1a or a tautomer thereof. In some embodiments, the therapeutically effective amount is a total daily dosage of about 300 mg to about 600 mg of Compound 1a or a tautomer thereof. In some embodiments, the therapeutically effective amount is a total daily dosage of about 5 mg to about 600 mg of Compound 1a or a tautomer thereof. In some embodiments, the therapeutically effective amount is a total daily dosage of about 5 mg to about 300 mg of Compound 1a or a tautomer thereof. In some embodiments, the therapeutically effective amount is a total daily dosage of about 5 mg to about 200 mg of Compound 1a or a tautomer thereof. In some embodiments, the therapeutically effective amount is a total daily dosage of about 5 mg to about 150 mg of Compound 1a or a tautomer thereof. In some embodiments, the therapeutically effective amount is a total daily dosage of about 5 mg to about 100 mg of Compound 1a or a tautomer thereof.

In some embodiments, the total daily dosage of Compound 1a or a tautomer thereof is about 30 mg to about 60 mg. In some embodiments, the total daily dosage of Compound 1a or a tautomer thereof is about 60 mg to about 90 mg. In some embodiments, the total daily dosage of Compound 1a or a tautomer thereof is about 100 mg to about 140 mg. In some embodiments, the total daily dosage of Compound 1a or a tautomer thereof is about 250 mg to about 500 mg. In some embodiments, the total daily dosage of Compound 1a or a tautomer thereof is about 330 mg to about 370 mg. In some embodiments, the total daily dosage of Compound 1a or a tautomer thereof is about 450 mg to about 550 mg. In some embodiments, the total daily dosage of Compound 1a or a tautomer thereof is about 700 mg to about 800 mg. In some embodiments, the total daily dosage of Compound 1a or a tautomer thereof is about 970 mg to about 1030 mg. In some embodiments, the total daily dosage of Compound 1a or a tautomer thereof is about 1960 mg to about 2040 mg. In some embodiments, the total daily dosage of Compound 1a or a tautomer thereof is about 2060 mg to about 3040 mg.

In some embodiments, the total daily dosage of Compound 1a or a tautomer thereof is about 230 mg to about 270 mg. In some embodiments, the total daily dosage of Compound 1a or a tautomer thereof is about 470 mg to about 530 mg. In some embodiments, the total daily dosage of Compound 1a or a tautomer thereof is about 730 mg to about 780 mg.

In some embodiments, the total daily dosage of Compound 1a or a tautomer thereof is about 20 mg. In some embodiments, the total daily dosage of Compound 1a or a tautomer thereof is about 40 mg. In some embodiments, the total daily dosage of Compound 1a or a tautomer thereof is about 75 mg. In some embodiments, the total daily dosage of Compound 1a or a tautomer thereof is about 120 mg. In some embodiments, the total daily dosage of Compound 1a or a tautomer thereof is about 125 mg. In some embodiments, the total daily dosage of Compound 1a or a tautomer thereof is about 300 mg. In some embodiments, the total daily dosage of Compound 1a or a tautomer thereof is about 360 mg. In some embodiments, the total daily dosage of Compound 1a or a tautomer thereof is about 1000 mg. In some embodiments, the total daily dosage of Compound 1a or a tautomer thereof is about 2000 mg. In some embodiments, the total daily dosage of Compound 1a or a tautomer thereof is about 250 mg. In some embodiments, the total daily dosage of Compound 1a or a tautomer thereof is about 500 mg. In some embodiments, the total daily dosage of Compound 1a or a tautomer thereof is about 600 mg. In some embodiments, the total daily dosage of Compound 1a or a tautomer thereof is about 750 mg.

In some embodiments, the therapeutically effective amount is a total daily dosage of from about 40 mg to about 3000 mg, from about 40 mg to about 2000 mg, from about 40 mg to about 1000 mg, from about 75 mg to about 1000 mg, from about 75 mg to about 800 mg, or from about 75 mg to about 600 mg of Compound 1a or a tautomer thereof, when the subject is an adult of at least 18 years old or an adolescent of at least 12 years old with a body weight of at least 50 kg.

In some embodiments, the therapeutically effective amount is a total daily dosage of about 75 mg, about 300 mg, or about 600 mg of Compound 1a, when the subject is an adult of at least 18 years old or an adolescent of at least 12 years old with a body weight of at least 50 kg. In some embodiments, the therapeutically effective amount is a total daily dosage of about 75 mg of Compound 1a, when the subject is an adult of at least 18 years old or an adolescent of at least 12 years old with a body weight of at least 50 kg. In some embodiments, the therapeutically effective amount is a total daily dosage of about 300 mg of Compound 1a, when the subject is an adult of at least 18 years old or an adolescent of at least 12 years old with a body weight of at least 50 kg. In some embodiments, the therapeutically effective amount is a total daily dosage of about 600 mg of Compound 1a, when the subject is an adult of at least 18 years old or an adolescent of at least 12 years old with a body weight of at least 50 kg.

A person of skill in the art would recognize that an adjustment of a therapeutically effective amount of Compound 1a is necessary, when the subject is a child from 6 to 12 years old or having a body weight of less than 50 kg.

When the subject is a child from 6 to 12 years old or having a body weight of less than 50 kg, in some embodiments, the therapeutically effective amount of Compound 1a is adjusted to about ½, about ⅓, about ¼, or about ⅕ of a total daily dosage as described herein for the subject being an adult of at least 18 years old or an adolescent of at least 12 years old with a body weight of at least 50 kg. In some embodiments, the therapeutically effective amount of Compound 1a is adjusted to about ½ of a total daily dosage as described herein for the subject being an adult of at least 18 years old or an adolescent of at least 12 years old with a body weight of at least 50 kg. In some embodiments, the therapeutically effective amount of Compound 1a is adjusted to about ⅓ of a total daily dosage as described herein for the subject being an adult of at least 18 years old or an adolescent of at least 12 years old with a body weight of at least 50 kg. In some embodiments, the therapeutically effective amount of Compound 1a is adjusted to about ¼ of a total daily dosage as described herein for the subject being an adult of at least 18 years old or an adolescent of at least 12 years old with a body weight of at least 50 kg. In some embodiments, the therapeutically effective amount of Compound 1a is adjusted to about ⅕ of a total daily dosage as described herein for the subject being an adult of at least 18 years old or an adolescent of at least 12 years old with a body weight of at least 50 kg.

In general, Compound 1a can be administered orally. In some embodiments, Compound 1a or a tautomer thereof is administered orally. In some embodiments, Compound 1a or a tautomer thereof including Form A in a tablet formulation is administered orally.

Compound 1a or a tautomer thereof can be administered once (e.g., q.d.), twice (e.g., b.i.d. or q12h), three (e.g., t.i.d.), or four times (e.g., q.i.d.) a day. In some embodiments, Compound 1a or a tautomer thereof is administered once daily (i.e., QD). In some embodiments, Compound 1a or a tautomer thereof is administered twice daily (i.e., BID), such as in two doses separated by approximately 12 hours (i.e., Q12H). In some embodiments, Compound 1a or a tautomer thereof is administered three times daily. In some embodiments, Compound 1a or a tautomer thereof is administered four times daily. In some embodiments, Compound 1a or a tautomer thereof is administered once every other day (i.e., QOD).

In some embodiments, about 40 mg of Compound 1a or a tautomer thereof is administered once daily. In some embodiments, about 75 mg of Compound 1a or a tautomer thereof is administered once daily. In some embodiments, about 120 mg of Compound 1a or a tautomer thereof is administered once daily. In some embodiments, about 300 mg of Compound 1a or a tautomer thereof is administered once daily. In some embodiments, about 360 mg of Compound 1a or a tautomer thereof is administered once daily. In some embodiments, about 500 mg of Compound 1a or a tautomer thereof is administered once daily. In some embodiments, about 600 mg of Compound 1a or a tautomer thereof is administered once daily. In some embodiments, about 750 mg of Compound 1a or a tautomer thereof is administered once daily. In some embodiments, about 1000 mg of Compound 1a or a tautomer thereof is administered once daily. In some embodiments, about 2000 mg of Compound 1a or a tautomer thereof is administered once daily.

In some embodiments, about 75 mg of Compound 1a or a tautomer thereof including Form A is administered once daily. In some embodiments, about 300 mg of Compound 1a or a tautomer thereof including Form A is administered once daily. In some embodiments, about 600 mg of Compound 1a or a tautomer thereof including Form A is administered once daily.

In some embodiments, about 35 mg of Compound 1a or a tautomer thereof is administered twice daily. In some embodiments, about 125 mg of Compound 1a or a tautomer thereof is administered twice daily. In some embodiments, about 150 mg of Compound 1a or a tautomer is administered twice daily. In some embodiments, about 300 mg of Compound 1a or a tautomer is administered twice daily. In some embodiments, about 500 mg of Compound 1a or a tautomer is administered twice daily. In some embodiments the twice daily dosages are administered about 12 hours apart.

In some embodiments, Compound 1a or a tautomer thereof is administered for at least 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83 84, 85, 86, 87, 88, 89, 90, 91, 92, 93, 94, 95, 96, 97, 98, 99, 100 days, or longer. In some embodiments, Compound 1a or a tautomer thereof is administered for 7, 14, 21, 28, 35, 42, 49, or 56 days. In some embodiments, Compound 1a or a tautomer thereof is administered for 28 days. In some embodiments, Compound 1a or a tautomer thereof is administered for 56 days. In some embodiments, Compound 1a or a tautomer thereof is administered for 84 days.

In some embodiments, Compound 1a or a tautomer thereof is administered for 28 days during each of the single treatment period of Part A, the single treatment period of Part B, and the extension period of Part B, as described in Section V-3. Treatment Period.

In some embodiments, Compound 1a or a tautomer thereof is administered for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more months. In some embodiments, Compound 1a or a tautomer thereof is administered for at least 10, 15, 20, 25, 30, 35, 40, 45, or 50 months. In some embodiments, Compound 1a or a tautomer thereof is administered for at least 6 months. In some embodiments, Compound 1a or a tautomer thereof is administered for at least 12 months. In some embodiments, Compound 1a or a tautomer thereof is administered for at least 18 months. In some embodiments, Compound 1a or a tautomer thereof is administered for at least 24 months. In some embodiments, Compound 1a or a tautomer thereof is administered for at least 30 months. In some embodiments, Compound 1a or a tautomer thereof is administered for at least 36 months. In some embodiments, Compound 1a or a tautomer thereof is administered for at least 42 months. In some embodiments, Compound 1a or a tautomer thereof is administered for the lifetime of the subject.

In some embodiments, Compound 1a or a tautomer thereof is administered for about 12 months during the extension period of Part A treatment as described herein. In some embodiments, Compound 1a or a tautomer thereof is administered for about 36 months during the long-term extension (LTE) period of the treatment as described in Section V-3. Treatment Period.

### V-5: Efficacy

A Two-Part Multinational, Randomized, Placebo-controlled Trial can evaluate the safety and efficacy of Compound 1a for the treatment of children (≥6 years of age) and adults with primary hyperoxaluria type 1 (PH1).

Administration of a therapeutically effective amount of Compound 1a may reduce or substantially eliminate one or more symptoms associated with primary hyperoxaluria type 1 (PH1) in subjects.

In some embodiments, the therapeutically effective amount of Compound 1a or a tautomer thereof reduces the uOxalate excretion to less than about 0.46 mmol/1.73 m² per 24 hours. In some embodiments, the therapeutically effective amount of Compound 1a or a tautomer thereof including Form A reduces the uOxalate excretion to less than about 0.46 mmol/1.73 m² per 24 hours.

In some embodiments, the therapeutically effective amount of Compound 1a or a tautomer thereof reduces the uOxalate excretion by at least about 50% relative to a uOxalate excretion prior to the treating. In some embodiments, the therapeutically effective amount of Compound 1a or a tautomer thereof including Form A reduces the uOxalate excretion by from about 50% relative to a uOxalate excretion prior to the treating.

In some embodiments, the therapeutically effective amount of Compound 1a or a tautomer thereof brings a ratio of uOxalate to urine creatinine (uCreatinine) to a normal range of less than about 63.7 mg/mg, which is 80 mmol/mmol. In some embodiments, the therapeutically effective amount of Compound 1a or a tautomer thereof including Form A brings a ratio of uOxalate to urine creatinine (uCreatinine) to a normal range of less than about 63.7 mg/mg, which is 80 mmol/mmol.

In some embodiments, the therapeutically effective amount of Compound 1a or a tautomer thereof reduces a plasma glycolate (pGlycolate) concentration in an absolute amount and/or in a relative amount compared to a pGlycolate concentration prior to the treating. In some embodiments, the therapeutically effective amount of Compound 1a or a tautomer thereof including Form A reduces a plasma glycolate (pGlycolate) concentration in an absolute amount and/or in a relative amount compared to a pGlycolate concentration prior to the treating.

In some embodiments, the therapeutically effective amount of Compound 1a or a tautomer thereof increases an estimated glomerular filtration rate (eGFR) in an absolute amount and/or in a relative amount compared to an eGFR prior to the treating. In some embodiments, the therapeutically effective amount of Compound 1a or a tautomer thereof including Form A increases an estimated glomerular filtration rate (eGFR) in an absolute amount and/or in a relative amount compared to an eGFR prior to the treating.

In some embodiments, the therapeutically effective amount of Compound 1a or a tautomer thereof reduces a size of the kidney stones. In some embodiments, the therapeutically effective amount of Compound 1a or a tautomer thereof including Form A reduces a size of the kidney stones.

In some embodiments, the therapeutically effective amount of Compound 1a or a tautomer thereof prevents the development, worsening, or recurrence of kidney stones. In some embodiments, the therapeutically effective amount of Compound 1a or a tautomer thereof including Form A prevents the development, worsening, or recurrence of kidney stones.

In some embodiments, the subject is further evaluated to by one or more tests to provide overall assessments including plasma pharmacokinetic and/or pharmacodynamic profiles.

### VI. Kits

The disclosure also encompasses kits including pharmaceutical compositions and dosage forms including one or more of crystalline Forms A, D, E, and C, as described herein.

In some aspects, the present disclosure provides a kit that includes a pharmaceutical composition and dosage form including one or more of crystalline Forms A, D, E, and C, as described herein. In some embodiments, the present disclosure provides a kit that includes a pharmaceutical composition and dosage form including Form A of Compound 1a as described herein (see Section III-1. Crystalline Form A of Compound 1a). Some of the kits described herein include a label describing a method of administering Compound 1, Compound 1a, Compound 1b, a tautomer thereof, or a combination thereof. In some embodiments, kits described herein include a label describing a method of administering Compound 1a or a tautomer thereof. Some of the kits described herein include a label describing a method of treating hyperoxaluria. In some embodiments, the kits described herein include a label describing a method of treating kidney stones. In some embodiments, the kit includes a pharmaceutical composition and dosage form including Form A of Compound 1a as described herein and a label describing a method of administering Compound 1a or a tautomer thereof. In some embodiments, the kit includes a pharmaceutical composition and dosage form including Form A of Compound 1a as described herein and a label describing a method of treating hyperoxaluria. In some embodiments, the kit includes a pharmaceutical composition and dosage form including Form A of Compound 1a as described herein and a label describing a method of treating kidney stones.

The compositions of the present disclosure, including but not limited to, compositions including one or more of crystalline Forms A, D, E, and C, as described herein in a bottle, jar, vial, ampoule, tube, blister pack, or other container-closure system approved by the Food and Drug Administration (FDA) or other regulatory body, which may provide one or more dosages containing the compounds. In some embodiments, the composition includes Form A of Compound 1a or a tautomer thereof. The package or dispenser may also be accompanied by a notice associated with the container in a form prescribed by a governmental agency regulating the manufacture, use, or sale of pharmaceuticals, the notice indicating approval by the agency. In certain aspects, the kit may include a formulation or composition as described herein, a container closure system including the formulation or a dosage unit form including the formulation, and a notice or instructions describing a method of use as described herein.

### VII. Processes

In one aspect, the present disclosure provides a process for preparing a compound represented by formula (I):

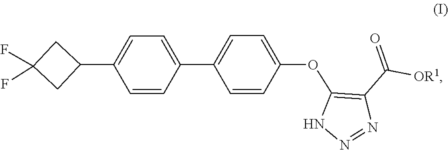

or a tautomer and/or a salt thereof via Steps a) and b). The method includes:
a) combining a compound of formula (IV):

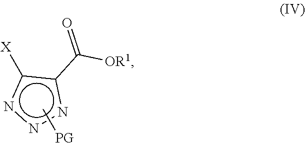

or a salt thereof, a compound of formula (III):

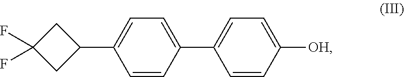 [SMILES: Oc1ccc(-c2ccc(C3CC(F)(F)C3)cc2)cc1]

or a salt thereof, and a first base in a first solvent to form a compound of formula (II):

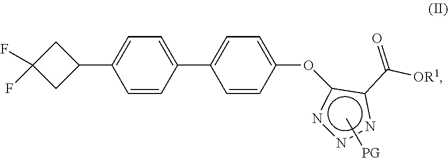

or a salt thereof; and
b) removing the PG group of the compound of formula (II) or the salt thereof to provide a compound of formula (I):

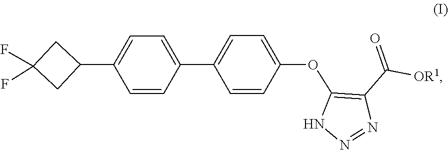

or a tautomer and/or a salt thereof,
wherein:
R¹ is C₁₋₆ alkyl;
X in formula (IV) is halogen or a sulfonate;

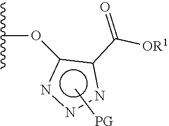

in formula (IV) or (II) is represented by the formula:

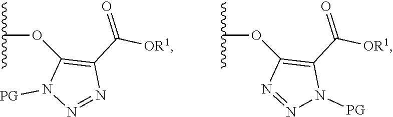

or a mixture thereof; and
PG is an amine-protecting group.

With reference to formula (II) or (IV), the 1,2,3-triazole moiety is protected with an amine-protecting group (PG). Suitable amine-protecting group include, but are not limited to, a benzyl (Bn) group, 2-(trimethylsilyl)ethoxymethyl (SEM), p-methoxybenzyl (PMB) (also known as 4-methoxybenzyl), 2,4-dimethoxybenzyl (DMB), 3,4-dimethoxybenzyl (DMPM), 1-(2,4-dimethoxyphenyl)ethyl, p-methoxyphenyl (PMP), p-toluenesulfonyl (Ts), and p-nitrophenylsulfonyl (a Nosyl group). In some embodiments, the amine-protecting group is 2-(trimethylsilyl)ethoxymethyl, 4-methoxybenzyl, 2,4-dimethoxybenzyl, 3,4-dimethoxybenzyl, or 1-(2,4-dimethoxyphenyl)ethyl. In some embodiments, the amine-protecting group is 2-(trimethylsilyl)ethoxymethyl, 4-methoxybenzyl, or 2,4-dimethoxybenzyl. In some embodiments, the amine-protecting group is 4-methoxybenzyl.

In some embodiments, the compound of formula (IV) is represented by:

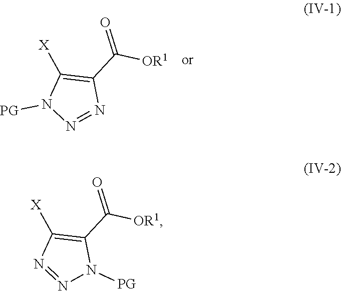

or a mixture thereof; and the compound of formula (II) is represented by:

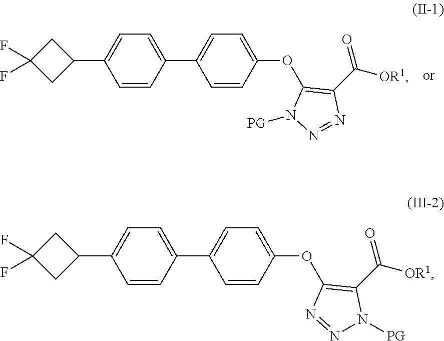

or a mixture thereof, wherein R¹ and X are as defined and described herein.

In some embodiments, when the amine-protecting group is benzyl (Bn), p-methoxybenzyl (PMB), 2,4-dimethoxybenzyl group (DMB), 3,4-dimethoxybenzyl (DMPM), or 1-(2,4-dimethoxyphenyl)ethyl, the compound of formula (IV) is represented by formula (IV-1):

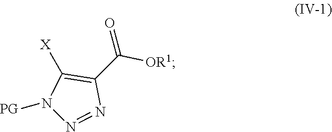

and
the compound of formula (II) is represented by formula (II-1):

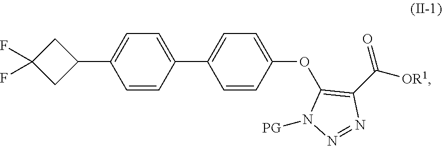

wherein R¹ and X are as defined and described herein.

With reference to any one of formulae (I), (II), and (IV), and their related formulae, in some embodiments, R¹ is methyl, ethyl, n-propyl, isopropyl, n-butyl, isobutyl, sec-butyl, tert-butyl, n-pentyl, isopentyl, or n-hexyl. In some embodiments, R¹ is methyl. In some embodiments, R¹ is ethyl.

With reference to formula (IV), and its related formulae, in some embodiments, X is halo. In some embodiments, X is Cl, Br, or I. In some embodiments, X is a sulfonate. In some embodiments, X is OMs, OTs, or OTf. In some embodiments, X is Cl, Br, I, OMs, OTs, or OTf.

In some embodiments, X is Cl.

In some embodiments, the compound of formula (IV) is represented by:

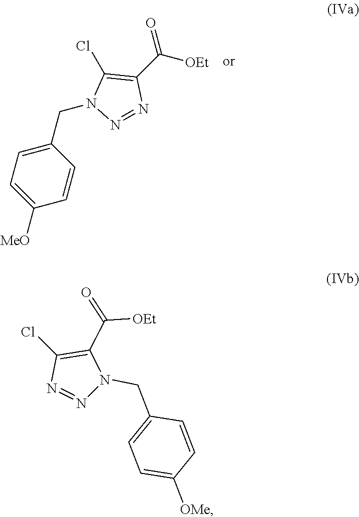 [SMILES: CCOC(=O)c1nnn(Cc2ccc(OC)cc2)c1Cl ; "or" ; CCOC(=O)c1c(Cl)nnn1Cc1ccc(OC)cc1]

or a mixture thereof.

In some embodiments, the compound of formula (IV) is represented by formula (IVa):

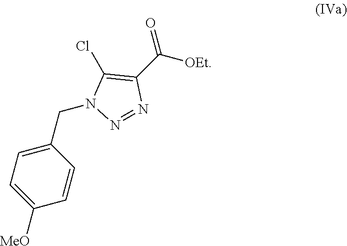 [SMILES: CCOC(=O)c1nnn(Cc2ccc(OC)cc2)c1Cl]

In some embodiments, the compound of formula (II) is represented by:

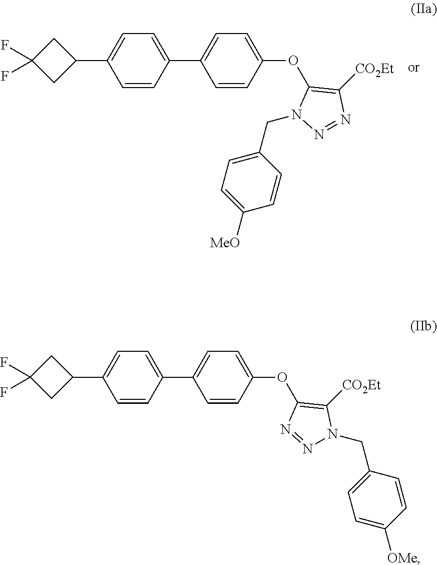 [SMILES: CCOC(=O)c1nnn(Cc2ccc(OC)cc2)c1Oc1ccc(-c2ccc(C3CC(F)(F)C3)cc2)cc1 ; "or" ; CCOC(=O)c1c(Oc2ccc(-c3ccc(C4CC(F)(F)C4)cc3)cc2)nnn1Cc1ccc(OC)cc1]

or a mixture thereof.

In some embodiments, the compound of formula (II) is represented by formula (IIa):

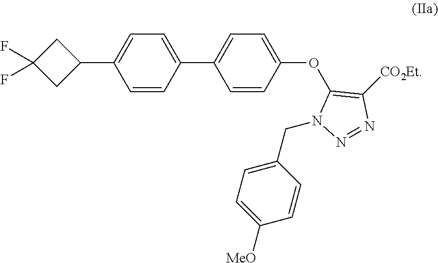 [SMILES: CCOC(=O)c1nnn(Cc2ccc(OC)cc2)c1Oc1ccc(-c2ccc(C3CC(F)(F)C3)cc2)cc1]

In some embodiments, the compound of formula (IV) is represented by formula (IVa):

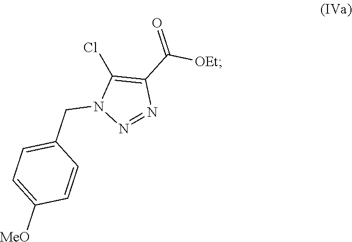 [SMILES: CCOC(=O)c1nnn(Cc2ccc(OC)cc2)c1Cl]

the compound of formula (II) is represented by formula (IIa):

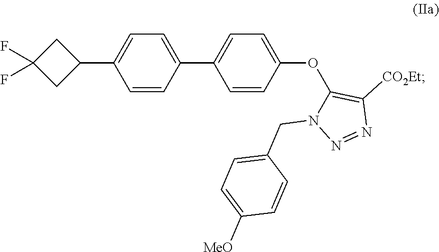 [SMILES: CCOC(=O)c1nnn(Cc2ccc(OC)cc2)c1Oc1ccc(-c2ccc(C3CC(F)(F)C3)cc2)cc1]

and
the compound of formula (I) is represented by formula (Ia):

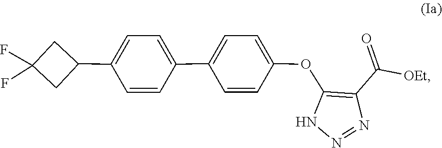 [SMILES: CCOC(=O)c1nn[nH]c1Oc1ccc(-c2ccc(C3CC(F)(F)C3)cc2)cc1]

or a tautomer and/or a salt thereof.

### Step a)

With reference to step a), the first base can be an alkali base (e.g., an alkali carbonate, an alkali phosphate tribasic, and an alkali carboxylate), an amine (e.g., a tertiary amine or an aromatic amine), an amidine-based compound, or a combination thereof. Suitable alkali carbonates include lithium carbonate, sodium carbonate, potassium carbonate, and cesium carbonate. Suitable alkali phosphates tribasic include sodium phosphate tribasic and potassium phosphate tribasic. Suitable alkali carboxylates include lithium acetate, sodium acetate, potassium acetate, cesium acetate, and potassium trimethylacetate. Suitable amines tertiary amines and aromatic amine bases. Suitable amidine-based compounds include 1,8-diazabicyclo[5.4.0]undec-7-ene (DBU) and 1,5-diazabicyclo[4.3.0]non-5-en (DBN). In some embodiments, the first base is:
i) an alkali base selected from the group consisting of sodium carbonate, potassium carbonate, cesium carbonate, sodium phosphate tribasic, potassium phosphate tribasic, sodium acetate, potassium acetate, and cesium acetate;
ii) a tertiary amine selected from the group consisting of triethylamine, tri-n-butylamine, N,N-diisopropylethylamine, N-methylpyrrolidine, N-methylmorpholine, dimethylaniline, diethylaniline, 1,8-bis(dimethylamino)naphthalene, quinuclidine, and 1,4-diazabicylo[2.2.2]-octane (DABCO);
iii) an aromatic amine base selected from the group consisting of pyridine, lutidines, collidines, 4-dimethylaminopyridine, imidazole, dimethylaniline, and diethylaniline;
iv) an amidine-based compound selected from the group consisting of 1,8-diazabicyclo[5.4.0]undec-7-ene (DBU) and 1,5-diazabicyclo[4.3.0]non-5-en (DBN); or
v) a combination thereof.

In some embodiments of step a), the first base is an amidine-based compound selected from the group consisting of 1,8-diazabicyclo[5.4.0]undec-7-ene (DBU) and 1,5-diazabicyclo[4.3.0]non-5-en (DBN). In some embodiments, the first base includes 1,8-diazabicyclo[5.4.0]undec-7-ene (DBU). In some embodiments, the first base is 1,8-diazabicyclo[5.4.0]undec-7-ene (DBU).

In some embodiments of step a), the first base is an alkali carbonate selected from the group consisting of sodium carbonate, potassium carbonate, and cesium carbonate. In some embodiments, the first base includes cesium carbonate. In some embodiments, the first base is cesium carbonate.

In some embodiments of step a), the first base is an alkali acetate selected from the group consisting of sodium acetate, potassium acetate, and cesium acetate. In some embodiments, the first base includes cesium acetate. In some embodiments, the first base is cesium acetate.

In general, the compound of formula (III) can be in a stoichiometrical amount (e.g., about 1 equivalent) or stoichiometrically excess (e.g., more than 1 equivalent), relative to the compound of formula (IV) or any one of its related formulae. In some embodiments, the compound of formula (III) is in an amount of at least about 1.0 equivalent, relative to the compound of formula (IV) or any one of its related formulae. In some embodiments, the compound of formula (III) is in an amount of no more than about 2.0 equivalents, relative to the compound of formula (IV) or any one of its related formulae. In some embodiments, the compound of formula (III) is in an amount of at least about 1.0 equivalent, relative to the compound of formula (IVa). In some embodiments, the compound of formula (III) is in an amount of no more than about 2.0 equivalents, relative to the compound of formula (IVa).

In some embodiments, the compound of formula (III) is in an amount of from about 1.0 to about 2.0 equivalents, relative to the compound of formula (IV) or any one of its related formulae. In some embodiments, the compound of formula (III) is in an amount of from about 1.0 to about 1.5 equivalents, from about 1.0 to about 1.4 equivalents, from about 1.0 to about 1.3 equivalents, from about 1.0 to about 1.2 equivalents, from about 1.0 to about 1.1 equivalents, or about 1.0 equivalent, relative to the compound of formula (IV) or any one of its related formulae. In some embodiments, the compound of formula (III) is in an amount of from about 1.0 to about 1.2 equivalents, from about 1.0 to about 1.1 equivalents, or about 1.0 equivalent, relative to the compound of formula (IV) or any one of its related formulae. In some embodiments, the compound of formula (III) is in an amount of from about 1.0 to about 1.1 equivalents, relative to the compound of formula (IV) or any one of its related formulae. In some embodiments, the compound of formula (III) is in an amount of about 1.0 equivalent, relative to the compound of formula (IV) or any one of its related formulae. In some embodiments, the compound of formula (III) is in an amount of from about 1.0 to about 1.5 equivalents, from about 1.0 to about 1.4 equivalents, from about 1.0 to about 1.3 equivalents, from about 1.0 to about 1.2 equivalents, from about 1.0 to about 1.1 equivalents, or about 1.0 equivalent, relative to the compound of formula (IVa). In some embodiments, the compound of formula (III) is in an amount of from about 1.0 to about 1.2 equivalents, from about 1.0 to about 1.1 equivalents, or about 1.0 equivalent, relative to the compound of formula (IVa). In some embodiments, the compound of formula (III) is in an amount of from about 1.0 to about 1.1 equivalents, relative to the compound of formula (IVa). In some embodiments, the compound of formula (III) is in an amount of about 1.0 equivalent, relative to the compound of formula (IVa).

In general, the first base can be stoichiometrically excess (e.g., more than 1 equivalent), relative to the compound of formula (IV) or any one of its related formulae. In some embodiments, the first base is in an amount of at least about 1.0 equivalent, relative to the compound of formula (IV) or any one of its related formulae. In some embodiments, the first base is in an amount of no more than about 5, about 4, about 3, or about 2 equivalents, relative to the compound of formula (IV) or any one of its related formulae. In some embodiments, the first base is in an amount of no more than about 2.0 equivalent, relative to the compound of formula (IV) or any one of its related formulae. In some embodiments, the first base is in an amount of at least about 1.0 equivalent, relative to the compound of formula (IVa). In some embodiments, the first base is in an amount of no more than about 3.0 equivalents, relative to the compound of formula (IVa). In some embodiments, the first base is in an amount of no more than about 2.0 equivalents, relative to the compound of formula (IVa).

In some embodiments, the first base is in an amount of from about 1.0 to about 2.0 equivalents, from about 1.0 to about 1.5 equivalents, or about 1.35 equivalents, relative to the compound of formula (IV) or any one of its related formulae. In some embodiments, the first base is in an amount of from about 1.0 to about 1.5 equivalents or about 1.35 equivalents, relative to the compound of formula (IV) or any one of its related formulae. In some embodiments, the first base is in an amount of from about 1.0 to about 1.5 equivalents, relative to the compound of formula (IV) or any one of its related formulae. In some embodiments, the first base is in an amount of about 1.35 equivalents, relative to the compound of formula (IV) or any one of its related formulae. In some embodiments, the first base is in an amount of from about 1.0 to about 2.0 equivalents, from about 1.0 to about 1.5 equivalents, or about 1.35 equivalents, relative to the compound of formula (IVa). In some embodiments, the first base is in an amount of from about 1.0 to about 1.5 equivalents or about 1.35 equivalents, relative to the compound of formula (IVa). In some embodiments, the first base is in an amount of from about 1.0 to about 1.5 equivalents, relative to the compound of formula (IVa). In some embodiments, the first base is in an amount of about 1.35 equivalents, relative to the compound of formula (IVa).

In some embodiments, the compound of formula (III) is in an amount of from about 1.0 to about 1.1 equivalents and the first base is in an amount of from about 1.0 to about 1.5 equivalents, relative to the compound of formula (IV) or any one of its related formulae. In some embodiments, the compound of formula (III) is in an amount of about 1.0 equivalent and the first base is in an amount of about 1.35 equivalents, relative to the compound of formula (IV) or any one of its related formulae. In some embodiments, the compound of formula (III) is in an amount of from about 1.0 to about 1.1 equivalents and the first base is in an amount of from about 1.0 to about 1.5 equivalents, relative to the compound of formula (IVa). In some embodiments, the compound of formula (III) is in an amount of about 1.0 equivalent and the first base is in an amount of about 1.35 equivalents, relative to the compound of formula (IVa).

The first solvent can be any suitable polar or non-polar aprotic solvent. In some embodiments, the first solvent is benzene, toluene, dioxane, acetone, ethyl acetate (EtOAc), tetrahydrofuran (THF), 2-methyl-tetrahydrofuran (MeTHF), acetonitrile (ACN), N-methylpyrrolidone (NMP), N,N-dimethylformamide (DMF), N,N-dimethylacetamide (DMAC), dimethoxyethane (DME), dimethyl sulfoxide (DMSO), propylene carbonate (PC), or a combination thereof. In some embodiments, the first solvent includes N,N-dimethylacetamide (DMAC), N-methylpyrrolidone (NMP), or a combination thereof. In some embodiments, the first solvent includes N,N-dimethylacetamide (DMAC) or N-methylpyrrolidone (NMP). In some embodiments, the first solvent includes N,N-dimethylacetamide (DMAC). In some embodiments, the first solvent is N,N-dimethylacetamide (DMAC). In some embodiments, the first solvent includes N-methylpyrrolidone (NMP). In some embodiments, the first solvent is N-methylpyrrolidone (NMP).

In some embodiments, first base includes 1,8-diazabicyclo[5.4.0]undec-7-ene (DBU) and the first solvent includes N,N-dimethylacetamide (DMAC); or the first base includes cesium carbonate and the first solvent includes N-methylpyrrolidone (NMP). In some embodiments, first base includes 1,8-diazabicyclo[5.4.0]undec-7-ene (DBU) and the first solvent includes N,N-dimethylacetamide (DMAC). In some embodiments, the first base includes cesium carbonate and the first solvent includes N-methylpyrrolidone (NMP). In some embodiments, first base is 1,8-diazabicyclo[5.4.0]undec-7-ene (DBU) and the first solvent is N,N-dimethylacetamide (DMAC). In some embodiments, the first base is cesium carbonate and the first solvent is N-methylpyrrolidone (NMP).

In some embodiments, the compound of formula (III) is in an amount of from about 1.0 to about 1.1 equivalents and 1,8-diazabicyclo[5.4.0]undec-7-ene (DBU) is in an amount of from about 1.0 to about 1.5 equivalents, relative to the compound of formula (IVa). In some embodiments, the compound of formula (III) is in an amount of about 1.0 equivalent and 1,8-diazabicyclo[5.4.0]undec-7-ene (DBU) is in an amount of about 1.35 equivalents, relative to the compound of formula (IVa). In some embodiments, the compound of formula (III) is in an amount of from about 1.0 to about 1.1 equivalents and 1,8-diazabicyclo[5.4.0]undec-7-ene (DBU) is in an amount of from about 1.0 to about 1.5 equivalents, relative to the compound of formula (IVa); and the first solvent includes N,N-dimethylacetamide (DMAC). In some embodiments, the compound of formula (III) is in an amount of about 1.0 equivalent and 1,8-diazabicyclo[5.4.0]undec-7-ene (DBU) is in an amount of about 1.35 equivalents, relative to the compound of formula (IVa); and the first solvent includes N,N-dimethylacetamide (DMAC).

In some embodiments, the compound of formula (III) is in an amount of from about 1.0 to about 1.1 equivalents and cesium carbonate is in an amount of from about 1.0 to about 1.5 equivalents, relative to the compound of formula (IVa). In some embodiments, the compound of formula (III) is in an amount of about 1.0 equivalent and cesium carbonate is in an amount of about 1.35 equivalents, relative to the compound of formula (IVa). In some embodiments, the compound of formula (III) is in an amount of from about 1.0 to about 1.1 equivalents and cesium carbonate is in an amount of from about 1.0 to about 1.5 equivalents, relative to the compound of formula (IVa); and the first solvent is N-methylpyrrolidone (NMP). In some embodiments, the compound of formula (III) is in an amount of about 1.0 equivalent and cesium carbonate is in an amount of about 1.35 equivalents, relative to the compound of formula (IVa); and the first solvent is N-methylpyrrolidone (NMP).

In general, step a) can be conducted at an ambient to an elevated temperature. In some embodiments, step a) is conducted at a temperature of at least about 60° C. In some embodiments, step a) is conducted at a temperature of from about 70° C. to about 110° C., from about 80° C. to about 100° C., or from about 85° C. to about 90° C. In some embodiments, step a) is conducted at a temperature of from about 70° C. to about 110° C. In some embodiments, step a) is conducted at a temperature of from about 80° C. to about 100° C. In some embodiments, step a) is conducted at a temperature of from about 85° C. to about 90° C.

### Step b)

With respect to step b), the compound of formula (II) can be deprotected by various methods to provide the compound of formula (I). When PG is p-methoxybenzyl (PMB), the compound of formula (II) or any one of its related formulae can be deprotected by various methods, for example, under acidic, reductive (hydrogenolysis), or oxidative conditions to provide the compound of the compound of formula (I).

In some embodiments, the PG group is removed by treating with a first acid. In some embodiments, the p-methoxybenzyl (PMB) group is removed by treating with a first acid. In some embodiments, the first acid is trifluoroacetic acid, trichloroacetic acid, hydrochloric acid, sulfuric acid, phosphoric acid, or a combination thereof. In some embodiments, the first acid includes trifluoroacetic acid. In some embodiments, the first acid is trifluoroacetic acid.

Step b) can be conducted in a solvent in addition to the first acid or in the first acid that is also a solvent. In some embodiments, step b) is conducted with the first acid as described herein in a second solvent. The second solvent can be any suitable polar or non-polar, protic or aprotic solvent. In some embodiments, the second solvent is water, C₁₋₄ alcohol, ethyl acetate, isopropyl acetate, butyl acetate, isobutyl acetate, tetrahydrofuran, 2-methyltetrahydrofuran, dioxane, benzene, toluene, xylenes, chlorobenzene, dichloromethane, 1,2-dichloroethane, or a combination thereof. In some embodiments, the second solvent is dichloromethane or 1,2-dichloroethane. In some embodiments, the second solvent includes dichloromethane. In some embodiments, the second solvent is dichloromethane. In some embodiments, step b) is conducted in the first acid as described herein. In some embodiments, step b) is conducted in trifluoroacetic acid.

When the PG group is removed under the acidic conditions, in some embodiments, the deprotection reaction mixture further includes a transferring agent. When PG is p-methoxybenzyl (PMB), in some embodiments, the transferring agent is anisole. When PG is p-methoxybenzyl (PMB), in some embodiments, the second solvent includes anisole.

In some embodiments, the p-methoxybenzyl (PMB) group is removed by treating with trifluoroacetic acid and anisole in dichloromethane. In some embodiments, the p-methoxybenzyl (PMB) group is removed by treating with trifluoroacetic acid and anisole.

In general, step b) under the acidic conditions can be performed at an ambient to an elevated temperature. For example, the reaction mixture of step b) can be at a temperature of from about 30° C. to about 70° C. In some embodiments, step b) is conducted at a temperature of from about 50° C. to about 70° C. In some embodiments, step b) is conducted at a temperature of from about 50° C. to about 60° C. In some embodiments, step b) is conducted at a temperature of from about 55° C. to about 60° C. in trifluoroacetic acid and anisole.

In some embodiments, the p-methoxybenzyl (PMB) group is removed by hydrogenolysis. In some embodiments, the p-methoxybenzyl (PMB) group is removed under oxidative conditions by treating 2,3-dichloro-5,6-dicyano-1,4-benzoquinone (DDQ).

### Steps c) and d)

In some embodiments, the process further includes:
c) combining the compound of formula (I), or the tautomer and/or the salt thereof, with a second base in a third solvent; and
d) acidifying with a second acid to provide Compound 1 represented by the formula:

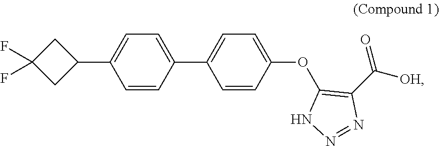 [SMILES: O=C(O)c1nn[nH]c1Oc1ccc(-c2ccc(C3CC(F)(F)C3)cc2)cc1]

a tautomer, a salt, a hydrate, or a combination thereof.

With reference to step c), the second base can be an alkali carbonate, an alkali hydroxide, an alkali alkoxide, or a combination thereof. Suitable alkali carbonates include lithium carbonate, sodium carbonate, potassium carbonate, and cesium carbonate. Suitable alkali hydroxides include lithium hydroxide, sodium hydroxide, potassium hydroxide, and cesium hydroxide. Suitable alkali alkoxides include sodium methoxide, sodium tert-butoxide, and potassium tert-butoxide. In some embodiments, the second base is lithium carbonate, sodium carbonate, potassium carbonate, cesium carbonate, lithium hydroxide, sodium hydroxide, potassium hydroxide, cesium hydroxide, sodium methoxide, sodium tert-butoxide, potassium tert-butoxide, or a combination thereof. In some embodiments, the second base is lithium hydroxide, sodium hydroxide, or potassium hydroxide. In some embodiments, the second base is sodium hydroxide. In some embodiments, the second base is an aqueous solution of sodium hydroxide.

The third solvent can be any suitable polar or non-polar, protic, or aprotic solvent. In some embodiments, the third solvent includes water. In some embodiments, the third solvent is water.

In some embodiments, the step d) is conducted in an aqueous solution. In some embodiments, step c) is conducted with sodium hydroxide in water.

In general, step c) can be performed at an ambient to an elevated temperature. For example, the reaction mixture of step c) can be at a temperature of from 30° C. to 100° C. or heated to reflux. In some embodiments, step c) is conducted at a temperature of from about 40° C. to about 100° C., from about 50° C. to about 90° C., from about 60° C. to about 90° C., from about 70° C. to about 90° C., or from about 75° C. to about 85° C. In some embodiments, step c) is conducted at a temperature of from about 75° C. to about 85° C.

With reference to step d), in some embodiments, the second acid is hydrochloric acid, sulfuric acid, phosphoric acid, or a combination thereof. In some embodiments, the second acid is hydrochloric acid. In some embodiments, the second acid is an aqueous solution of hydrochloric acid.

In some embodiments, the step d) is conducted in an aqueous solution. In some embodiments, the step d) is conducted by treating an aqueous extract of step c) with an aqueous solution of hydrochloric acid. In some embodiments, step d) is conducted by acidifying a filtrate of a reaction mixture of step c). In some embodiments, the step d) is conducted by treating an aqueous filtrate of step c) with an aqueous solution of hydrochloric acid to form a mixture with a pH value of from about 9 to about 10. In some embodiments, the step d) is conducted by i) treating an aqueous filtrate of step c) with a first aqueous solution of hydrochloric acid (e.g., 6M) to form a first slurry having a pH value of from about 9 to about 10; ii) further treating the first slurry with a second aqueous solution of hydrochloric acid (e.g., 2M) to form a second slurry having a pH value of from about 7 to about 8; and mixing the second slurry, wherein the first and/or second slurry includes Compound 1, a tautomer, a salt, a hydrate, or a combination thereof. In some embodiments, when step c) is conducted with sodium hydroxide in water; the second slurry includes Compound 1, substantially in a mono-sodium salt form represented by formula (Ib):

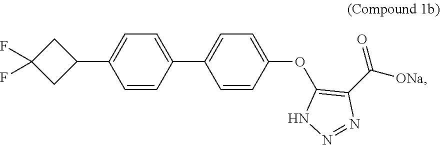

or a tautomer thereof.

In general, step d) can be performed at an ambient to an elevated temperature. For example, the reaction mixture of step d) can be at a temperature of from 20° C. to 50° C. In some embodiments, step d) is conducted at a temperature of from 20° C. to 35° C., or at room temperature. In some embodiments, step d) is conducted at room temperature. In some embodiments, step d) is conducted at a temperature of from 20° C. to 35° C.

In some embodiments, steps c) and d) are each conducted in an aqueous solution.

### Compound of Formula (III)

In some embodiments, the compound of formula (III) is prepared by: combining 1-bromo-4-(3,3-difluorocyclobutyl)benzene represented by the formula:

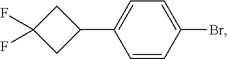 [SMILES: FC1(F)CC(c2ccc(Br)cc2)C1]

or a salt thereof, (4-hydroxyphenyl)boronic acid represented by the formula:

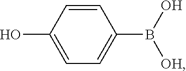

a palladium catalyst, and a third base in a fourth solvent to provide the compound of formula (III):

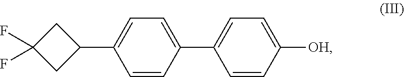 [SMILES: Oc1ccc(-c2ccc(C3CC(F)(F)C3)cc2)cc1]

or a salt thereof.

In some embodiments, the palladium catalyst is Pd(acac)₂, [Pd(allyl)Cl]₂, Pd(CH₃CN)₂Cl₂, Pd(dba)₂, Pd(CH₃COO)₂, Pd₂(dba)₃, Pd₂(dba)₃·CHCl₃, Pd(PPh₃)₄, Pd(OAc)₂, Pd(PCy₃)₂Cl₂, Pd(PPh₃)₂Cl₂, Pd[P(o-tol)₃]₂Cl₂, Pd(amphos)Cl₂, Pd(dppf)Cl₂, Pd(dppf)Cl₂·CH₂Cl₂, Pd(dtbpf)Cl₂, Pd(CH₃CN)₄(BF₄)₂, PdCl₂, XPhos-Pd-G3, Pd-PEPPSI™-IPr, Pd—PEPPSI™-SIPr, or Pd-PEPPSI™-IPent. In some embodiments, the palladium catalyst is [Pd(OAc)₂]₃, Na₂PdCl₄, Pd(CH₃CN)₄(CF₃SO₃)₂, bis(benzonitrile)palladium(II) dichloride, palladium(II)(π-cinnamyl) chloride dimer, [di-tert-butyl(chloro)-phosphine]-palladium(II) dichloride dimer, dihydrogen di-p-chlorotetrakis(di-tert-butylphosphinito)-dipalladate, di-p-chlorobis(2′-amino-1,1′-biphenyl-2-yl-C,N)-dipalladium(II), chloro[(tri-tertbutylphosphine)-2-(2-aminobiphenyl)]-palladium(II), Najera catalyst I, Najera catalyst II, di-p-chlorobis[2-[(dimethylamino)-methyl]-phenyl-C,N]dipalladium(II), di-p-chlorobis[2-[(dimethylamino)-methyl]-4,6-dimethoxyphenyl-C,N]-dipalladium(II), 1,2-bis(diphenylphosphino)-ethane]palladium(II) dichloride, [1,3-bis(diphenylphosphino)-propane]palladium(II) dichloride, [1,4-bis(diphenylphosphino)-butane]palladium(II) dichloride, bis(methyldiphenylphosphine) palladium(II) dichloride, benzylbis(triphenylphosphine)palladium(II) chloride, bis(triphenylphosphine)-palladium(II) diacetate, dichloro(1,5-cyclooctadiene)palladium(II), dichloro[9,9-dimethyl-4,5-bis(diphenylphosphino)xanthene]-palladium(II), SingaCycle™-A1, SingaCycle™-A2, SingaCycle™-A3, SingaCycle™-A4, bis(tri-tert-butylphosphine)palladium(0), bis[di-tert-butyl(4-dimethylaminophenyl)phosphine]-palladium(0), bis[1,2-bis(diphenylphosphino)ethane]-palladium(0), poly(methylphenyl)silane supported palladium/alumina hybrid catalyst, polydimethylsilane supported palladium/alumina hybrid catalyst, or poly[N-isopropylacrylamide-co-4-(diphenylphosphino)-styrene]palladium(II) dichloride (ratio, acrylamide:phosphine=20:2). In some embodiments, the palladium catalyst is Pd(dppf)Cl₂, Pd(dppf)Cl₂·CH₂Cl₂, or Pd(dtbpf)Cl₂. In some embodiments, the palladium catalyst is Pd(dppf)Cl₂·CH₂Cl₂.

In some embodiments, the palladium catalyst is in a substoichiometric amount. In some embodiments, Pd(dppf)Cl₂·CH₂Cl₂ is in a substoichiometric amount. In some embodiments, the palladium catalyst is in an amount of from about 0.001 to about 0.5 equivalent, from about 0.001 to about 0.1 equivalent, or from about 0.002 to about 0.01 equivalent, relative to 1-bromo-4-(3,3-difluorocyclobutyl)benzene. In some embodiments, the palladium catalyst is in an amount of from about 0.002 to about 0.01 equivalent relative to 1-bromo-4-(3,3-difluorocyclobutyl)benzene. In some embodiments, Pd(dppf)Cl₂·CH₂Cl₂ is in an amount of from about 0.002 to about 0.01 equivalent relative to 1-bromo-4-(3,3-difluorocyclobutyl)benzene.

The third base can be an alkali carbonate, an alkali bicarbonate, an alkali phosphate tribasic, an alkali hydroxide, an alkali carboxylate, an amine, an alkali alkoxide, or a combination thereof, each of which is described herein. In some embodiments, the third base is sodium carbonate, potassium carbonate, cesium carbonate, sodium phosphate tribasic, potassium phosphate tribasic, sodium acetate, potassium acetate, cesium acetate, or a combination thereof. In some embodiments, the third base is sodium carbonate, potassium carbonate, or cesium carbonate. In some embodiments, the third base includes sodium carbonate. In some embodiments, the third base is p sodium carbonate.

In some embodiments, the third base is in an amount of from about 1 to about 5 equivalents, from about 1 to about 4 equivalents, from about 1 to about 3 equivalents, from about 1 to about 2 equivalents, or about 1.0 equivalent, relative to (4-hydroxyphenyl)boronic acid. In some embodiments, the third base is in an amount of from about 1 to about 2 equivalents or about 1.0 equivalent, relative to (4-hydroxyphenyl)boronic acid. In some embodiments, the third base is in an amount of from about 1 to about 2 equivalents, relative to (4-hydroxyphenyl)boronic acid. In some embodiments, the third base is in an amount of about 1.0 equivalents, relative to (4-hydroxyphenyl)boronic acid.

In some embodiments, 1-bromo-4-(3,3-difluorocyclobutyl)benzene is in an amount of from about 1.0 to about 2.0 equivalents, from about 1.1 to about 2.0 equivalents, from about 1.1 to about 1.5 equivalents, or about 1.3 equivalents, relative to (4-hydroxyphenyl)boronic acid. In some embodiments, 1-bromo-4-(3,3-difluorocyclobutyl)benzene is in an amount of from about 1.1 to about 1.5 equivalents or about 1.3 equivalents, relative to (4-hydroxyphenyl)boronic acid. In some embodiments, 1-bromo-4-(3,3-difluorocyclobutyl)benzene is in an amount of from about 1.1 to about 1.5 equivalents, relative to (4-hydroxyphenyl)boronic acid. In some embodiments, 1-bromo-4-(3,3-difluorocyclobutyl)benzene is in an amount of about 1.3 equivalents, relative to (4-hydroxyphenyl)boronic acid.

The fourth solvent can be any suitable polar or non-polar, protic or aprotic solvent. In some embodiments, the fourth solvent is water, C₁₋₄ alcohol, benzene, toluene, dioxane, tetrahydrofuran (THF), 2-methyl-tetrahydrofuran (MeTHF), acetonitrile (ACN), N-methylpyrrolidone (NMP), N,N-dimethylformamide (DMF), N,N-dimethylacetamide (DMAC), dimethoxyethane (DME), ethylene glycol, or a combination thereof. In some embodiments, the fourth solvent includes dioxane. In some embodiments, the fourth solvent includes dioxane and water. In some embodiments, the fourth solvent includes 1,4-dioxane. In some embodiments, the fourth solvent includes 1,4-dioxane and water. In some embodiments, the fourth solvent is a mixture of 1,4-dioxane and water.

In general, the Suzuki reaction to form the compound of formula (III) can be performed at an ambient to an elevated temperature. For example, the reaction mixture can be at a temperature of from 30° C. to 110° C. or heated to reflux. In some embodiments, the reaction is conducted at a temperature of from about 30° C. to about 100° C., from about 40° C. to about 80° C., from about 40° C. to about 60° C., or from about 40° C. to about 50° C. In some embodiments, the reaction is conducted at a temperature of from about 40° C. to about 50° C.

In another aspect, the present disclosure provides a compound, represented by formula (III):

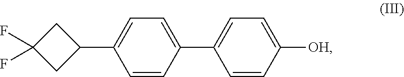 [SMILES: Oc1ccc(-c2ccc(C3CC(F)(F)C3)cc2)cc1]

or a salt thereof.

### Compound 1 Via Steps a) to d)

In another aspect, the present disclosure provides a process for preparing Compound 1 represented by the formula:

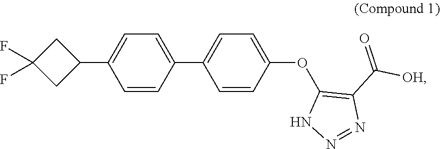 [SMILES: O=C(O)c1nn[nH]c1Oc1ccc(-c2ccc(C3CC(F)(F)C3)cc2)cc1]

a tautomer, a sodium salt, a hydrate, or a combination thereof, via steps a) to d). The process includes:
a) combining a compound of formula (IVa):

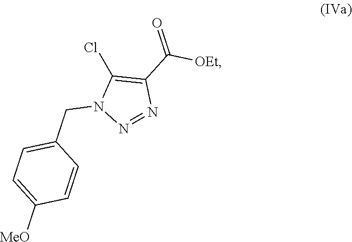 [SMILES: CCOC(=O)c1nnn(Cc2ccc(OC)cc2)c1Cl]

or a salt thereof, a compound of formula (III):

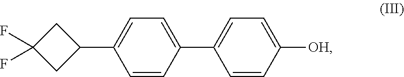 [SMILES: Oc1ccc(-c2ccc(C3CC(F)(F)C3)cc2)cc1]

or a salt thereof, and a first base in a first solvent to form a compound of formula (IIa):

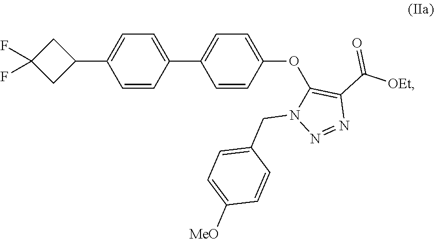 [SMILES: CCOC(=O)c1nnn(Cc2ccc(OC)cc2)c1Oc1ccc(-c2ccc(C3CC(F)(F)C3)cc2)cc1]

or a salt thereof, wherein the first base is 1,8-diazabicyclo[5.4.0]undec-7-ene (DBU) and the first solvent includes N,N-dimethylacetamide (DMAC); or the first base is cesium carbonate and the first solvent includes N-methylpyrrolidone (NMP);
b) treating the compound of formula (IIa) or the salt thereof, with trifluoroacetic acid and anisole to provide a compound of formula (Ia-1):

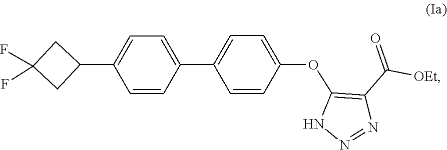 [SMILES: CCOC(=O)c1nn[nH]c1Oc1ccc(-c2ccc(C3CC(F)(F)C3)cc2)cc1]

or a tautomer and/or a salt thereof;
c) saponifying the compound of formula (Ia), or the tautomer and/or the salt thereof, with an aqueous solution of sodium hydroxide; and
d) acidifying with an aqueous solution of HCl to provide Compound 1, the tautomer, the sodium salt, the hydrate, or the combination thereof.

In some embodiments, steps a) to d) are conducted according to any of embodiments that are applicable, as described above.

With reference to step a), in some embodiments, he first base is 1,8-diazabicyclo[5.4.0]undec-7-ene (DBU) and the first solvent includes N,N-dimethylacetamide (DMAC). In some embodiments, first base is 1,8-diazabicyclo[5.4.0]undec-7-ene (DBU) and the first solvent is N,N-dimethylacetamide (DMAC). In some embodiments, the compound of formula (III) is in an amount of from about 1.0 to about 1.2 equivalents and DBU is in an amount of from about 1.2 to about 1.5 equivalents, relative to the compound of formula (IVa). In some embodiments, the compound of formula (III) is in an amount of about 1.0 equivalent and DBU is in an amount of about 1.35 equivalents, relative to the compound of formula (IVa). In some embodiments, step a) is conducted at a temperature of from about 85° C. to about 90° C.

In some embodiments of step a), the first base is cesium carbonate and the first solvent includes N-methylpyrrolidone (NMP). In some embodiments of step a), the first base is cesium carbonate and the first solvent is N-methylpyrrolidone (NMP).

With reference to step b), in some embodiments, trifluoroacetic acid is in an access amount, relative to the compound of formula (IIa). In some embodiments, anisole is in an amount of from about 2 to about 5 equivalents and trifluoroacetic acid is in an amount of from about 10 to about 30 equivalents, relative to the compound of formula (IIa). In some embodiments, anisole is in an amount of about 3 equivalents and trifluoroacetic acid is in an amount of about 15 equivalents, relative to the compound of formula (IIa). In some embodiments, step b) is conducted at a temperature of from about 50° C. to about 60° C.

With reference to step c), in some embodiments, sodium hydroxide is in an amount of from about 2 to about 5 equivalents relative to the compound of formula (Ia). In some embodiments, sodium hydroxide is in an amount of about 3 equivalents relative to the compound of formula (Ia). In some embodiments, step c) is conducted at a temperature of from about 75° C. to about 85° C.

In some embodiments, step d) is conducted by acidifying a filtrate of a reaction mixture of step c).

In some embodiments, step d) is conducted according to steps:
d1) adding an aqueous 6M HCl solution to form a first slurry having a pH value of from about 9 to about 10;
d1) adding an aqueous 2M HCl solution to the first slurry to form a second slurry having a pH value of from about 7 to about 8;
d3) mixing the second slurry; and
d4) isolating a solid from the second slurry to provide Compound 1, substantially in a mono-sodium salt form represented by formula (Ib):

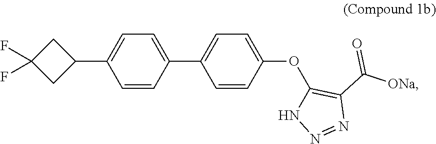

or a tautomer thereof.

In general, the solid from the second slurry can be isolated by convention methods (e.g., filtration, decanting, centrifuging). In some embodiments, isolating includes filtering the solid from the second slurry. The filtered solids can be further processed, for example washed with water for several times followed wash with an organic solvent (e.g., n-heptane), and then dried (e.g., under vacuum at a suitable temperature). In some embodiments, the solid first isolated from step d) is washed with water followed by wash with n-heptane; and is dried under vacuum at a temperature of from about 30° C. to about 40° C. to provide Compound 1, substantially in a mono-sodium salt form of formula (Ib).

### VIII. Examples

### Instruments and Methods

X-ray Powder Diffraction (XRPD): X-ray powder diffraction data were collected under ambient conditions on a Bruker D2 PHASER diffractometer with a low power X-ray generator of 300 W. Powder patterns were collected on a zero background sample holder with a 0.15 s/step with a total step of 1837, two theta at 0.02° per step at 30 kV and 10 mA. The X-ray tube of Cu (Kα) was employed, with the Kα2/Kα1 intensity ratio of 0.50 (1.54439 Å/1.5406 Å).

Thermogravimetric Analysis (TGA): Thermogravimetric analysis data were collected with a TA Discovery series TGA. A few milligrams of material was heated from room temperature to 350° C. with a heating rate of 10° C. per minute under nitrogen protection.

Differential Scanning Calorimetry (DSC): Differential scanning calorimetry was performed with a TA Discovery series DSC using approximately a few milligrams of material in a Tzero aluminum pan sealed with a Tzero hermetic lid. Samples were analyzed using a heating rate of 10° C. per minute under 50 mL per minute of nitrogen flow.

Polarized Light Microscopy (PLM): Photomicrographs were taken using an Olympus BX53M polarized light microscope at room temperature (rt).

High Performance Liquid Chromatography (HPLC): HPLC of Agilent 1260 with DAD detector was employed to detect the purity and solubility, with the methods shown in Table 1.

TABLE 1
HPLC method for purity and solubility tests

| Instrument | Agilent 1260 HPLC with DAD detector | |
|---|---|---|
| Column | Agilent Poroshell EC-C18 | |
| | (4.6 mm × 100 mm, 2.7 μm) | |
| Mobile phase | A: 0.025% TFA in water, | |
| | B: 0.025% TFA in ACN | |
| | Time (min) | % B |
| Gradient | 0.0 | 10 |
| | 0.1 | 10 |
| | 11.0 | 90 |
| | 18.0 | 90 |
| | 18.1 | 10 |
| | 20.0 | 10 |

| | |
|---|---|
| Detector | UV, 220 nm |
| Column temperature | 25° C. |
| Flow rate | 1.0 mL/min |
| Injection volume | 1 μL |
| Diluent | ACN:H₂O = 80:20 (v/v) |

Dynamic Vapor Sorption (DVS): Dynamic vapor sorption was performed with an ADVENTURE series DVS at 25° C. under nitrogen blow. Approximately 20 milligrams of material was used. Samples were analyzed using method below:
1. Ambient relative humidity to 95% RH at 10% RH (5% from 90% RH to 95% RH)
2. 95% RH to 0% RH at 10% RH (5% RH from 95% RH to 90% RH)
3. 0% RH to 95% RH at 10% RH (5% RH from 90% RH to 95% RH)

Inductively Coupled Plasma-Optical Emission Spectroscopy (ICP-OES): ICP-OES Avio 200 (Perkin-Elmer) was employed to determine the content of sodium salts, with the methods shown in Table 2.

TABLE 2
ICP-OES method for sodium content determination

| | |
|---|---|
| Instrument | ICP-OES Avio 200 (Perkin-Elmer) |
| RF-Power | 1400 W |
| Nebulizer flow | 0.7 L/min |
| Plasma flow | N/A |
| Auxiliary flow | 0.6 L/min |
| Injection mode | Organic injection |
| Processing mode | Peak area, external standard |
| Emission | Zn 589.592 nm |
| wavelength | |
| Sample flow rate | 1.2 mL/min |
| Plasma view | Axial |
| Standard | 100 mg/L of Na standard solution |
| Diluent solution | 60% NMP in H₂O(v/v) |
| Sample solution | Weight 100 mg of sample into a 10 mL volumetric flask, and dilute to volume |
| | with diluent. Prepare in duplicate. |
| 1# Calibration | Prepare 0.01 mg/L, 0.1 mg/L, 0.5 mg/L and 1.0 mg/L calibration solution, which |
| solutions | contain Zn. |
| Evaluation | The contents of contain Na are determined by external method, which can be |
| | directly calculated by workstation. |
| Injection order | Example: Blank, 1# Calibration solutions, Blank, Sample solution |

Solvent Abbreviation: The abbreviations of solvents used were listed in Table 3.

TABLE 3
Solvent abbreviation list

| Abbreviation | Solvent | Abbreviation | Solvent |
|---|---|---|---|
| MeOH | Methanol | EtOAc | Ethyl acetate |
| EtOH | Ethanol | ACN | Acetonitrile |
| IPA | Isopropanol | DCM | Dichloromethane |
| THF | Tetrahydrofuran | H₂O | Water |
| MTBE | Methyl tert- | DMAc | N,N- |
| | butyl ether | | Dimethylacetamide |

### Example 1: Preparation of Crystalline Form a of Compound 1a

Crystalline Form A was prepared on a 230-mg scale according to the procedures as described below.
1. Weigh 500.6 mg starting material (including Compounds 1, 1a, and 1b) to a 20-mL vial;
2. Add 15 mL THF/H₂O (1:3, v/v) to the vial. (23° C., 70% RH);
3. Stir at 50° C. at 800 rpm for ~3 hrs to get a suspension;
4. Filter the suspension to a clean 20-mL vial and add 1~2 mg Form A seeds to the filtrates;
5. Transfer the sample to cool at 5° C.;
6. After 2.5 days, isolate the solids by vacuum filtration, check the wet cake by XRPD and it conformed to Form A;
7. Vacuum dry the solids at room temperature (rt) for 3 hours; and
8. Collect 227.8 mg of solids.

The solids isolated has a chemical purity of 99.7 area % by a HPLC method as described herein.

### Example 2: Crystalline Form A of Compound 1a

Crystalline Form A of Compound 1a, prepared using similar procedures of Example 1 on a small scale, was used for analyses of XRPD, DSC, TGA, PLM, ICP-OES, and post-heating XRPD. Crystalline Form A of Compound 1a from Example 1 was used for the DVS analysis, solubility studies, and other confirmatory analyses (e.g., XRPD, TGA, DSC, and ICP-OES). Form A was characterized by methods as shown in Table 4.

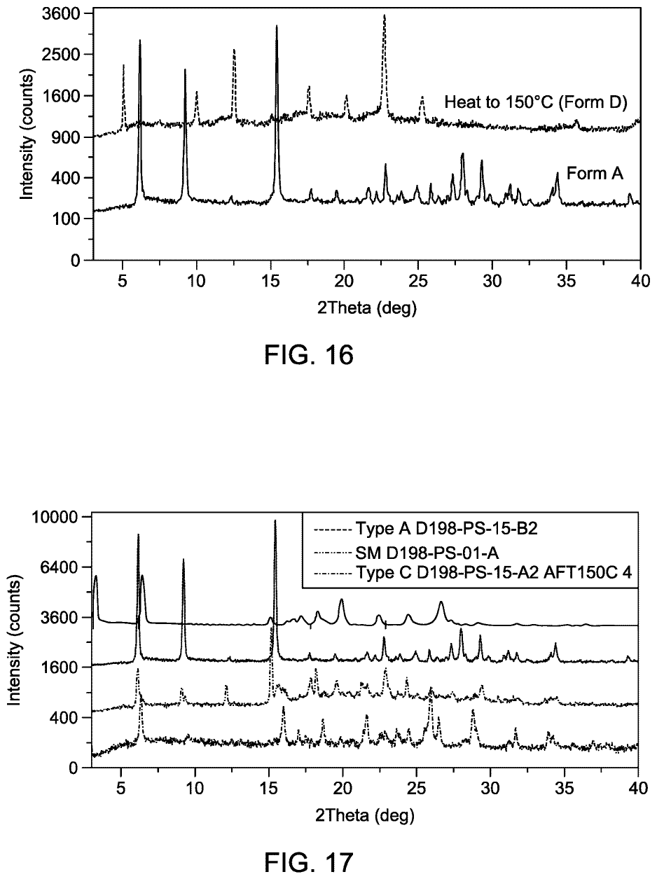

TABLE 4
Characterization of Crystalline Form A

| Method | Result | FIG. |
|---|---|---|
| XRPD | Form A; high crystallinity | FIG. 1 |
| DSC | a broad endothermic peak | FIG. 2 |
| | at 118.5° C. (with an | |
| | onset of 89.6° C.); and | |
| | two sharp endothermic peaks | |
| | at 240.8 and 243.4° C. | |
| TGA | a weight loss of 4.2% up to 150° C. | FIG. 3 |
| PLM | flake birefringent crystals | FIG. 4 |
| DVS | 10-95% RH: <0.1% wt gain; and | FIG. 5 |
| | 95-10% RH: <0.1% wt loss | |
| ICP-OES | Sodium content: 5.4 wt % | — |
| post-heating XRPD | Form D | FIG. 16 |

Form A showed high crystallinity by an X-ray powder diffraction (XRPD) in FIG. 1 (with observed peaks listed in Table 5) and flake birefringent crystals by a polarized light microscopy (PLM) of FIG. 4. As shown by TGA and DSC data in FIG. 2 and FIG. 3, the sample exhibited a weight loss of 4.2% up to 150° C. by TGA, and DSC result showed a broad endotherm at 89.6° C. (onset) due to dehydration/desolvation followed by two sharp endothermic peaks at 240.8° C. and 243.4° C.

TABLE 5
Observed peaks of XRPD pattern as shown in FIG. 1 of Form A

| degrees 2θ (±0.2 | d space | Relative |
|---|---|---|
| degrees 2θ) | (Å) | Intensity |
| 6.2 | 14.36949 | 82.2% |
| 9.2 | 9.59333 | 64.7% |
| 12.3 | 7.19253 | 0.8% |
| 15.4 | 5.74488 | 100.0% |
| 17.7 | 4.99894 | 3.4% |
| 19.5 | 4.55747 | 2.9% |
| 20.9 | 4.25693 | 1.0% |
| 21.6 | 4.10493 | 3.9% |
| 22.2 | 4.00696 | 3.0% |
| 22.8 | 3.90281 | 12.0% |
| 23.6 | 3.76957 | 2.1% |
| 23.8 | 3.72879 | 3.4% |
| 24.9 | 3.57205 | 5.0% |
| 25.8 | 3.44575 | 4.8% |
| 26.4 | 3.37874 | 1.3% |
| 27.0 | 3.30418 | 1.7% |
| 27.3 | 3.26204 | 8.6% |
| 28.0 | 3.18614 | 16.2% |
| 28.3 | 3.15446 | 3.4% |
| 29.0 | 3.07751 | 1.9% |
| 29.3 | 3.04756 | 14.4% |
| 29.8 | 2.99316 | 2.9% |
| 30.9 | 2.89271 | 3.1% |
| 31.2 | 2.86666 | 4.8% |
| 31.7 | 2.81683 | 4.8% |
| 34.1 | 2.63011 | 4.6% |
| 34.4 | 2.60757 | 9.9% |
| 38.2 | 2.35608 | 0.8% |
| 39.3 | 2.29271 | 3.4% |

Heating experiments were further performed to understand the thermal events. Form A sample was heated to desired temperatures and then cooled down to room temperature (rt) under N₂ blow for XRPD analysis. As shown in FIG. 16, a new crystal form named as Form D was obtained after heating to 150° C., indicating Form A was a hydrate or solvate, and yellow sample was observed after heating to 242° C., suggestive of decomposition. Considering that Form A has a weight loss of 4.2% by TGA, it was likely in a mono-hydrate form. The sodium (Na) content of the sample was further determined to be 5.4 wt % by ICP-OES, with a molar ratio of 1.02 (Na/Compound 1). All the data indicated that Form A was likely in a mono-sodium salt, monohydrate form.

### Hygroscopicity

To understand the hygroscopicity of Form A, dynamic vapor sorption (DVS) was employed to measure the mass change as a function of relative humidity at 25° C. Form A was equilibrated at ambient relative humidity before analysis.

As the results shown in FIG. 5, Form A showed negligible water uptake at 25° C./80% RH as compared to the starting point (60% RH), indicating the sample was non-hygroscopic. Also the sample lost ~0.6% water rapidly when the relative humidity was below 10% RH, suggesting the partial dehydration. The sample after DVS test was equilibrated at 0% RH overnight, and a few diffractive peaks of anhydrate Form D was observed. The results indicated good form stability for Form A under a broad humidity range.

### Solubility

Solubility at pH 2-10: pH solubility measurement was conducted in six pH buffers (pH 2.0/4.0/6.0/7.0/8.0/10.0) at 25° C. for 24 hrs to understand the solubility and form stability of Form A. Specifically, about 5~10 mg Form A sample was added to 1.0 mL corresponding pH buffer. After shaking at 100 rpm at 25° C. for 24 hrs, the suspensions were centrifuged. The solids were analyzed by XRPD for crystal form, and the filtrates of supernatants were analyzed by HPLC for solubility and by pH meter for pH value.

The results are summarized in Table 6. The sample showed increasing solubility (0~4.8 mg/mL) as a function of pH and limited solubility was detected for samples in pH 2.0/4.0/6.0. In addition, samples in pH 2.0~7.0 showed new diffractive peaks, while no form change was observed for samples in pH 8.0/10.0 conditions.

TABLE 6
24-hr Solubility Profile of Form A at pH 2-10, 25° C.

| pH Buffer | Solubility | Final pH | Form |
|---|---|---|---|
| Condition | (mg/mL) | (measured) | Change |
| pH 2.0 | <LOD | pH 2.5 | Yes |
| pH 4.0 | <LOD | pH 4.4 | Yes |
| pH 6.0 | <LOD | pH 6.2 | Yes |
| pH 7.0 | 0.055 | pH 7.1 | Yes |
| pH 8.0 | 0.404 | pH 7.4 | No |
| pH 10.0 | 4.826 | pH 8.4 | No |

LOD: Limit of Detection

Solubility in Water at various Temperature: Solubility test was conducted for Form A in water at various temperatures. Specifically, about 3~5 mg Form A sample was added to 1 mL water. After stirring at 750 rpm at desired temperatures (25/35/50/60/70° C.) for 1~2 hrs, solids were isolated for XRPD to check the crystal form and the supernatants were collected for a HPLC analysis to detect the solubility.

The results are summarized in Table 7. The sample showed increasing solubility as a function of temperature (1.1~2.3 mg/mL), while partial conversion to Form C was observed, indicating form transformation has occurred.

TABLE 7
Solubility Profile of Form A in Water at Various Temperatures

| Temperature | Solubility | Final |
|---|---|---|
| (° C.) | (mg/mL) | Form |
| 25° C. | 1.10 | Forms A + C |
| 35° C. | 1.30 | ND |
| 50° C. | 1.94 | ND |
| 60° C. | 1.86 | Forms A + C |
| 70° C. | 2.32 | ND |

Solubility in Various Solvents: Solubility of Form A was also measured in 20% EtOH in water and 20% DMAc in water at room temperature (rt). Specifically, about 3 mg Form A sample was added to 0.6 mL solvent. After stirring at 750 rpm at rt (−25° C.) for 20~60 mins, the suspension was filtered for solubility test by HPLC. The solubility was found to be 1.9 mg/mL in 20% EtOH in water and 3.3 mg/mL in 20% DMAc in water.

### Summary

Crystalline Form A of Compound 1a (mono-sodium, monohydrate) was evaluated and found to have properties as follows: 1) Form A showed non-hygroscopicity, and good form stability under different humidity; 2) The sample showed increasing solubility (0~4.8 mg/mL) as a function of pH (2.0~10.0) at 25° C. and increasing solubility (1.1~2.3 mg/mL) as a function of temperature (25~70° C.); 3) the solubility of Form A sample was found to be 1.9 mg/mL in 20% EtOH in water and 3.3 mg/mL in 20% DMAc in water at room temperature.

### Example 3: Crystalline Form D of Compound 1b

Form D was generated via dehydration of monohydrate Form A. Accordingly, Form D was obtained via heating Form A to 150° C. followed by cooling to room temperature under N₂ blow. Form A was characterized by methods as shown in Table 8.

TABLE 8
Characterization of Crystalline Form D

| Method | Result | FIG. |
|---|---|---|
| XRPD | Form D | FIG. 6 |
| DSC | two overlapped endothermic | FIG. 7 |
| | peaks at 241.2° C. and | |
| | 243.2° C. | |
| TGA | a weight loss of 0.6% | FIG. 8 |
| | up to 150° C. | |
| PLM | crystals | FIG. 9 |

TABLE 9
Observed peaks of XRPD pattern as shown in FIG. 6 of Form D

| degrees 2θ (±0.2 | d space | Relative |
|---|---|---|
| degrees 2θ) | (Å) | Intensity |
| 5.0 | 17.51875 | 50.9% |
| 5.9 | 14.92109 | 4.3% |
| 6.4 | 13.83863 | 5.2% |
| 6.8 | 12.95150 | 3.2% |
| 7.5 | 11.81559 | 7.6% |
| 10.0 | 8.84457 | 25.8% |
| 12.5 | 7.06261 | 68.0% |
| 15.1 | 5.87836 | 9.6% |
| 17.6 | 5.03820 | 24.3% |
| 18.3 | 4.83939 | 6.0% |
| 20.1 | 4.40579 | 18.6% |
| 22.3 | 3.98652 | 6.4% |
| 22.7 | 3.91532 | 100.0% |
| 24.5 | 3.63282 | 3.3% |
| 25.3 | 3.52230 | 20.4% |
| 26.2 | 3.40493 | 4.3% |
| 28.5 | 3.12957 | 3.4% |
| 33.5 | 2.66995 | 4.6% |
| 35.7 | 2.51305 | 5.8% |
| 37.3 | 2.40820 | 4.6% |

As shown by TGA and DSC data, the sample exhibited a weight loss of 0.6% before 150° C. and two overlapped endotherms at 241.2 and 243.2° C. (peak) corresponding to melting and decomposition. The data indicated that Form D is an anhydrate.

### Example 4: Crystalline Form E of Compound 1b

Form E was produced via slurry of mono-sodium salt Form A and Form D in THF/H₂O (a_{w} of 0~0.8) at room temperature. Accordingly, Form E was obtained via slurry the mixture of Forms A and D in THF at room temperature for characterization. Form D was characterized by methods as shown in Table 10.

TABLE 10
Characterization of Crystalline Form E

| Method | Result | FIG. |
|---|---|---|
| XRPD | Form E; low crystallinity | FIG. 10 |
| DSC | a broad endothermic peak at 64.5; and | FIG. 11 |
| | a sharp endothermic peak at 239.3° C. | |
| TGA | a weight loss of 2.1% up to 150° C. | FIG. 12 |
| post-heating/ | Form E (no form change); | — |
| cooling XRPD | lower crystallinity | |

TABLE 11
Observed peaks of XRPD pattern as shown in FIG. 10 of Form E

| degrees 2θ (±0.2 | d space | Relative |
|---|---|---|
| degrees 2θ) | (Å) | Intensity |
| 5.3 | 16.59328 | 22.7% |
| 12.7 | 6.98961 | 12.5% |
| 16.0 | 5.55371 | 100.0% |
| 19.6 | 4.53801 | 40.3% |
| 23.0 | 3.86949 | 38.1% |
| 25.1 | 3.54410 | 33.2% |
| 28.9 | 3.08781 | 25.8% |

Form E showed low crystallinity and no form change after dried at RT overnight by XRPD. As shown by TGA and DSC data, a weight loss of 2.1% was observed before 150° C. and DSC result showed a broad endotherm at 64.5° C. (peak) followed by melting at 232.5° C. (onset). After heating to 150° C. and then cooling down to room temperature under N₂ blow, the sample showed no form change while lower crystallinity was observed. Combined with the preparation conditions, the first endotherm in DSC was likely caused by dehydration. Form E is likely a hydrate (e.g., hemihydrate calculated based on the weight loss of 2.1% by TGA).

### Example 5: Co-crystalline Form C of Compounds 1 and 1b

Form C was generated via slow cooling in MeOH or anti-solvent addition in (THF/H₂O)/H₂O and (ACN/H₂O)/H₂O in screening. Accordingly, Form C was obtained via anti-solvent addition at room temperature using THF/H₂O (1:1, v/v) as good solvent and H₂O as anti-solvent. Form C was found to have a chemical purity of99.8% by a HPLC method. Form C was characterized by methods as shown in Table 12.

TABLE 12
Characterization of Crystalline Form C

| Method | Result | FIG. |
|---|---|---|
| XRPD | Form C; crystallinity | FIG. 13 |
| DSC | two overlapped endothermic | FIG. 14 |
| | peaks at 196.9° C. | |
| | and 215.1° C. | |
| TGA | a weight loss of 1.3% up to 150° C. | FIG. 15 |
| ICP-OES | Sodium content: 2.5 wt % | — |

TABLE 13
Observed peaks of XRPD pattern as shown in FIG. 13 of Form C

| degrees 2θ (±0.2 | d space | Relative |
|---|---|---|
| degrees 2θ) | (Å) | Intensity |
| 6.4 | 13.91187 | 48.5% |
| 9.2 | 9.64150 | 2.9% |
| 9.6 | 9.23572 | 7.6% |
| 15.4 | 5.75641 | 2.5% |
| 16.0 | 5.54017 | 39.6% |
| 17.0 | 5.21545 | 15.8% |
| 17.5 | 5.06355 | 6.4% |
| 18.7 | 4.75017 | 34.0% |
| 19.8 | 4.47060 | 13.2% |
| 20.3 | 4.38125 | 10.4% |
| 21.6 | 4.11154 | 50.6% |
| 22.5 | 3.94163 | 5.5% |
| 22.9 | 3.88664 | 10.0% |
| 23.6 | 3.76893 | 16.0% |
| 24.4 | 3.64046 | 20.3% |
| 25.9 | 3.43560 | 100.0% |
| 26.5 | 3.36347 | 34.8% |
| 28.8 | 3.09914 | 56.8% |
| 31.7 | 2.82317 | 16.3% |
| 33.9 | 2.64175 | 11.0% |
| 34.1 | 2.62476 | 10.2% |
| 35.6 | 2.52071 | 3.2% |
| 37.0 | 2.43057 | 4.1% |
| 37.5 | 2.39900 | 5.0% |
| 37.9 | 2.37072 | 2.7% |

As shown by TGA and DSC data, Form C exhibited a weight loss of 1.3% before 150° C. and two overlapped endotherms at 196.9 and 215.1° C. (peak) possibly due to melting accompanied with decomposition, indicating that Form C was an anhydrate. Form C was found to have Na content to be 2.5 wt % by ICP-OES, with a molar ratio of 0.41 (Na/Compound 1). All the data indicated Form C is an anhydrate, likely co-crystal of mono-sodium salt of Compound 1b and an acid of Compound 1.

### Example 6: Hydrate and Polymorph Screening

### A. Starting Material Characterization

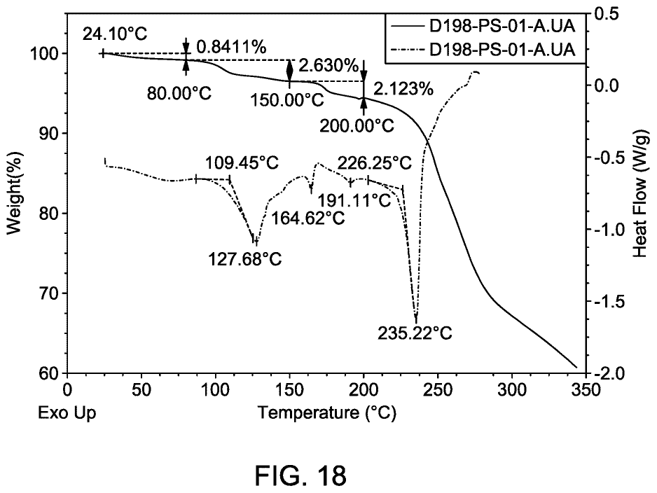

The starting material for screening was characterized by XRPD, TGA, DSC, and ICP-OES. The XRPD results (FIG. 17) indicated that it was crystalline with apparent amorphous halo; and the crystal form was identified to be a mixture of mono-sodium salt, monohydrate Form A, co-crystalline (an acid and mono-sodium salt) anhydrate Form C and an acid (represented by the formula of Compound 1). As per TGA and DSC data (FIG. 18), a three-step weight loss of 5.6% was observed before 200° C., and DSC result showed four endothermic peaks at 127.7° C., 164.6° C., 191.1° C., and 235.2° C. before decomposition. The Na content of the sample was determined to be 7.3 wt % by ICP-OES.

The solubility of starting material was estimated at room temperature. Specifically, approximate 2 mg solids were added into a HPLC glass vial. Solvents in Table 14 were then added stepwise (50/50/200/700 μL) into the vials until the solids were dissolved or a total volume of 1.0 mL was reached. The solubility data were used to guide the solvent selection in hydrate and polymorph screening.

TABLE 14
Approximate Solubility of Starting Material at Room Temperature

| Solvent | Solubility (mg/mL) | Solvent (v:v) | Solubility (mg/mL) |
|---|---|---|---|
| DMAc | 4.2 < S < 7.0 | n-Heptane | S < 2.0 |
| MeOH | 2.2 < S < 7.3 | H₂O | S < 2.6 |
| EtOH | S < 2.2 | MeOH/DCM (1:1) | S < 2.4 |
| IPA | S < 2.1 | IPA/ACN (3:2) | S < 2.1 |
| Acetone | S < 2.1 | THF/H₂O (1:1) | 22.0 < S < 44.0 |
| EtOAc | S < 2.1 | THF/H₂O (5:1) | 8.3 < S < 25.0 |
| MTBE | S < 2.2 | THF/H₂O (1:5) | S < 2.4 |
| THF | S < 2.2 | EtOH/H₂O (1:1) | S < 2.2 |
| Toluene | S < 2.2 | EtOH/H₂O (1:5) | S < 2.3 |
| ACN | S < 2.5 | EtOH/H₂O (5:1) | S < 2.5 |
| DCM | S < 2.4 | ACN/H₂O (1:1) | 7.3 < S < 22.0 |

### B. Summary of Hydrate and Polymorph Screening

Hydrate screening was performed under 20 conditions in solvent systems containing water using the starting material (as characterized above), through methods of slurry at rt/50° C., anti-solvent addition and slow cooling. The details can be referred to Part C.

Polymorph screen was conducted under 45 conditions in process relevant solvents using the starting material (as characterized above), through methods of slurry at rt/50° C., slow cooling, solid vapor diffusion, anti-solvent addition, and polymer-induced crystallization. The details can be referred to Part D.

Hydrate and polymorph screening methods and the results are summarized in Table 15 and Table 16. Based on the characterization results, a total of five crystal forms were obtained from the screen and further investigation, including three mono-sodium salt crystal forms (monohydrate Form A, anhydrate Form D, and low crystallinity hemihydrate Form E), one likelihood co-crystalline Form C (mono-sodium salt and an acid, molar ratio of 1:1), and one metastable Form B. The characterization data are provided in Table 17.

TABLE 15
Hydrate Screening Summary of Compound 1, mono-sodium salt

| | No. | |
|---|---|---|
| Method | of Exp. | Crystal Form |
| Slurry at room temperature | 10 | Form A, Forms A + C |
| Slurry at 50° C. | 3 | Form A, Forms A + C |
| Anti-solvent addition | 3 | Form C |
| Slow cooling | 4 | Form A, Forms A + C |
| Total | 20 | Form A, Form C, Forms A + C |

TABLE 16
Polymorph Screening Summary of Compound 1, mono-sodium salt

| | No. of | |
|---|---|---|
| Method | Exp. | Crystal Form |
| Slurry at room temperature | 12 | Forms A/B/A + C/Low |
| | | crystallinity/Amorphous |
| Slurry at 50° C. | 7 | Forms A/A + C/Low crystallinity/Amorphous |
| Slow cooling | 2 | Form C |
| Solid vapor diffusion | 8 | Form A/Starting material/Low crystallinity |
| Anti-solvent addition | 12 | Form A/Amorphous |
| Polymer-induced | 4 | Form A, Forms A + C |
| crystallization | | |
| Total | 45 | Forms A/B/C/Low crystallinity/Amorphous |

TABLE 17
Characterization Results of Compound 1, Sodium Salt Crystalline Forms

| | | Wt. Loss in | Endo. in | | |
|---|---|---|---|---|---|
| | Preparation | TGA (%) | DSC | Molar Ratio | |
| Form | Condition | [rt to 150° C.] | (onset, ° C.) | (Na/API) | Form Identity |
| Form A | Quench cooling in | 4.2 | 118.5, 240.8, | 1.02 | Monohydrate, |
| | THF/H₂O (1:3, | | 243.4 | (5.4 wt % Na) | mono-sodium |
| | v/v) from 50° C. to | | | | salt |
| | rt | | | | |
| Form D | Heat Form A to | 0.5 | 241.2, 243.2 | — | Anhydrate, |
| | 150° C. | | | | mono-sodium |
| | | | | | salt |
| Form E | Slurry of Forms A + | 2.1 | 64.5, 239.3 | — | Hemihydrate, |
| | D in THF at rt | | | | mono-sodium |
| | | | | | salt |
| | | | | | Low crystallinity |
| Form C | Anti-solvent | 1.3 | 196.9, 215.1 | 0.41 | Anhydrate, co- |
| | addition at rt in | | | | crystal of mono- |
| | [(THF/H₂O)/H₂O] | | | | sodium salt with |
| | | | | | an acid of |
| | | | | | Compound 1 |
| Form B | Slurry of SM in | ND | ND | ND | Metastable form |
| | MeOH at rt | | | | (convert to |
| | | | | | Forms A + C at rt) |

room temperature (rt): 20~25° C., 40 to 90% RH; and ND: no data was collected since form transformation occurred upon drying at rt.

### C. Hydrate Screening Experiments

Slurry at room temperature: Slurry experiments were conducted at rt in various solvent systems. About 15 mg of starting material was suspended in 0.3 mL of solvents in HPLC vial. After the suspension was stirred magnetically for 2-7 days at rt, the remaining solids were isolated for XRPD analysis. Results summarized in Table 18 indicated that Form A of low crystallinity and a mixture of Forms A and C were obtained.

TABLE 18
Summary of slurry experiments at room temperature

| Experiment ID | Solvent (v:v) | Solid Form |
|---|---|---|
| C1-A1 | H₂O | Forms A + C |
| C1-A2 | EtOH/H₂O (a_{w} = 0.2, 970:30) | Form A (low) |
| C1-A3 | EtOH/H₂O (a_{w} = 0.4, 927:73) | Form A (low) |
| C1-A4 | EtOH/H₂O (a_{w} = 0.6, 855:145) | Form A (low) |
| C1-A5 | EtOH/H₂O (a_{w} = 0.8, 704:296) | Form A (low) |
| C1-A6 | IPA/H₂O (1:1) | Forms A + C |
| C1-A7 | THF/H₂O (1:2) | Form A (low) |
| C1-A8 | Acetone/H₂O (1:1) | Form A (low) |
| C1-A9 | ACN/H₂O (1:2) | Form A (low) |

Form A (low): difficult to judge whether they had Form C due to low crystallinity

Slurry at 50° C.: Slurry experiments were also conducted at 50° C. in various solvent systems. About 20 mg of starting material was suspended in 0.3 mL of solvent in HPLC vial. After the suspension was stirred for about 3 days at 50° C., the remaining solids were isolated for XRPD analysis. Results in Table 19 indicated that Form A of low crystallinity and a mixture of Forms A and C were generated.

TABLE 19
Summary of slurry experiments at 50° C.

| Experiment ID | Solvent (v:v) | Solid Form |
|---|---|---|
| C2-A1 | H₂O | Forms A + C |
| C2-A2 | EtOH/H₂O (1:1) | Form A (low) |
| C2-A3 | THF/H₂O (1:3) | Forms A + C |

Form A (low): difficult to judge whether they had Form C due to low crystallinity

Anti-solvent Addition: A total of three anti-solvent addition experiments were carried out at rt. About 6~15 mg of starting material was dissolved in 0.8~2.0 mL solvents to obtain a clear solution. The solution was magnetically stirred followed by addition of 0.2~0.5 mL anti-solvent stepwise till precipitate appeared or the total amount of anti-solvent reached 15.0 mL. The obtained precipitate was isolated for XRPD analysis. Results in Table 20 showed that Form C was obtained.

TABLE 20
Summary of anti-solvent addition experiments

| Experiment ID | Solvent (v:v) | Anti-solvent | Solid Form |
|---|---|---|---|
| C3-A1 | MeOH | H₂O | Clear |
| C3-A2 | THF/H₂O (1:1) | | Form C |
| C3-A3 | ACN/H₂O (1:1) | | Form C |

Clear solution was observed after evaporation at rt for more than 2 weeks

Slow Cooling: Slow cooling experiments were conducted in four solvent systems. About 20 mg of starting material was suspended in 1.5 mL of solvents in a 4-mL glass vial. The suspension was then heated to 50° C., equilibrated for 3 hrs and filtered using a Nylon membrane (pore size of 0.22 μm). The filtrates were slowly cooled down to 5° C. at a rate of 0.1° C./min. Results summarized in Table 21 indicated Form A, and a mixture of Forms A and C were generated.

TABLE 21
Summary of slow cooling experiments

| Experiment ID | Solvent (v:v) | Solid Form |
|---|---|---|
| C4-A1 | EtOH/H₂O (1:1) | Form A |
| C4-A2 | ACN/H₂O (2:1) | Forms A + C |
| C4-A3* | THF/H₂O (9:1) | Forms A + C |
| C4-A4 | DMAc/H₂O (1:3) | Form A |

*no solid was obtained after cooling and evaporation at rt was employed.

### D. Polymorph Screening Experiments

Slurry at Room Temperature: Slurry experiments were conducted at rt in various solvent systems. About 15 mg of starting material was suspended in 0.3 mL of solvents in HPLC vial. After the suspension was stirred magnetically for 3-7 days at rt, the remaining solids were isolated for XRPD analysis. Results summarized in Table 22 indicated that Form B, a mixture of Forms A and C, Form A of low crystallinity, and low crystallinity/amorphous samples were obtained.

TABLE 22
Summary of slurry experiments at room temperature

| Experiment ID | Solvent | Solid Form |
|---|---|---|
| D1-A1 | THF | Amorphous |
| D1-A2 | Toluene | Forms A + C |
| D1-A3 | ACN | Form A (low) |
| D1-A4 | DCM | Form A (low) |
| D1-A5 | EtOAc | Low crystallinity |
| D1-A6 | MTBE | Low crystallinity |
| D1-A7 | Acetone | Amorphous |
| D1-A8 | IPA | Low crystallinity |
| D1-A9 | EtOH | Low crystallinity |
| D1-A10 | MeOH | Form B |
| D1-A11 | DMAc | Amorphous |
| D1-A12 | n-Heptane | Form A + C |

Form A (low): difficult to judge whether they had Form C due to low crystallinity; and Low crystallinity: amorphous + 1 or 2 peak(s).

Slurry at 50° C.: Slurry experiments were also conducted at 50° C. in various solvent systems. About 20 mg of starting material was suspended in 0.3 mL of solvent in HPLC vial. After the suspension was stirred for about 3 days at 50° C., the remaining solids were isolated for XRPD analysis. Results in Table 23 indicated that a mixture of Forms A and C, Form A of low crystallinity and low crystallinity/amorphous samples were generated.

TABLE 23
Summary of slurry experiments at 50° C.

| Experiment ID | Solvent | Solid Form |
|---|---|---|
| D2-A1 | EtOH | Form A (low) |
| D2-A2 | IPA | Low crystallinity |
| D2-A3 | THF | Amorphous |
| D2-A4 | Acetone | Amorphous |
| D2-A5 | EtOAc | Forms A + C |
| D2-A6 | ACN | Form A (low) |
| D2-A7 | Toluene | Forms A + C |

Form A (low): difficult to judge whether they had Form C due to low crystallinity; and Low crystallinity: amorphous + 1 or 2 peak(s).

Slow Cooling: Slow cooling experiments were conducted in two solvent systems. About 20 mg of starting material was suspended in 1.5 mL of solvents in a 4-mL glass vial. The suspension was then heated to 50° C., equilibrated for 3 hrs and filtered using a Nylon membrane (pore size of 0.22 μm). The filtrates were slowly cooled down to 5° C. at a rate of 0.1° C./min. Results summarized in Table 24 indicated Form C was generated.

TABLE 24
Summary of slow cooling experiments

| Experiment ID | Solvent | Solid Form |
|---|---|---|
| D3-A1 | MeOH | Form C |
| D3-A2* | DMAc | Clear |

*clear solution was obtained after cooling or evaporation at RT/50° C. for more than two weeks.

Solid Vapor Diffusion: Solid vapor diffusion experiments were conducted using 8 different solvents. Approximate 12 mg of starting material was weighed into a 4-mL vial, which was placed into a 20-mL vial with 3 mL of volatile solvent. The 20-mL vial was sealed with a cap and kept at rt for ~9 days allowing solvent vapor to interact with sample. The solids were tested by XRPD and the results in Table 25 showed a mixture of Forms A and C, Form A of low crystallinity and low crystallinity samples were generated.

TABLE 25
Summary of solid vapor diffusion experiments

| Experiment ID | Solvent | Crystal Form |
|---|---|---|
| D4-A1 | THF | Low crystallinity |
| D4-A2 | DCM | Forms A + C |
| D4-A3 | DMAc | Low crystallinity |
| D4-A4 | EtOAc | Form A (low) |
| D4-A5 | Toluene | Forms A + C |
| D4-A6 | EtOH | Form A (low) |
| D4-A7 | IPA | Form A (low) |
| D4-A8 | ACN | Form A (low) |

Form A (low): difficult to judge whether they had Form C due to low crystallinity; and Low crystallinity: amorphous + 1 or 2 peak(s).

Anti-solvent Addition: A total of 12 anti-solvent addition experiments were carried out at rt. About 15 mg of starting material was dissolved in ~3.0 mL solvents to obtain a clear solution. The solution was magnetically stirred followed by addition of 0.2~0.5 mL anti-solvent stepwise till precipitate appeared or the total amount of anti-solvent reached 15.0 mL. The obtained precipitate was isolated for XRPD analysis. Results in Table 26 showed that Form A and amorphous were obtained.

TABLE 26
Summary of anti-solvent addition experiments

| Experiment ID | Solvent | Anti-solvent | Solid Form |
|---|---|---|---|
| D5-A1* | MeOH | THF | Form A |
| D5-A2 | | DCM | Amorphous |
| D5-A3 | | Toluene | Amorphous |
| D5-A4 | | MTBE | Amorphous |
| D5-A5* | | EtOAc | Amorphous |
| D5-A6* | | Acetone | Form A |
| D5-A7* | DMAc | THF | Clear |
| D5-A8* | | DCM | Amorphous |
| D5-A9* | | Toluene | Clear |
| D5-A10* | | EtOAc | Clear |
| D5-A11 | | IPA | Amorphous |
| D5-A12* | | ACN | Clear |

*no solid was obtained after anti-solvent addition/5° C. stirring/−20° C. standing and then evaporation at rt was employed; and Clear solutions were observed after evaporation at rt for more than 2 weeks.

Polymer-induced Crystallization: About ~10 mg of starting material was dissolved in 1.0 mL of solvents in a 4-mL glass vial. If no dissolution was achieved, suspensions were filtered using a Nylon membrane (pore size of 0.22 μm) and the filtrates were used for the following steps. Then the visually clear solutions and the filtrates were used for the following steps. 1~2 mg polymer mixtures of PVC and PVP was added into the solutions, and the samples were covered by Parafilm® with 4 pinholes followed by evaporation at rt. Results summarized in Table 27 indicated that Form A, and a mixture of Forms A and C were obtained.

TABLE 27
Summary of polymer-induced crystallization experiments

| Experiment ID | Solvent (v:v) | Crystal Form |
|---|---|---|
| D6-A1 | MeOH | Forms A + C |
| D6-A2* | DMAc | Clear |
| D6-A3 | THF/H₂O (2:1) | Form A (low) |
| D6-A4 | ACN/H₂O (2:1) | Form A |

Form A (low): difficult to judge whether they had Form C due to low crystallinity; and
*clear solution was obtained after evaporation at rt/50° C. for more than two weeks.

### Example 7: Critical Water Activity Study Among Mono-Sodium Salt Forms

Critical water activity (a_{w}) study was performed for mono-sodium salt Form A (monohydrate), Form E (hemihydrate) and Form D (anhydrate) to investigate the form relationship. It was conducted via competitive slurry in THF/H₂O with a series of a_{w} (0~1) at rt. Specifically, ~10 mg staring material was suspended in 0.4 mL THF/H₂O (a_{w} of 0, 0.2, 0.4, 0.6, 0.8 and 1.0) at rt. After equilibrium for 2 hrs, collect the filtrates to the HPLC vials containing Form A and Form D with equivalent mass ratio. After stirring magnetically at rt for 1~2 days, collect the solids for XRPD analysis.

The results summarized in Table 28 indicated that hemihydrate Form E was obtained in THF/H₂O (a_{w} of 0~0.8) at rt, indicating it was more stable than monohydrate Form A and anhydrate Form D at those conditions. Furthermore, Form A was obtained in water, indicating it was more stable than Form D at a_{w} of 1.0 at rt.

TABLE 28
Critical water activity study results
among Forms A/D/E at room temperature

| Experiment ID | Starting Forms | A_{w} (v:v, THF/H₂O) | Final Form |
|---|---|---|---|
| Ex. 7-A1 | Hydrate Form A + | 0.0 (1000:0) | Form E |
| Ex. 7-A2 | Anhydrate Form D | 0.2 (981:19) | Form E |
| Ex. 7-A3 | | 0.4 (956:44) | Form E |
| Ex. 7-A4 | | 0.6 (923:77) | Form E |
| Ex. 7-A5 | | 0.8 (869:131) | Form E |
| Ex. 7-A6 | | 1.0 (0:1000) | Form A |

### Example 8: Process Development for Preparing Compound of Formula (IIa) Via Step a)

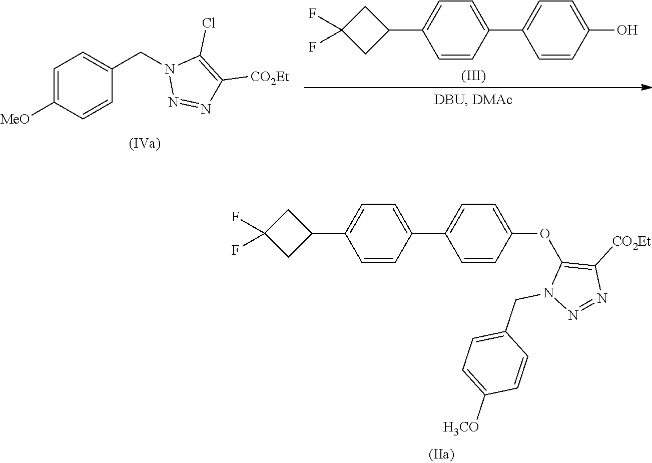

Experiment 8.1. The reaction was conduct with compound (III) (20.9 g), the conversion of compound (III) to compound (IIa) was 88% for 40 h. Compound (IIa) (19.6 g) was obtained as an off-white solid with 90.87% of purity and 55.78% of yield after crystallization with ethanol (10 vol). The mother liquor of ethanol is sticky and difficult to filter.

Experiment 8.2. The reaction was conduct with compound (III) (20.9 g), the conversion of compound (III) to compound (IIa) was 88% for 72 h. Compound (IIa) (16.2 g) was obtained with 95.76% of purity and 46.13% of yield after crystallization with ethanol (5 vol) twice.

Conclusion: Crystallization with ethanol gave a higher yield loss in mother liquor. Yield appeared lower than prior recorded yields (60~65%). A process using column chromatography prior to crystallization was then studied.

Experiment 8.3. The crude Compound (IIa) in an organic phase was purified by column chromatography under the conditions: Step A: 30 g Silica gel, 10 g Silica gel, 45 g EtOAc, and 34 g n-Heptane—1) Charged silica gel to the column; 2) Charged silica gel to another portion of organic phase; 3) Concentrated to no distillate; 4) The concentrate was a sticky oil; 5) Charged EtOAc and n-heptane to the concentrate; 6) Charged the mixture to the column; and Step B: 45 g EtOAc, 34 g n-Heptane, 34 g n-Heptane, 3.6 g EtOAc, and 5.4 g n-Heptane—7) Washed the column with the mixture of EtOAc and n-heptane; 8) Combined the filtrate; 9) Concentrated the filtrate to 10~11 Vol and agitate for 3~4 h to precipitate the solid; 10) Dropped n-heptane in 1 h and agitate for 2~3 h; 11) Filtered the crystallization solution; 12) Washed the filter cake with the mixture of EtOAc and n-heptane. Compound (IIa) (11.2 g) was obtained as an off-white with 89.9% of purity and 63.7% of yield. The method of column chromatography was further studied to improve the purity.

Experiment 8.4. The reaction was conducted with compound (III) (20.5 g). The conversion of compound (III) to compound (IIa) was 88% for 40 h. The crude Compound (IIa) in an organic phase was purified by column chromatography under the conditions: Step A: 50 g Silica gel (5×), 70 g n-Heptane, 30 g EtOAc, and 10 g n-Heptane following 1) to 6) of Experiment 8.3; Step B: 135 g EtOAc (13.5×), 102 g n-Heptane (10.2×), 34 g n-Heptane, 3.6 g EtOAc, and 5.4 g n-Heptane following 7) to 12) of Experiment 8.3; and Step C: 13) Dried the filter cake under vacuum at 45° C. and 14) 10.55 g off-whiter solid was no btained. Compound (IIa) (10.55 g) was obtained as an off-white with 94.08% of purity and 61.8% of yield.

Experiment 8.5. Compound (IIa) was scaled up starting with compound (IVa) (45 g) and compound (III) (46.7 g) according to the steps described below. The conversion of compound (III) to compound (IIa) was 89% at 90° C. for 40 h. Compound (IIa) (49.11 g) was obtained as an off-white solid with 95.21% of purity and 62.0% yield.

Reaction: Charged compound (IVa) (45 g, 1 eq.), compound (III) (46.7 g, 1 eq. corrected by assay), DMAc (58.5 g), and DBU (31.5 g, 1.35 eq.) to the reactor; Heated to 90° C. and agitated for 40 h; and a sample was tacked for IPC by HPLC.

Preparation: Charged 135 g NH₄Cl (3×) and 450 g water (10×) to another reactor; Agitated to give a clear solution and warm to 15~25° C.; Charged 22.5 g LiCl (0.5×) and 427.5 g water (9.5×) to another reactor; and Agitated to give a clear solution and cooled to 15~25° C.

Quenching: Charged 810 g EtOAc (18×); Charged NH₄Cl aq. and agitated for 30~40 min; Separated two phase (aqueous phase was discarded and the organic phase was stored); Charged LiCl aq. to the organic phase; and Separated two phase (aqueous phase was discarded and the organic phase was stored.

Concentration 1: Concentrated the organic phase to 2~3 Vol under vacuum at 30~40° C.; and Charged 90 g EtOAc (2×) and 45 g n-heptane (1×) to the concentrate.

Column chromatography: Charged 225 g silica gel (5×) to the column; Washed the column with 306 g n-heptane (6.8×), the filtrate was discarded; Charged the concentrate mixture to the column; Washed the column with the mixture of 607.5 g EtOAc (13.5×) and 459 g n-heptane (10.2×); and Combined the filtrate.

Concentration 2: Concentrated the filtrate to 10~11 Vol at 40~50° C. under vacuum; Cooled to 20~30° C. and agitated for 2~4 h to solid precipitate; Dropped n-heptane 153 g (3.4×) and agitated for 2~3 h; Filtered the mixture; Washed the filter cake with 16.2 g EtOAc (0.36×) and 25.2 g n-heptane (0.56×).

Drying: Dried the filter cake at 40~50° C. under vacuum for 12~16 h; Took a sample for IPC; and compound (IIa) (49.11 g) was obtained with an off-white solid with 95.210% of purity and 62% of yield.

### Example 9: Process Development for Preparing Compound of Formula (Ia) Via Step b)

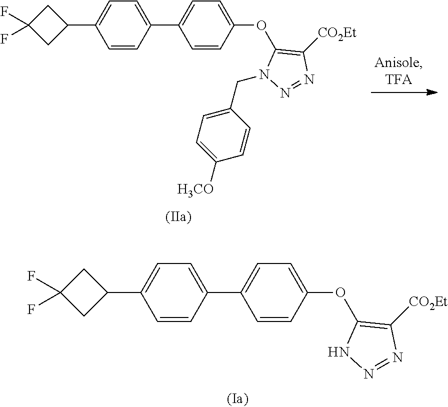 [SMILES: CCOC(=O)c1nnn(Cc2ccc(OC)cc2)c1Oc1ccc(-c2ccc(C3CC(F)(F)C3)cc2)cc1 ; CCOC(=O)c1nn[nH]c1Oc1ccc(-c2ccc(C3CC(F)(F)C3)cc2)cc1]

Reactions: Charged compound (IIa) (10 g), anisole (6.25 g, 3 eq.) and 32.6 g TFA (32.6 g, 15 eq.) to a dry flask; Heated to 55~60° C. for 3 h and sampled for IPC. (HPLC: (IIa) 0.10% of compound (IIa)); and Cooled the reaction mixture to 20~30° C.

Concentration: Charged 34 g n-heptane (3.4×) to the mixture; Concentrated the mixture at 20~30° C. under vacuum to 6~7 Vol; Charged 34 g n-heptane (3.4×) to the mixture; Concentrated the mixture at 30~40° C. under vacuum to 6~7 Vol.; and Cooled the concentrate to 20~25° C.

Filtration: Filtered the mixture; and Soaked and washed the filter cake with the mixture of 5.8 g toluene (0.58×) and 9 g n-heptane (0.9×) twice.

Drying: Dried the wet cake at 45° C. under vacuum for 16 h and sampled for IPC; and compound (Ia) (6.7 g) was obtained as an off-white solid with 99.13% of purity and 87.2% of yield.

The process development focused on improving a purification process to address a few impurities observed (e.g., RT 6.2 min, RT 8.6 min, and/or RT 12.2 min by HPLC).

Slurry of Crude Compound (Ia) with Toluene and n-Heptane

Experiment 9.1. Compound (Ia) was prepared according to the procedure as described above starting with compound (IIa) (100 g). After slurrying with the mixture of toluene and n-heptane thrice, compound (Ia) (63.6 g) was obtained as an off-white solid with 82.7% of yield and 98.05% of purity. The extended time of slurry with toluene/n-heptane solution (1:1) provided no improvement on the purity of compound (Ia).

Experiment 9.2. The material of Experiment 9.1 (5 g) was further slurried with toluene for 16 h. Compound (Ia) (4.65 g) was obtained as a white solid with 98.94% of purity and 93% of yield. The toluene slurry did not remove two impurities of compound (Ia) (e.g., RT 8.6 min and/or RT 12.2 min by HPLC).

Recrystallization of Crud Compound (Ia) with Toluene or Ethanol

Experiment 9.3. Compound (Ia) was prepared according to the procedure as described above starting with compound (IIa) (10 g). Recrystallization the crude compound (Ia) with toluene (3×), compound (Ia) (16.15 g) was obtained as a white solid with 99.13% of purity and 70.0% of yield. The impurity at RT 8.6 min remained no change (0.28%).

Compound (Ia) was prepared according to the procedure as described above starting with compound (IIa) (100 g). The crude compound (Ia) was divided into 5 portions to screen the work-up process.

Experiment 9.4. The work-up was to recrystallize with 1.1× ethanol followed by wash with 0.35× ethanol at 15° C. Compound (Ia) (12.4 g) was obtained with 99.70% of purity and 810% of yield. This work-up process was incorporated in Experiment 9.6 to test reproducibility.

Experiment 9.5. The work-up was to recrystallize with 1.5× ethanol followed by wash with 0.5× ethanol at 15° C. Compound (Ia) (22.68 g) was obtained with 99.17% of purity and 74% of yield.

Experiment 9.6. Compound (Ia) was scaled up starting with compound (IIa) (40 g) according to the steps described below. Compound (Ia) (20.61 g) was obtained as an off-white solid with 99.78% of purity and 67% of yield. The lower yield may be contributed to a loss of compound (Ia) to a high TFA residue after concentration.

Reaction: Charged compound (IIa) (40 g), anisole (25 g, 3 eq.) and TFA (131.6 g, 15 eq.) to a dry jacket reactor; Heated to 55~60° C. and agitated for 3 h, sampled for IPC by HPLC for completion; and Cooled the reaction mixture to 20~30° C.

Concentration: Charged 136 g n-heptane (3.4×) to the mixture; Concentrated the mixture at 20~30° C. under vacuum to ~6 Vol.; Charged 136 g n-heptane (3.4×) to the mixture; Concentrated the mixture at 30~40° C. under vacuum to ~6Vol.; and Filtered the mixture.

Slurry: Charged the filter cake to the reactor; Charged 48 g toluene (1.2×) and 112 g n-heptane (2.8×) and agitated to give a slurry; Heated to 65~70° C. and agitated for 4 h; Cooled to 20~25° C. in 4 h, then agitated for 3~4 h; and Filtered the slurry.

Crystallization: Charged the filter cake to a dry reactor; Charged 44 g ethanol (1.1×); Heated to 70° C. and agitated to give a clear solution; Cooled to 55° C. in 1 h and agitated for 2~3 h to precipitate the solid; Cooled to 15° C. in 6~8 h and agitated for 2~3 h at 15° C.; and Filtered the crystallization solution and washed the filter cake with 14 g ethanol (0.35×).

Drying: Dried the filter cake at 45° C. under vacuum for 24 h; Took a sample for IPC by GC for ethanol residue; and compound (Ia) (20.61 g) was obtained with a white to off-white solid whit 99.78% of purity and 67% of yield, meeting the product specification.

Experiment 9.7. In order to ensure the yield of compound (Ia), an IPC of TFA residue was set after concentration. Based on the mass-balance and solubility, the TFA residue limit of the IPC was set to no more than 4.0%. Compound (Ia) was prepared starting with compound (IIa) (30 g) according to the steps described above with TFA residue of about 3.8% by an IPC. Compound (Ia) (18.46 g) was obtained as an off-white solid with 99.67% of purity and 88.0% of yield.

### Example 10: Process Development for Preparing Compound 1b Via Steps c) and d)

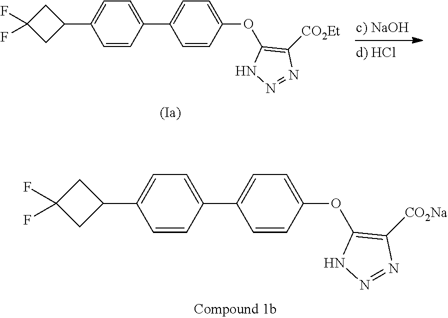

Reaction: Charged NaOH (1.65 g) and purified water (39.6 g), agitated to dissolve and cooled to 20~30° C.; Charged compound (Ia) (5.5 g); Heated to 80° C. and agitated for 1 h; Cooled to 20~30° C. and agitated for 16 h, then sampled for IPC for completion.

Acidification: Dropped 6 M aq. HCl (4.2 g) to precipitate out solid (pH 9); Dropped 2 M aq. HCl to adjust pH to 7; and Agitated for 2 h and filtration.

Drying: Dried at 40° C. under vacuum for 16 h; and sampled for IPC (Water content; Result water content 3.8%). Compound 1b (5.34 g) was obtained as a white solid with 99.24% of purity and 98.5% of yield.

The process development focused on improving the purity of compound 1b by tuning the acidification step.

Experiment 10.1. The reaction was conducted with compound (Ia) (30 g) according to the procedure described above. The conversion of compound (Ia) to compound 1b was 98.26% at 80° C. after 3 h. The acidification was conducted as follows: 1) Dropped 6 M HCl to precipitate out solid (pH 9.6); Dropped 2 M HCl (pH: no change); and Sample had 99.25% purity; and 2) Dropped 2 M HCl until pH from 9.6 to 7.8; Filtered the mixture; Washed the filter cake with purified water twice; Washed the filter cake with n-heptane twice; and Sample had 99.12% purity. After drying at 40° C. under vacuum for 16 h, compound (1b) (29.05 g) was obtained as a white solid with 99.24% of purity and 98.34% of yield. The impurity at RT 5.6 min appeared to increase when the amount of aq. HCl aq. was added. Optimal pH range appeared to be from about 7 to about 8 at the end of acidification.

Experiment 10.2. The reaction was conducted with compound (Ia) (20 g) according to the procedure described above. The conversion of compound (Ia) to compound 1b was 99.41% at 80° C. after 3 h. Wet compound 1b (20.14 g) was obtained as a white solid with 99.86% of purity and 102.25% of yield.

Reaction: Charged NaOH (6 g) and purified water (144 g), dissolved and cooled to 20~30° C.; Charged compound (Ia) (20 g); Heated to 80° C. and agitated for 3 h and a sample was taken for IPC by HPLC for completion; and Cooled to 20~30° C.

Filtration 1: Filtered the reaction solution and washed the filter cake with purified water (1×).

Acidification: Dropped 6 M HCl (16.8 g) to precipitate the solid (pH: 9~10) at 20~40° C.; Dropped 2 M HCl till pH 7~8 at 20~40° C.; and Stirred for 2 h at 10~30° C.

Filtration 2: Filtered the mixture; Washed the filter cake with 20 g x 3 purified water thrice; and Washed the filter cake with 20 g x 2 n-heptane twice.

Drying: Dried at 35~45° C. under vacuum for 16 h. A sample was taken for IPC by Coulomb moisture (Limit: Water content no more than 6.0%). Compound 1b (20.14 g D198) was obtained as a white solid with 102.25% of purity and 99.86% of yield, meeting the product specification.

### Example 11: Process Development for Preparing 4′-(3,3-Difluorocyclobutyl)-[1,1′-biphenyl]-4-ol (Compound (III))

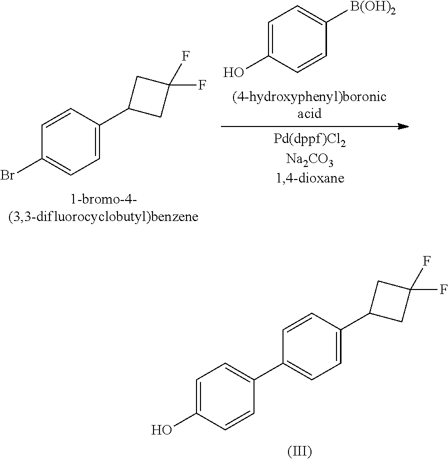

The process development focused on optimization of the process of remove Pd residue.

Experiment 11.1. The reaction was conducted with 1-bromo-4-(3,3-difluorocyclobutyl)benzene (50 g). The conversion to compound (III) was 80.41%. Thiourea resin was charged before discoloration, the mixture was agitated for 20 h at 45~50° C., Pd residue decreased from 48.5 ppm to 26.4 ppm with prolonging stirring time from 4 h to 20 h. Compound (III) (29.2 g) was obtained as a white solid with 99.65% purity and 92.35% yield, and Pd residue was not detected. Compound (III) met the product specification (Pd residue is no more than 20 ppm).

Experiment 11.2. The reaction was conducted with a solution of 1-bromo-4-(3,3-difluorocyclobutyl)benzene in 1.4-dioxane (109.5 g of the solution, 1-bromo-4-(3,3-difluorocyclobutyl)benzene—15 g by concentration and 12.29 g by an assay), according to the steps below. The reaction went to completion, monitored by an IPC.

Reaction: Charged a 1.4-dioxane solution of 1-bromo-4-(3,3-difluorocyclobutyl)benzene (109.5 g of the solution, 15 g of 1-bromo-4-(3,3-difluorocyclobutyl)benzene), (4-hydroxyphenyl)boronic acid (12.6 g, 1.5 eq.), Na₂CO₃ (9.6 g, 1.5 eq.) and 101.2 g water (101.2 g) into the reactor; Degased with N₂ for 2~3 h; Charged Pd(dppf)Cl₂ (0.23 g, 0.005 eq.); Heated to 45~55° C. and agitated for 20 h. A sample was taken for IPC to ensure completion.

Washing: Cooled the reaction solution to 20~30° C.; Charged EtOAc (180 g) and water (100 g); Filtered the mixture and washed the filer cake with EtOAc (22.5 g); Separated the filtrate; Concentrated to 2~3 Vol at 40~50° C. under vacuum; Charged EtOAc (135 g) and concentrated to 9~10 Vol.

Discoloration: Charged thiourea resin (5 g); Heated to 45~50° C. and agitated for 24 h. A sample was taken for Pd residue (detected as 18 ppm); Charged activated carbon (7.5 g) to the mixture and agitated for 30~60 min at 40~50° C.; Filtered the mixture at 40~50, and washed the filter cake with EtOAc (112.5 g); Concentrated the filtrate to 1~2 Vol at 40~50° C.; Charged 1.4-dioxane (75 g) to the concentrate and concentrated to 2~3 Vol at 40~50° C. under vacuum; Charged 1.4-dioxane (75 g) to the concentrate and concentrated to ~2 Vol at 40~50° C. under vacuum.

Recrystallization: Dropped n-Heptane (120 g) to the concentrate at 40~50° C.; Cooled to 0~5° C. slowly and agitated for 2~3 h; Filtered and washed the filter cake with 1.5 g 1,4-dioxane (1.5 g) and n-heptane (12 g); and Washed the filter cake with water (30 g) four times.

Drying: Dried at 50~60° C. under vacuum for 12~16 h. A sample was taken for IPC (Water content no more than 1.0%). Compound (III) (12.5 g) was obtained as an off-white to light brown solid with 97.78% of purity and 96.6% of yield, meeting the product specification.

### Example 12: Process for Preparing 4′-(3,3-Difluorocyclobutyl)-[1,1′-biphenyl]-4-ol (Compound (III))

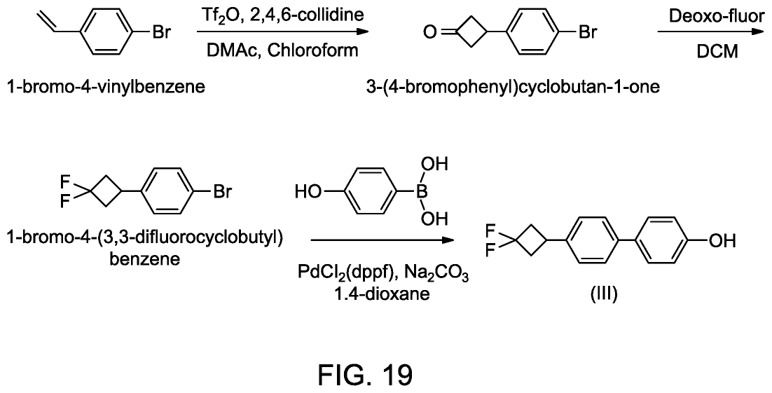

Compound (III) was prepared according to the Scheme of FIG. 19.

### A. 3-(4-bromophenyl)cyclobutan-1-one

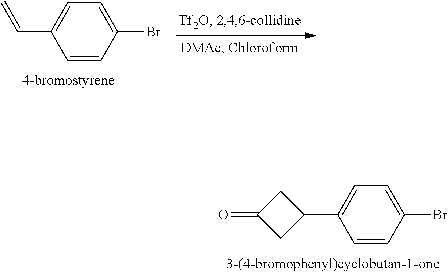

Reaction 1: DMAc (19.5 kg, 1.2 eq.) and chloroform (340.2 kg) were charged to reactor. The mixture was cooled to 4~8° C. TF₂O (62.9 kg, 1.2 eq.) was added dropwise while maintaining the reaction temperature between 4~8° C. The white thick reaction mixtures were stirred at 4~8° C. for 30-40 min. A mixture of 4-bromostyrene (33.9 kg, 1 eq.), chloroform (68 kg), 2, 4, 6-collidine (28.2 kg, 1.25 eq.) were added dropwise while maintaining the reaction temperature between 4~8° C. The reaction mixture was heated to 60~65° C. and stirred 44 h. A sample was taken for IPC by HPLC (4-bromostyrene: 19.75%, 3-(4-bromophenyl)cyclobutan-1-one (referred hereafter as ketone): 44.52%).

Quenching: The reaction mixture was cooled to 20~30° C. Water (268.4 kg) was charged. The reaction mixture was heated to 60~65° C. and stirred 4 h. The reaction mixture was cooled to 20~30° C. After separation, organic phase was stored and aqueous phase was abandoned. Water (170.2 kg) was charged to organic phase. After separation, organic phase was stored and aqueous phase was abandoned.

Reaction 2: Water (88.4 kg), sodium bisulfite (38.4 kg) and EtOH (136 kg) were charged to the organic phase at reactor. The reaction was stirred 16 h at 20~30° C.

Filtration 1: The reaction solution was filtered. The filter cake was washed twice with DCM (112.6 kg).

Reaction 3: DCM (279.7 kg) and wet cake were charged to reactor and stirred 30 min. 1 N NaOH (169.5 kg) was added dropwise the reaction solution and stirred 6 h. Separation: Organic phase was stored and aqueous phase was treated with DCM. DCM (54.2 kg) was charged to the aqueous phase at reactor. After separation, the organic phase was combined and aqueous phase was discarded.

Drying: Na₂SO₄ (19.3 kg) was charged to the organic phase. The mixture was agitated for 12 h at 20~30° C.

Filtration 2: The mixture was filtered. The filter cake was washed twice with DCM (27.1 kg).

Concentration: The organic filtrate was concentrated to 2~3 Vol under vacuum at 25~35° C. A solution of 3-(4-bromophenyl)cyclobutan-1-one in DCM (129.3 kg) was obtained with a purity of 95.81%. The content of the ketone in DCM was determined to be 9.9% by an assay. After correction by the assay, 3-(4-bromophenyl)cyclobutan-1-one (12.8 kg) was obtained in a yield of 29.8%.

### B. 1-bromo-4-(3,3-difluorocyclobutyl)benzene

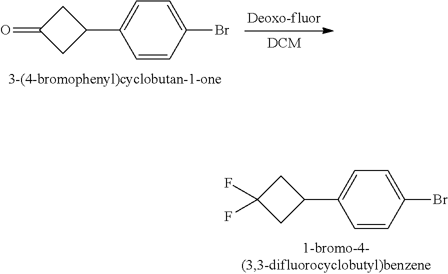 [SMILES: O=C1CC(c2ccc(Br)cc2)C1 ; FC1(F)CC(c2ccc(Br)cc2)C1]

Reaction: A solution of 3-(4-bromophenyl)cyclobutan-1-one in DCM (129.1 kg) (12.8 kg the ketone corrected by the assay) and DCM (40 kg) were charged into the reactor. The reaction mass was cooled to 0~5° C. Deoxo-fluor (22.8 kg, 1.8 eq.) was charged into the reaction tank under 0~10° C. The reaction mass was heated to 28~32° C. and agitated for 22 h. A sample was taken for IPC by HPLC (result: the ketone=0.79%).

Quenchin: The reaction mass was cooled to 0~5° C. Water (1.92 kg) was slowly dropped for 2 h at 0~10° C. Water (46.08 kg) was charged at 0~10° C. 20% aq. NaOH (29.46 kg) was charged at 0~20° C.

Extraction: The quenched mass was agitated for 20~30 min followed by standing for 1~2 h. after separation, organic phase was stored and aqueous phase was transferred to the reaction tank. DCM (34.05 kg) was charged into aqueous phase. The mixture was agitated for 20~30 min followed by standing for 1~2 h. After separation, organic phase was combined and aqueous phase was discarded

Concentration 1: The organic phase was concentrated to no fraction at 20~25° C. n-Heptane (17.4 kg) was charge to dilute the concentrate to obtain a solution of 1-bromo-4-(3,3-difluorocyclobutyl)benzene in n-heptane.

Filtration 1: Silica gel (23.0 kg) was charged into a filter. The above n-heptane solution was filtered through silica gel. The filter cake was washed with n-heptane (156.67 kg).

Concentration 2: Filtrate was concentrated to no fraction at 40~50° C.

Impurity degradation: The residue after concentration 2 was heated to 150° C. for 23 h. A sample was taken for IPC by HPLC (specification: vinyl fluoride impurity ≤0.2%) (Result: vinyl fluoride impurity: N/D). n-Heptane (17.4 kg) was charged to dilute the solution.

Filtration 2: Silica gel (23.0 kg) was charged into filter. The n-heptane solution from the first filtration was filtered through silica gel. The filter cake was washed with n-heptane (156.67 kg).

Concentration 3: Filtrate was concentrated to no distillate at 40~50° C. 1.4-Dioxane (55.0 kg) was charged to the concentrate. A 1.4-dioxane solution of 1-bromo-4-(3,3-difluorocyclobutyl)benzene (60.2 kg) (6.5 kg 1-bromo-4-(3,3-difluorocyclobutyl)benzene corrected by an assay) was obtained with a purity of 94.6% and a yield of 46.3%.

### C. 4′-(3,3-difluorocyclobutyl)-[1,1′-biphenyl]-4-ol (Compound (III))

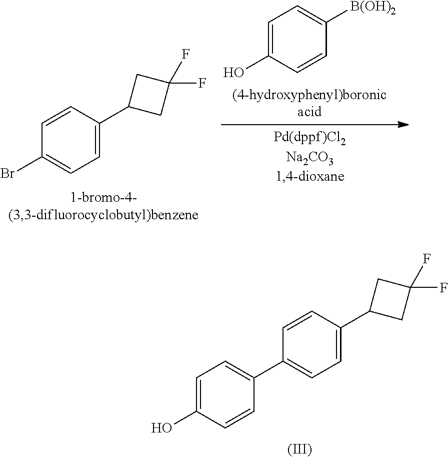

Reaction: A 1.4-dioxane solution of 1-bromo-4-(3,3-difluorocyclobutyl)benzene (60.2 kg) (6.5 Kg 1-bromo-4-(3,3-difluorocyclobutyl)benzene corrected by the assay), Na₂CO₃ (4.5 kg, 1.5 eq.), water (46.8 kg), and (4-hydroxyphenyl)boronic acid (5.9 kg, 1.5 eq.) were charged to the reactor in order. The reaction mass was degassed with N₂ for 3 h. Pd(dppf)Cl₂ (0.1065 kg, 0.005 eq.) was charged with N₂ protection. The reaction was warmed to 40~50° C. and agitated for 20 h. A sample was taken for IPC by HPLC (Result: 3-(4-bromophenyl)cyclobutan-1-one: 2.52%).

Separation: After IPC1, the reaction mixture was cooled to 15~25° C. Water (44.6 kg) and EtOAc (84.6 kg) were charged and agitated for 30~45 min. The mixture was filtered and the filter cake was washed with EtOAc (10.5 kg). The filtrate was separated, the organic phase was kept, and the aqueous phase was stored.

Concentration 1: The organic phase was concentrated to no distillate under vacuum at 40~50° C. EtOAc (52.5 kg) was charged and concentrated to 2~3 Vol under vacuum at 40~50° C. EtOAc (63.0 kg) was charged and concentrated to 9~10 Vol under vacuum at 40~50° C.

Discoloration: Thiourea resin (2.1 kg) was charged to the concentrate and agitated for 24 h at 45~55° C. A sample was taken for IPC (specification: Pd residue NMT 20 ppm; result: Pd residue: 14 ppm). Activated carbon (3.5 kg) was charged and agitated for 1 h. The solution was filtered at 40~50° C. The filter cake was washed with hot EtOAc (53.5 kg) which was 45° C. The filtrate was combined.

Concentration 2: The filtrate was concentrated to 1~2 Vol under vacuum at 40~50° C. 1.4-Dioxane (35 kg) was charged and concentrated to 2~3 Vol under vacuum at 45~55° C. 1.4-Dioxane (35 kg) was charged and concentrated to ~2 Vol under vacuum at 45~55° C.

Recrystallization: n-Heptane (26.51 kg) was dropped at 45~55° C. The crystallization liquid was cooled to 0~5° C. over 16 h and agitated for 4~6 h at 0~5° C.

Filtration: The crystallization liquid was filtered at 0~5° C. The filter cake was washed with a mixture of 1.4-dioxane (1.0 kg) and n-heptane (8.8 kg). The filter cake was washed with water (14 kg) four times.

Drying: The wet product was dried at −0.09 MPa and 50~60° C. (55° C. recommended) for 24~36 h. A sample was taken for water content. (specification: water content NMT 1.0%; result: water content: 0.21%).

Packed and weighed the production: 6.6 Kg Compound (III) (6.6 kg, 5.56 kg corrected by an assay (84.2%)) was obtained as light-brown solid with a purity of 96.24% and a yield of 75%.

### Example 13: Process for Preparing Ethyl 5-chloro-1-(4-methoxybenzyl)-1H-1,2,3-triazole-4-carboxylate (Compound (IVa))

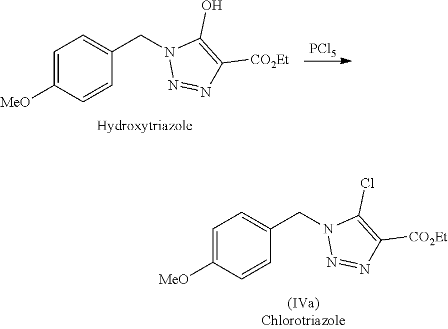 [SMILES: CCOC(=O)c1nnn(Cc2ccc(OC)cc2)c1O ; "PCl5" ; CCOC(=O)c1nnn(Cc2ccc(OC)cc2)c1Cl]

Reaction: Toluene (143 kg), PCl₅ (11.86 kg, 1.2 eq.) were charged into the reactor. The reaction solution was heated to 55~65° C. to give a clear solution. Hydroxytriazole (13 kg, 1.0 eq.) was divided in to 10 portions and charged into the reaction solution. The reaction solution was agitated for 1 h. A sample was taken for IPC by HPLC (hydroxytriazole: N/D).

Quenching: Water (195 kg) was charged to the quenching reactor. NaHCO₃ (28 kg, 7.0 eq.) was charged and agitated for 30~40 min. Toluene (13.0 kg) was charged. The quenching solution was cooled to 0~10° C. The reaction mass was dropped to the quenching solution at 0~10° C. The quenching mass was warmed to 15~25° C. After separation, the aqueous phase was stored and the organic phase was transferred to the reactor.

Concentration: The organic phase was concentrated to 2~4 Vol at 45~50° C.

Crystallization: n-Heptane (39.0 kg) was charged to the concentrate. Compound (IVa) seed (65 g) was charged to the concentrate. The mixture was agitated for 12~16 h at 15~25° C. till a large amount of solid appeared. n-Heptane (117.0 kg) was dropped to the crystal mass for 1~2 h. The resulting mixture was agitated for 2~3 h at 15~25° C.

Filtration: The crystal mass was filtered. The filter cake was washed with a mixture of toluene (2.4 kg) and n-heptane (9.4 kg).

Drying: The filter cake was dried under vacuum at 40~45° C. for 12 h and a sample was taken for IPC (result: toluene residue 110 ppm; n-heptane residue 102 ppm; water content 0.3% by weight). Compound (IVa) (9.8 kg) was obtained as a pink solid with a purity of 95.7% and a yield of 70.6%.

### Example 14: Process for Preparing Sodium 5-((4′-(3,3-difluorocyclobutyl)-[1,1′-biphenyl]-4-yl)oxy)-1H-1,2,3-triazole-4-carboxylate (Compound 1b)

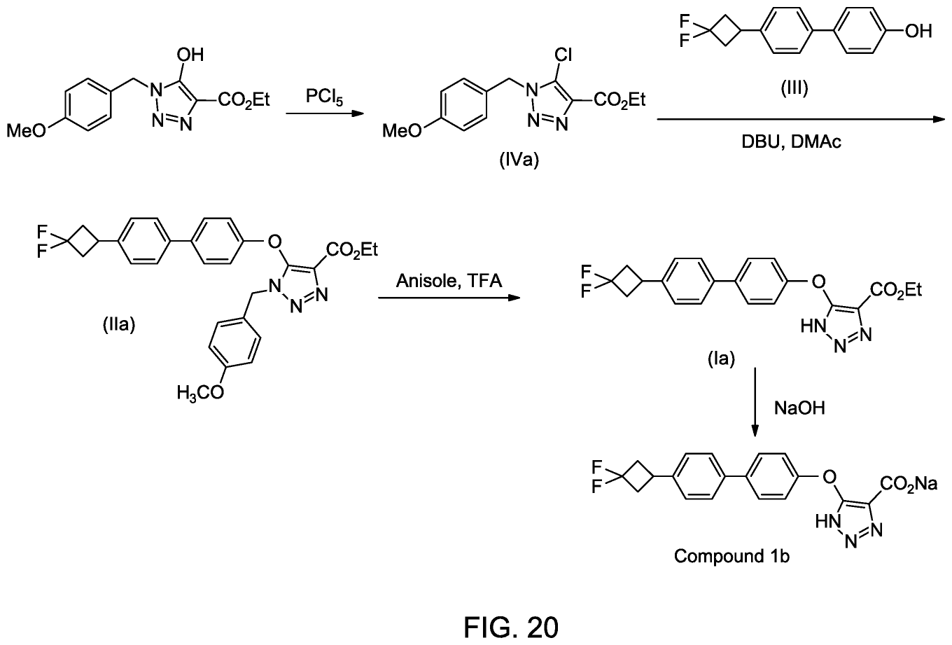

Compound 1b was prepared according to the synthetic scheme of FIG. 20, starting from compounds of formula (III) and (IVa), via steps a) to d).

### Step a): ethyl 5-((4′-(3,3-difluorocyclobutyl)-[1,1′-biphenyl]-4-yl)oxy)-1-(4-methoxybenzyl)-1H-1,2,3-triazole-4-carboxylate (Compound (IIa)

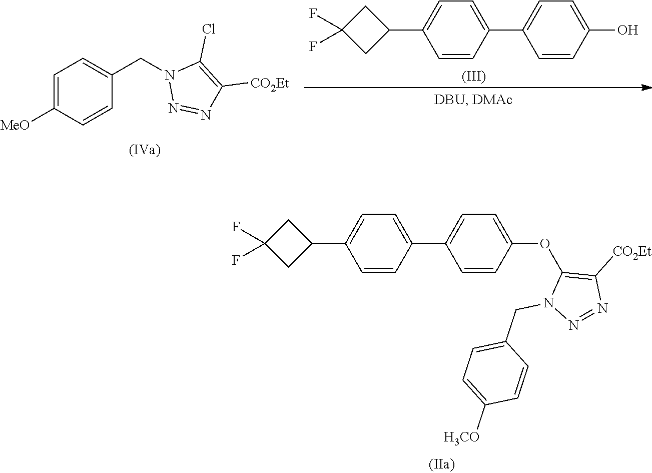

Reaction: Compound (IVa) (6.15 kg, 1.0 eq.), compound (III) (6.2 kg, 1.0 eq.), DMAc (8.2 kg) and DBU (4.4 kg) were charged to the reactor. The reaction mass was heated to 85~90° C. and agitated for 40~44 h. A sample was taken for IPC by HPLC after 48 h. (the conversion of compound (III) to compound (IIa) was 88.5%).

Washing: After IPC, the reaction mixture was cooled to 15~25° C. Water (63.0 kg) and NH₄Cl (18.9 kg) were charged in another reactor and agitated to give a clear solution. EtOAc (113.4 kg) and aq. NH₄Cl were charged and agitated for 30~45 min. After standing for 40~60 min followed by separation, the organic phase was kept in the reactor and the aqueous phase was stored. Water (60.0 kg) and LiCl (3.2 kg) were charged in another reactor and agitated to give a clear solution. Aq. LiCl was charged to the organic phase and agitated for 40~60 min. After standing for 40~60 min followed by separation, the organic phase was kept in the reactor and the aqueous phase was stored.

Concentration 1: The organic phase was concentrated to 1~2 Vol under vacuum at 40~50° C.

Column chromatography: EtOAc (12.6 kg) and n-heptane (6.3 kg) were charged to the concentrate and agitated to uniform. Silica gel (31.5 kg) was charged to column. The column was washed with n-heptane (176 kg×3). The concentrate mixture was charged to the column. The column was washed with a mixture of EtOAc (85.0 kg) and n-heptane (64.3 kg) to elute out compound (IIa). The filtrate were combined and transferred to the crystallizer.

Concentration 2: The filtrate was concentrated to 10~11 Vol under vacuum at 40~50° C.

Crystallization: The concentrate was cooled to 15~25° C. over 2 h and agitated for 12~16 h. n-Heptane (21.4 kg) was dropped to the crystallization mixture over 1~2 h and agitated for 2~3 h.

Filtration: The crystallization mixture was filtered. The filter cake was washed with a mixture of EtOAc (2.3 kg) and n-heptane (3.5 kg).

Drying: The wet product was dried at −0.09 PMa and 40~45° C. for 26 h. A sample was taken for IPC by GC (EtOAc residue: 3049 ppm, n-Heptane residue: 1007 ppm).

Packed and weighed the production: Compound (IIa) (7.86 kg) was obtained as an off-white solid with a purity of 88.13% and a yield of 75.0%.

### Step b): ethyl 5-((4′-(3,3-difluorocyclobutyl)-[1,1′-biphenyl]-4-yl)oxy)-1H-1,2,3-triazole-4-carboxylate (Compound (Ia))

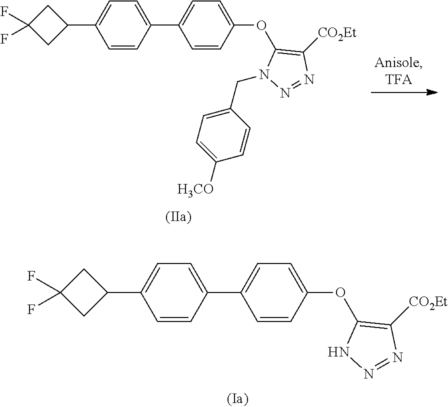 [SMILES: CCOC(=O)c1nnn(Cc2ccc(OC)cc2)c1Oc1ccc(-c2ccc(C3CC(F)(F)C3)cc2)cc1 ; CCOC(=O)c1nn[nH]c1Oc1ccc(-c2ccc(C3CC(F)(F)C3)cc2)cc1]

Reaction: Anisole (4.8 kg, 3.0 eq.), compound (IIa) (7.6 kg, 1.0 eq.), and TFA (25.0 kg, 15 eq.) were charged to the reactor in order. The reaction mixture was heated to 55~60° C. and agitated for 3 h. A sample was taken for reaction IPC (compound (IIa) residue: 0.05%).

Concentration: After IPC, the reaction mixture was cooled to 15~25° C. n-Heptane (25.8 kg) was charged to the reaction solution. The mixture was concentrated to 6~7 Vol under vacuum at 20~30° C. n-Heptane (25.8 kg) was charged to the reaction solution. The mixture was concentrated to 6~7 Vol under vacuum at 30~40° C. A sample of supernatant was taken for concentration IPC (HPLC result: TFA residue 2.8%).

Filtration: The mixture was filtered with the filter dryer.

Slurry: Toluene (9.1 kg) and n-heptane (21.3 kg) were charged to the reactor. The filter cake was charged to the mixture. The mixture was heated to 65~70° C. and agitated for 4~6 h. The mixture was cooled to 20~30° C. (10° C./h) and agitated for 2~3 h. The mixture was filtered with the filter dryer.

Crystallization: ethanol (8.4 kg) was charged to the reactor. The filter cake was charged to the reactor. The mixture was heated to 65~75° C. to give a clear solution. The mixture was cooled to 50~60° C. and agitated for 2~4 h till a large amount of solid precipitate. The mixture was cooled to 10~20° C. (5° C./h) and agitated for 3~4 h. The mixture was filtered with the filter dryer. The filter cake was washed with ethanol (2.7 kg).

Drying 1: The filter cake was dried at 40~45° C. under vacuum for 12~48 h. A sample was taken for drying IPC (GC-HS result: ethanol residue—0.33%; compound (Ia)—98.41%; impurity at RRT 0.94-0.37%; and PMB-anisole—0.49%).

Crystallization 2: Ethanol (7.9 kg) was charged to the reactor. The crude compound (Ia) was charged to the reactor. The mixture was heated to 65~75° C. to give a clear solution.

The mixture was cooled to 50~60° C. and agitated for 2~4 h till a large amount of solid precipitate. The mixture was cooled to 10~20° C. (5° C./h) and agitated for 3~4 h. The mixture was filtered with the centrifuge. The filter cake was washed with ethanol (2.6 kg).

Drying 2: The filter cake was dried at 40~45° C. under vacuum for 12~48 h. A sample was taken for drying IPC (GC-HS result: ethanol residue—0.14%; compound (Ia)—99.41%; impurity at RRT 1.04-0.16%; and PMB-anisole—0.10%).

Packed and weighed the production: Compound (Ia) (3.79 kg) was obtained as an off-white solid with a purity of 99.67% and a yield of 65.0%.

### Steps c) and d): sodium 5-((4′-(3,3-difluorocyclobutyl)-[1,1′-biphenyl]-4-yl)oxy)-1H-1,2,3-triazole-4-carboxylate (Compound 1b)

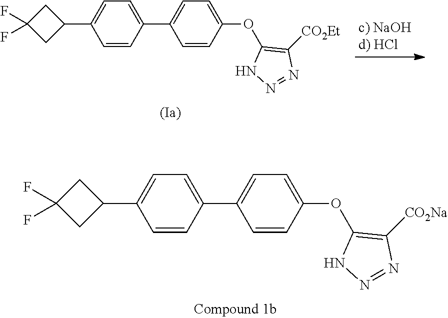

Preparation: Concentrated hydrochloric acid (1.6 kg) and purified water (1.6 kg) were charged to the reactor. The mixture was transferred to the barrel, weighed and temporarily stored as 6 M aq. HCl. Concentrated hydrochloric acid (3.2 kg) and purified water (0.64 kg) were charged to the reactor. The mixture was transferred to the barrel, weighed and temporarily stored as 2 M aq. HCl.

Reaction: Purified water (27.2 kg) and NaOH (1.15 Kg, 3.0 eq.) were charged to the reactor and agitated to dissolve. The solution was cooled to 20~30° C. Compound (Ia) (3.78 kg, 1.0 eq.) was charged to the reactor. The mixture was heated to 75~85° C. and agitated for 3~4 h. A sample was taken for reaction IPC (HPLC result: compound (Ia) N/D).

Filtration 1: After IPC, the reaction mixture was cooled to 15~25° C. The reaction solution was filtered with precision filter. The precision filter was washed with purified water (3.8 kg). The filtrate was combined and transferred to the reactor in the clean area.

Acidification: 6 M aq. HCl was dropped to the filtrate to adjust pH: 9~10 and precipitated the solid of Compound 1b at 20~35° C. 2 M aq. HCl was dropped to adjust pH: 7~8 at 20~35° C. The mixture was agitated for 2 h at 20~35° C.

Filtration 2: The mixture was filtered with the filter dryer. The filter cake was soaked and washed with water (3.8 kg×3). The filter cake was soaked and washed with n-heptane (3.8 kg) twice. A sample was taken for wet cake IPC (HPLC result: Compound 1b ≥99.76%; impurity at RRT 1.08: 0.1057%; PMB-711: not detected; PMB-anisole: not detected; 711-int-3: not detected).

Drying: The filter cake was dried at 30~40° C. under vacuum for 24~72 h. A sample was taken for drying IPC (water content: 5.0%; ethanol: 271 ppm; EtOAc: 2 ppm; n-heptane: 10 ppm; toluene: 3 ppm; anisole, DMAC, and 1.4-dioxane: not detected).

Packed and weighed the production: Compound 1b (3.72 kg) was obtained as an off-white solid with a purity of 99.9% and in a quantitative yield (100.0%).

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. Crystalline Form A of Compound 1a represented by the formula: 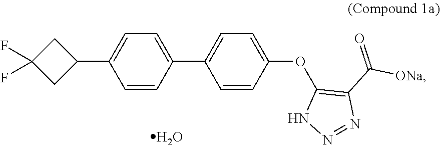
or a tautomer thereof, characterized by an X-ray powder diffraction (XRPD) pattern comprising peaks at 6.2, 9.2, 15.4, 28.0, and 29.3 degrees 2θ (each ±0.2 degrees 2θ).

2. The crystalline Form A of claim 1, wherein the X-ray powder diffraction pattern further comprises three or more peaks at 22.8, 24.9, 25.8, 27.3, 31.2, 31.7, and 34.4 degrees 2θ (each ±0.2 degrees 2θ).

3. The crystalline Form A of claim 1 or 2, wherein the X-ray powder diffraction pattern is substantially in accordance with FIG. 1.

4. The crystalline Form A of any one of claims 1 to 3, which is substantially free of one or more crystalline or amorphous forms of Compound 1 represented by the formula: 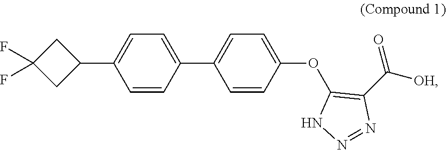 [SMILES: O=C(O)c1nn[nH]c1Oc1ccc(-c2ccc(C3CC(F)(F)C3)cc2)cc1]
a tautomer, a sodium salt, a hydrate, or a combination thereof, provided that the one or more crystalline or amorphous forms of Compound 1 are other than the crystalline Form A of Compound 1a.

5. The crystalline Form A of claim 4, which is substantially free of other crystalline or amorphous forms of Compound 1a.

6. The crystalline Form A of any one of claims 1 to 5, further characterized by a differential scanning calorimetry (DSC) thermogram comprising one or more endothermic peaks at about 118.5° C., about 240.8° C., and/or about 243.4° C.

7. The crystalline Form A of claim 6, wherein the differential scanning calorimetry (DSC) thermogram comprises an endothermic peak at about 118.5° C.

8. The crystalline Form A of claim 7, wherein the endothermic peak at about 118.5° C. has an onset temperature of about 89.6° C.

9. The crystalline Form A of any one of claims 6 to 8, wherein the differential scanning calorimetry (DSC) thermogram comprises an endothermic peak at about 240.81° C.

10. The crystalline Form A of claim 9, wherein the endothermic peak at about 240.81° C. has an onset temperature of about 238.1° C.

11. The crystalline Form A of any one of claims 6 to 10, wherein the differential scanning calorimetry (DSC) thermogram is substantially in accordance with FIG. 2.

12. The crystalline Form A of any one of claims 1 to 11, further characterized by a weight loss of from about 3% to about 5% upon heating to about 150° C., as measured by a thermal gravimetric analysis (TGA).

13. The crystalline Form A of claim 12, wherein the weight loss is about 4.2% upon heating from about 33.5° C. to about 150.0° C., as measured by the thermal gravimetric analysis (TGA).

14. The crystalline Form A of any one of claims 1 to 13, further characterized by a thermal gravimetric analysis (TGA) thermogram substantially in accordance with FIG. 3.

15. The crystalline Form A of any one of claims 1 to 14, further characterized by a polarized light microscopy (PLM) image substantially in accordance with FIG. 4.

16. The crystalline Form A of any one of claims 1 to 15, further characterized by: i) a weight gain of less than about 0.1% after undergoing a first dynamic vapor sorption cycle from about 10% relative humidity (RH) to about 95% RH at 25° C.; and/or ii) a weight loss of less than about 0.1% after undergoing a first dynamic vapor desorption cycle from about 95% relative humidity (RH) to about 10% RH at 25° C.

17. The crystalline Form A of claim 16, wherein a weight gain is less than about 0.03% after undergoing the first dynamic vapor desorption cycle from about 60% relative humidity (RH) to about 80% RH at 25° C.; and/or a weight loss is less than about 0.03% after undergoing the first dynamic vapor desorption cycle from about 80% relative humidity (RH) to about 60% RH at 25° C.

18. The crystalline Form A of any one of claims 1 to 17, having a dynamic vapor sorption profile substantially as shown in FIG. 5.

19. The crystalline Form A of any one of claims 1 to 18, wherein Compound 1a is in a monohydrate form and non-hygroscopic.

20. Crystalline Form D of Compound 1b represented by the formula: 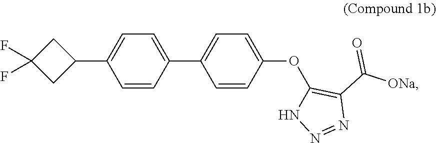
or a tautomer thereof, characterized by an X-ray powder diffraction (XRPD) pattern comprising peaks at 5.0, 10.0, 12.5, 17.6, and 22.7 degrees 2θ (each ±0.2 degrees 2θ).

21. The crystalline Form D of claim 20, wherein the X-ray powder diffraction pattern further comprises peaks at 7.5, 15.1, 20.1, 22.3, and 25.3 degrees 2θ (each ±0.2 degrees 2θ).

22. The crystalline Form D of claim 20 or 21, wherein the X-ray powder diffraction pattern is substantially in accordance with FIG. 6.

23. The crystalline Form D of any one of claims 20 to 22, which is substantially free of one or more crystalline or amorphous forms of Compound 1 represented by the formula: 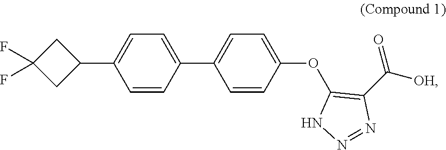 [SMILES: O=C(O)c1nn[nH]c1Oc1ccc(-c2ccc(C3CC(F)(F)C3)cc2)cc1]
a tautomer, a sodium salt, a hydrate, or a combination thereof, provided that the one or more crystalline or amorphous forms of Compound 1 are other than the crystalline Form D of Compound 1b.

24. The crystalline Form D of any one of claims 20 to 23, further characterized by a differential scanning calorimetry (DSC) thermogram comprising one or more endothermic peaks at about 241.2° C. and/or about 243.2° C.

25. The crystalline Form D of claim 24, wherein the differential scanning calorimetry (DSC) thermogram comprises an endothermic peak at about 241.2° C.

26. The crystalline Form D of claim 25, wherein the endothermic peak at about 241.2° C. has an onset temperature of about 238.4° C.

27. The crystalline Form D of any one of claims 24 to 26, wherein the DSC thermogram is substantially in accordance with FIG. 7.

28. The crystalline Form D of any one of claims 20 to 27, further characterized by a weight loss of less than about 1% upon heating to about 150° C., as measured by a thermal gravimetric analysis (TGA).

29. The crystalline Form D of claim 28, wherein the weight loss is about 0.6% upon heating from about 31.4° C. to about 150.0° C., as measured by the thermal gravimetric analysis (TGA).

30. The crystalline Form D of any one of claims 20 to 29, further characterized by a thermal gravimetric analysis (TGA) thermogram substantially in accordance with FIG. 8.

31. The crystalline Form D of any one of claims 20 to 30, further characterized by a polarized light microscopy (PLM) image substantially in accordance with FIG. 9.

32. The crystalline Form D of any one of claims 20 to 31, wherein Compound 1b is in an anhydrate form.

33. Crystalline Form E of Compound 1b represented by the formula: 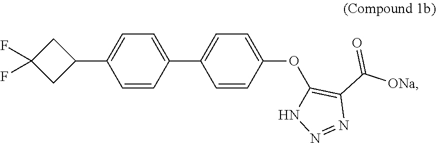
or a tautomer thereof and/or a hydrate thereof, characterized by an X-ray powder diffraction (XRPD) pattern comprising three or more peaks at 5.3, 12.7, 16.0, 19.6, 23.0, 25.1, and 28.9 degrees 2θ (each ±0.2 degrees 2θ).

34. The crystalline Form E of claim 33, wherein the X-ray powder diffraction pattern comprises a peak at 16.0 degrees 2θ (±0.2 degrees 2θ).

35. The crystalline Form E of claim 33 or 34, wherein the X-ray powder diffraction pattern is substantially in accordance with FIG. 10.

36. The crystalline Form E of any one of claims 33 to 35, which is substantially free of one or more crystalline or amorphous forms of Compound 1 represented by the formula: 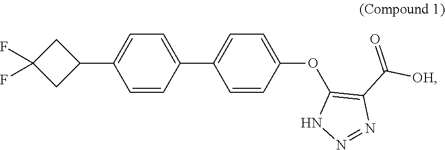 [SMILES: O=C(O)c1nn[nH]c1Oc1ccc(-c2ccc(C3CC(F)(F)C3)cc2)cc1]
a tautomer, a sodium salt, a hydrate, or a combination thereof, provided that the one or more crystalline or amorphous forms of Compound 1 are other than the crystalline Form E of Compound 1b.

37. The crystalline Form E of any one of claims 33 to 36, further characterized by a differential scanning calorimetry (DSC) thermogram comprising one or more endothermic peaks at about 64.5° C. and/or about 239.3° C.

38. The crystalline Form E of claim 37, wherein the differential scanning calorimetry (DSC) thermogram comprises an endothermic peak at about 239.3° C.

39. The crystalline Form E of claim 38, wherein the endothermic peak at about 239.3° C. has an onset temperature of about 232.5° C.

40. The crystalline Form E of any one of claims 37 to 39, wherein the DSC thermogram is substantially in accordance with FIG. 11.

41. The crystalline Form E of any one of claims 33 to 40, further characterized by a weight loss of from about 1.5% to about 3% upon heating to about 150° C., as measured by a thermal gravimetric analysis (TGA).

42. The crystalline Form E of claim 41, wherein the weight loss is about 2.1% upon heating from about 31.2° C. to about 150.0° C., as measured by the thermal gravimetric analysis (TGA).

43. The crystalline Form E of any one of claims 33 to 42, further characterized by a thermal gravimetric analysis (TGA) thermogram substantially in accordance with FIG. 12.

44. The crystalline Form E of any one of claims 33 to 43, wherein Compound 1b is in a hemihydrate form.

45. Co-crystalline Form C of Compound 1 represented by the formula: 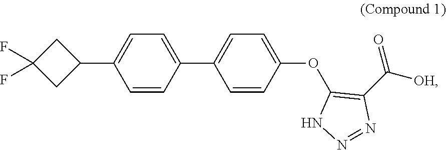 [SMILES: O=C(O)c1nn[nH]c1Oc1ccc(-c2ccc(C3CC(F)(F)C3)cc2)cc1]
and Compound 1b represented by the formula: 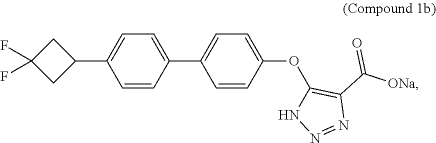
or a tautomer thereof, characterized by an X-ray powder diffraction (XRPD) pattern comprising peaks at 6.3, 16.0, 21.6, 25.9, and 28.8 degrees 2θ (each ±0.2 degrees 2θ).

46. The co-crystalline Form C of claim 45, wherein the X-ray powder diffraction pattern further comprises peaks at 18.7, 23.6, 24.4, 26.5, and 31.7 degrees 2θ (each ±0.2 degrees 2θ).

47. The co-crystalline Form C of claim 45 or 46, wherein the X-ray powder diffraction pattern further comprises peaks at 17.0, 19.8, 20.3, 33.9, and 34.1 degrees 2θ (each ±0.2 degrees 2θ).

48. The co-crystalline Form C of any one of claims 45 to 47, wherein the X-ray powder diffraction pattern is substantially in accordance with FIG. 13.

49. The co-crystalline Form C of any one of claims 45 to 48, which is substantially free of one or more crystalline or amorphous forms of Compound 1 represented by the formula: 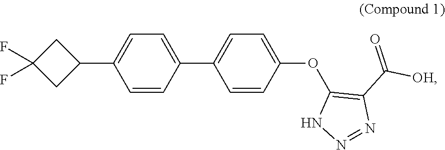 [SMILES: O=C(O)c1nn[nH]c1Oc1ccc(-c2ccc(C3CC(F)(F)C3)cc2)cc1]
a tautomer, a sodium salt, a hydrate, or a combination thereof, provided that the one or more crystalline or amorphous forms of Compound 1 are other than the co-crystalline Form C of Compound 1 and Compound 1b.

50. The co-crystalline Form C of any one of claims 45 to 49, further characterized by a differential scanning calorimetry (DSC) thermogram comprising one or more endothermic peaks at about 196.9° C. and/or about 215.1° C.

51. The co-crystalline Form C of claim 50, wherein the differential scanning calorimetry (DSC) thermogram comprises an endothermic peak at about 196.9° C.

52. The co-crystalline Form C of claim 51, wherein the endothermic peak at about 196.9° C. has an onset temperature of about 193.0° C.

53. The co-crystalline Form C of any one of claims 50 to 52, wherein the DSC thermogram is substantially in accordance with FIG. 14.

54. The co-crystalline Form C of any one of claims 45 to 53, further characterized by a weight loss of from about 10% to about 2% upon heating to about 150° C., as measured by a thermal gravimetric analysis (TGA).

55. The co-crystalline Form C of claim 54, wherein the weight loss is about 1.3% upon heating from about 30.0° C. to about 150.0° C., as measured by the thermal gravimetric analysis (TGA).

56. The co-crystalline Form C of any one of claims 45 to 55, further characterized by a thermal gravimetric analysis (TGA) thermogram substantially in accordance with FIG. 15.

57. The co-crystalline Form C of any one of claims 45 to 56, wherein Compound 1 and Compound 1b are each in an anhydrate form.

58. A pharmaceutical composition comprising crystalline Form A of Compound 1a represented by the formula: 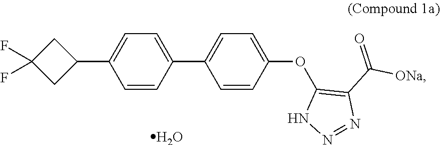
or a tautomer thereof, according to any one of claims 1 to 19, and a pharmaceutically acceptable excipient.

59. The pharmaceutical composition of claim 58, formulated for an oral administration.

60. The pharmaceutical composition of claim 58 or 59, in a tablet or a capsule.

61. A method for treating primary hyperoxaluria, type I (PH1) comprising administering to a subject in need thereof a therapeutically effective amount of Compound 1a or a tautomer thereof comprising crystalline Form A according to any one of claims 1 to 19 or a pharmaceutical composition according to claim 58.

62. The method of claim 61, the subject has kidney and/or bladder stones.

63. The method of claim 61 or 62, wherein Compound 1a or a tautomer thereof reduces glyoxylate level in the subject.

64. The method of any one of claims 61 to 63, wherein Compound 1a or a tautomer thereof reduces an accumulation of oxalate in kidney and/or urinary tract.

65. A method for treating kidney and/or bladder stones comprising administering to a subject in need thereof a therapeutically effective amount of Compound 1a or a tautomer thereof comprising crystalline Form A according to any one of claims 1 to 19 or a pharmaceutical composition according to claim 58.

66. The method of claim 65, wherein Compound 1a or a tautomer thereof reduces an accumulation of oxalate in kidney and/or urinary tract.

67. A method for inhibiting glycolate oxidase comprising contacting glycolate oxidase with an effective amount of Compound 1a or a tautomer thereof comprising crystalline Form A according to any one of claims 1 to 19 or a pharmaceutical composition according to claim 58.

68. The method of claim 67, wherein Compound 1a or a tautomer thereof reduces glyoxylate level.

69. A method of treating hyperoxaluria in a subject in need thereof, the method comprising administering to the subject a therapeutically effective amount of Compound 1a or a tautomer thereof comprising crystalline Form A according to any one of claims 1 to 19 or a pharmaceutical composition according to claim 58, wherein the therapeutically effective amount reduces a maximal urinary oxalate (uOxalate) excretion: i) to less than about 0.46 millimoles per 1.73 square meters per 24 hours (mmol/1.73 m²/24 hr)), or ii) by at least about 50% relative to a uOxalate excretion prior to the treating.

70. The method of claim 69, wherein the therapeutically effective amount is a total daily dosage of from about 5 mg to about 2,500 mg.

71. A method of treating hyperoxaluria in a subject in need thereof, the method comprising administering to the subject a therapeutically effective amount of Compound 1a or a tautomer thereof comprising crystalline Form A according to any one of claims 1 to 19 or a pharmaceutical composition according to claim 58, wherein the therapeutically effective amount is a total daily dosage of about 20 mg to 2,500 mg.

72. The method of any one of claims 69 to 71, wherein the hyperoxaluria is a primary hyperoxaluria.

73. The method of claim 72, wherein the primary hyperoxaluria is primary hyperoxaluria type 1 (PH1).

74. The method of any one of claims 69 to 73, wherein the hyperoxaluria is caused by overproduction of oxalate.

75. The method of any one of claims 69 to 74, wherein the subject has one or more mutations in an alanine-glyoxylate aminotransferase (AGXT) gene.

76. The method of any one of claims 69 to 75, wherein the subject has a uOxalate excretion of more than about 0.7 mmol/1.73 m² per 24 hours.

77. The method of any one of claims 69 to 76, wherein the subject has a plasma glycolate (pGlycolate) concentration of less than about 20 μmol/L.

78. The method of any one of claims 69 to 77, wherein the subject has an estimated glomerular filtration rate (eGFR) of at least about 30 mL/min/1.73 m².

79. The method of any one of claims 69 to 78, wherein the subject receives pyridoxine (Vitamin B6) at a stable dose for at least three (3) months prior to the treating and maintains the same dose during the treating.

80. The method of claim 79, wherein the subject has a plasma pyridoxine level within a normal range.

81. The method of any one of claims 69 to 80, wherein:
1) the subject is not a recipient of a liver transplant;
2) the subject does not receive a renal replacement therapy selected from the group consisting of hemodialysis, peritoneal dialysis, a continuous renal replacement therapy, or a combination thereof; and/or
3) the subject is not treated with a ribonucleic acid interference (RNAi) agent for at least nine (9) months prior to the treating.

82. The method of claim 81, wherein the RNAi agent is lumasiran.

83. The method of any one of claims 69 to 82, wherein the subject is human.

84. The method of claim 83, wherein the human is an adult of at least 18 years old or an adolescent of at least 12 years old with a body weight of at least 50 kg.

85. The method of claim 69, wherein the subject is a child from 6 to 12 years old or having a body weight of less than 50 kg.

86. The method of claim 69 or 71, wherein the primary hyperoxaluria is primary hyperoxaluria type 2 (PH2) or primary hyperoxaluria type 3 (PH3).

87. The method of claim 69 or 71, wherein the hyperoxaluria is not caused by a primary hyperoxaluria.

88. The method of any one of claims 69 to 87, wherein the therapeutically effective amount reduces the uOxalate excretion to less than about 0.46 mmol/1.73 m² per 24 hours.

89. The method of any one of claims 69 to 87, wherein the therapeutically effective amount reduces the uOxalate excretion by at least about 50% relative to a uOxalate excretion prior to the treating.

90. The method of any one of claims 69 to 89, wherein the therapeutically effective amount brings a ratio of uOxalate to urine creatinine (uCreatinine) to a normal range of less than about 63.7 mg/mg, which is 80 mmol/mmol.

91. The method of any one of claims 69 to 90, wherein the therapeutically effective amount reduces a plasma glycolate (pGlycolate) concentration in an absolute amount and/or in a relative amount compared to a pGlycolate concentration prior to the treating.

92. The method of any one of claims 69 to 91, wherein the therapeutically effective amount increases an estimated glomerular filtration rate (eGFR) in an absolute amount and/or in a relative amount compared to an eGFR prior to the treating.

93. A method of treating kidney stones in a subject in need thereof, the method comprising administering to the subject a therapeutically effective amount of Compound 1a or a tautomer thereof comprising crystalline Form A according to any one of claims 1 to 19 or a pharmaceutical composition according to claim 58, wherein the therapeutically effective amount is a total daily dosage of about 20 mg to 2,500 mg.

94. The method of claim 93, wherein the treating reduces a size of the kidney stones.

95. A method of preventing the development, worsening, or recurrence of kidney stones in a subject in need thereof, the method comprising administering to the subject a therapeutically effective amount of Compound 1a or a tautomer thereof comprising crystalline Form A according to any one of claims 1 to 19 or a pharmaceutical composition according to claim 58, wherein the therapeutically effective amount is a total daily dosage of about 20 mg to 2,500 mg.

96. The method of any one of claims 69 to 95, wherein the therapeutically effective amount is a total daily dosage of from about 50 mg to about 1000 mg, from about 75 mg to about 1000 mg, from about 75 mg to about 800 mg, or from about 75 mg to about 600 mg of Compound 1a, when the subject is an adult of at least 18 years old or an adolescent of at least years old with a body weight of at least 50 kg.

97. The method of claim 96, wherein the therapeutically effective amount is a total daily dosage of about 75 mg, about 300 mg, or about 600 mg of Compound 1a.

98. The method of claim 98, wherein the therapeutically effective amount is a total daily dosage of about 75 mg of Compound 1a.

99. The method of claim 98, wherein the therapeutically effective amount is a total daily dosage of about 300 mg of Compound 1a.

100. The method of claim 98, wherein said therapeutically effective amount is a total daily dosage of about 600 mg of compound 1a.

101. The method of any one of claims 69 to 95, wherein the therapeutically effective amount is adjusted to about ½, about ⅓, about ¼, or about ⅕ of a total daily dosage for the subject being an adult of at least 18 years old or an adolescent of at least 12 years old with a body weight of at least 50 kg.

102. The method of claim 69 to 101, wherein Compound 1a is administered orally.

103. The method of claim 69 to 102, wherein Compound 1a is administered once, twice, three times, or four times daily.

104. The method of claim 103, wherein said Compound 1a is administered once daily.

105. The method of any one of claims 69 to 104, wherein Compound 1a is administered chronically.

106. The method of any one of claims 69 to 104, wherein Compound 1a is administered for 28 days.

107. The method of any one of claims 69 to 104, wherein Compound 1a is administered for 12 months.

108. The method of any one of claims 69 to 104, wherein Compound 1a is administered for 36 months.

109. A method for preparing crystalline Form A of Compound 1a represented by the formula: 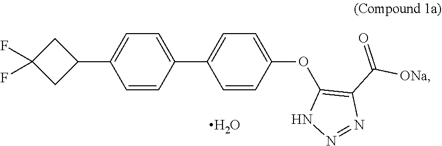
according to any one of claims 1 to 19, comprising:
a) providing a first mixture comprising:
i) Compound 1 represented by the formula: 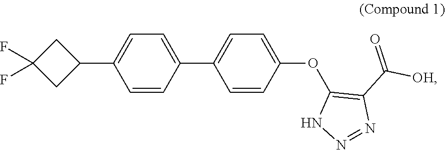 [SMILES: O=C(O)c1nn[nH]c1Oc1ccc(-c2ccc(C3CC(F)(F)C3)cc2)cc1]
a tautomer, a sodium salt, a hydrate, or a combination thereof; and
ii) a solvent mixture comprising tetrahydrofuran (THF) and water;
b) stirring the first mixture at a temperature of from about 45° C. to about 60° C. to form a second mixture;
c) filtering the second mixture of step b) to provide a third mixture comprising Compound 1b represented by the formula: 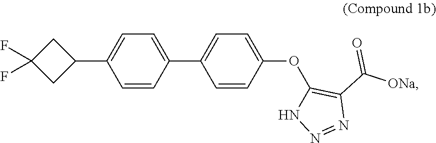
or a tautomer thereof;
d) cooling the third mixture to a temperature of less than 10° C. to form a slurry; and
e) isolating a solid from the slurry to provide the crystalline Form A.

110. The method of claim 109, wherein, prior to step d), one or more seeds of crystalline Form A of Compound 1a are added to the third mixture of step c).

111. The method of claim 109 or 110, wherein, in step a), the solvent mixture has a THF:H₂O ratio of about 1:3 by volume.

112. The method of any one of claims 109 to 111, wherein, in step a), Compound 1, a tautomer, a sodium salt, a hydrate, or a combination thereof is a mixture of Compound 1a, Compound 1b, and Compound 1.

113. The method of claim 112, wherein, in step a), the mixture of Compound 1a, Compound 1b, and Compound 1 is present in the first mixture in a concentration of from about 25 mg/mL to about 50 mg/mL or about 33 mg/mL.

114. The method of any one of claims 109 to 113, wherein step a) is conducted at room temperature; and/or step b) is conducted at a temperature of about 50° C.

115. The method of any one of claims 109 to 114, wherein step b) is conducted for a period of from about 1 to about 5 hours.

116. The method of claim 114 or 115, wherein step b) is conducted at a temperature of about 50° C. for a period of about 3 hours.

117. The method of any one of claims 109 to 116, wherein, in step c), the third mixture is a solution comprising Compound 1b or a tautomer thereof.

118. The method of any one of claims 109 to 117, wherein step d) is conducted at a temperature of about 5° C.; and the slurry is cooled at the temperature of about 5° C. for a period of from about 2 to 3 days.

119. The method of any one of claims 109 to 118, wherein step e) is conducted by filtration.

120. The method of any one of claims 109 to 119, wherein, in step e), the solid is further dried under a vacuum at room temperature to provide the crystalline Form A.

121. A method for preparing crystalline Form A of Compound 1a according to any one of claims 1 to 19, comprising:
a) at room temperature, providing a first mixture comprising i) a mixture of Compound 1a, Compound 1b, and Compound 1, each of which is represented by the formula: 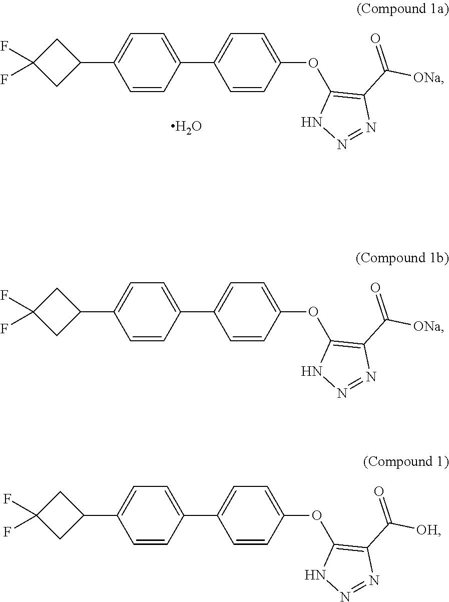
or a tautomer thereof; and ii) a solvent mixture comprising tetrahydrofuran (THF) and water in a THF:H₂O ratio of about 1:3 by volume;
b) stirring the first mixture at a temperature of about 50° C. to form a second mixture;
c) filtering the second mixture of step b) to provide a third mixture comprising Compound 1b or a tautomer thereof; and one or more seeds of crystalline Form A of Compound 1a are added to the third mixture;
d) cooling the third mixture to a temperature of about 5° C. to form a slurry; and
e) isolating a solid from the slurry to provide the crystalline Form A.

122. The method of claim 121, wherein:
in step a), the mixture of Compound 1a, Compound 1b, and Compound 1 is present in the first mixture in a concentration of about 33 mg/mL;
step b) is conducted for a period of about 3 hours;
in step c), the third mixture is a solution;
in step d), the slurry is cooled at the temperature of about 5° C. for a period of about 2.5 days; and
step e) is conducted by filtration and the solid is further dried under a vacuum at room temperature to provide the crystalline Form A.

123. A process for preparing a compound represented by formula (I): 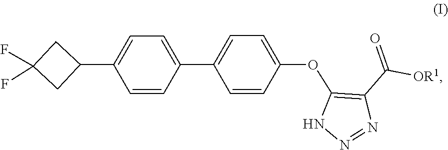
or a tautomer and/or a salt thereof, comprising:
a) combining a compound of formula (IV): 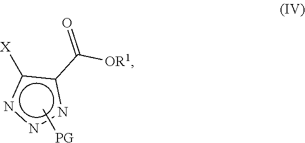
or a salt thereof, a compound of formula (III): 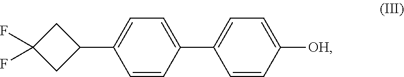 [SMILES: Oc1ccc(-c2ccc(C3CC(F)(F)C3)cc2)cc1]
or a salt thereof, and a first base in a first solvent to form a compound of formula (II): 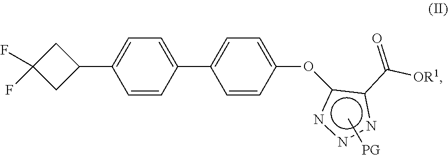
or a salt thereof; and
b) removing the PG group of the compound of formula (II) or the salt thereof to provide a compound of formula (I): 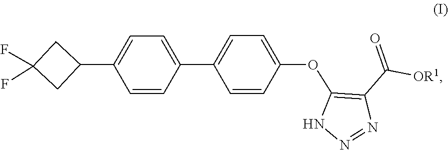
or a tautomer an or a salt thereof,
wherein:
R¹ is C₁₋₆ alkyl;
X in formula (IV) is halogen or a sulfonate; 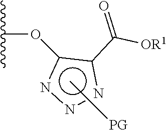
in formula (IV) or (II) is represented by the formula: 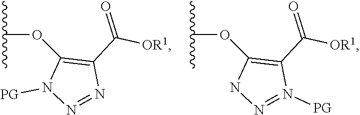
or a mixture thereof; and
PG is an amine-protecting group.

124. The process of claim 123, wherein R¹ is ethyl.

125. The process of claim 123 or 124, wherein X in formula (IV) is Cl, Br, I, OMs, OTs, or OTf.

126. The process of claim 125, wherein X in formula (IV) is Cl.

127. The process of any one of claims 123 to 126, wherein the amine-protecting group is [2-(trimethylsilyl)ethoxy]methyl, 4-methoxybenzyl, or 2,4-dimethoxybenzyl.

128. The process of claim 127, wherein the amine-protecting group is 4-methoxybenzyl.

129. The process of any one of claims 123 to 128, wherein the compound of formula (IV) is represented by formula (IVa):

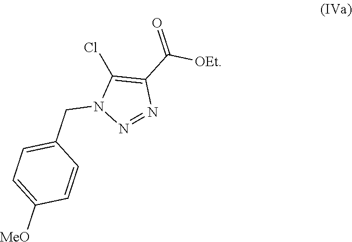 [SMILES: CCOC(=O)c1nnn(Cc2ccc(OC)cc2)c1Cl]

130. The process of any one of claims 123 to 129, wherein the compound of formula (II) is represented by formula (IIa):

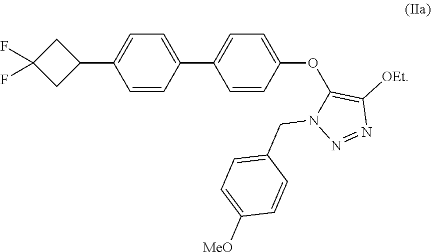 [SMILES: CCOc1nnn(Cc2ccc(OC)cc2)c1Oc1ccc(-c2ccc(C3CC(F)(F)C3)cc2)cc1]

131. The process of any one of claims 123 to 131, wherein, in step a), the first base is:
i) an alkali base selected from the group consisting of sodium carbonate, potassium carbonate, cesium carbonate, sodium phosphate tribasic, potassium phosphate tribasic, sodium acetate, potassium acetate, and cesium acetate;
ii) a tertiary amine selected from the group consisting of triethylamine, tri-n-butylamine, N,N-diisopropylethylamine, N-methylpyrrolidine, N-methylmorpholine, dimethylaniline, diethylaniline, 1,8-bis(dimethylamino)naphthalene, quinuclidine, and 1,4-diazabicylo[2.2.2]-octane (DABCO);
iii) an aromatic amine base selected from the group consisting of pyridine, lutidines, collidines, 4-dimethylaminopyridine, imidazole, dimethylaniline, and diethylaniline;
iv) an amidine-based compound selected from the group consisting of 1,8-diazabicyclo[5.4.0]undec-7-ene (DBU) and 1,5-diazabicyclo[4.3.0]non-5-en (DBN); or
v) a combination thereof.

132. The process of claim 131, wherein, in step a), the first base comprises 1,8-diazabicyclo[5.4.0]undec-7-ene (DBU).

133. The process of claim 131, wherein, in step a), the first base comprises cesium carbonate.

134. The process of any one of claims 123 to 133, wherein, in step a), the compound of formula (III) is in an amount of from 1.0 to 1.5 equivalents, from about 1.0 to about 1.4 equivalents, from about 1.0 to about 1.3 equivalents, from about 1.0 to about 1.2 equivalents, from about 1.0 to about 1.1 equivalents, or about 1.0 equivalents, relative to the compound of formula (IV).

135. The process of any one of claims 123 to 134, wherein, in step a), the first base is in an amount of from about 1.0 to about 2.0 equivalents, from about 1.0 to about 1.5 equivalents, or about 1.35 equivalents, relative to the compound of formula (IV).

136. The process of claim 134 or 135, wherein, in step a), the compound of formula (III) is in an amount of about 1.0 equivalents and the first base is in an amount of about 1.35 equivalents, relative to the compound of formula (IV).

137. The process of any one of claims 123 to 133, wherein, in step a), the first solvent is benzene, toluene, dioxane, acetone, ethyl acetate (EtOAc), tetrahydrofuran (THF), 2-methyl-tetrahydrofuran (MeTHF), acetonitrile (ACN), N-methylpyrrolidone (NMP), N,N-dimethylformamide (DMF), N,N-dimethylacetamide (DMAC), dimethoxyethane (DME), dimethyl sulfoxide (DMSO), propylene carbonate (PC), or a combination thereof.

138. The process of claim 137, wherein the first solvent is N,N-dimethylacetamide (DMAC) or N-methylpyrrolidone (NMP).

139. The process of claim 137, wherein, in step a), first base comprises 1,8-diazabicyclo[5.4.0]undec-7-ene (DBU) and the first solvent comprises N,N-dimethylacetamide (DMAC); or the first base comprises cesium carbonate and the first solvent comprises N-methylpyrrolidone (NMP).

140. The process of any one of 123 to 139, wherein step a) is conducted at a temperature of from about 70° C. to about 110° C., from about 80° C. to about 100° C., or from about 85° C. to about 90° C.

141. The process of claim 140, wherein step a) is conducted at a temperature of from about 85° C. to about 90° C.

142. The process of any one of claims 123 to 141, wherein, in step b), the PG group is removed by treating with a first acid in a second solvent.

143. The process of claim 142, wherein the first acid is trifluoroacetic acid.

144. The process of claim 142 or 143, wherein the second solvent comprises anisole.

145. The process of any one of claims 123 to 144, further comprising:
c) combining the compound of formula (I), or the tautomer and/or the salt thereof, with a second base in a third solvent; and
d) acidifying with a second acid to provide Compound 1 represented by the formula: 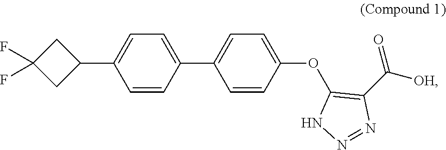 [SMILES: O=C(O)c1nn[nH]c1Oc1ccc(-c2ccc(C3CC(F)(F)C3)cc2)cc1]
a tautomer, a salt, a hydrate, or a combination thereof.

146. The process of claim 145, wherein the second base is lithium hydroxide, sodium hydroxide, or potassium hydroxide.

147. The process of claim 146, wherein the second base is sodium hydroxide.

148. The process of any one of claims 145 to 147, wherein the third solvent is water, C₁₋₄ alcohol, dioxane, tetrahydrofuran (THF), 2-methyl-tetrahydrofuran (MeTHF), acetonitrile (ACN), dimethoxyethane (DME), or a combination thereof.

149. The process of claim 148, wherein the third solvent comprises water.

150. The process of any one of claims 145 to 149, wherein the second acid is HCl.

151. The process of any one of claims 145 to 150, wherein steps c) and d) are each conducted in an aqueous solution.

152. The process of any one of claims 123 to 151, wherein the compound of formula (III) is prepared by:
combining 1-bromo-4-(3,3-difluorocyclobutyl)benzene represented by the formula: 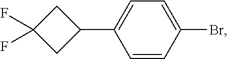 [SMILES: FC1(F)CC(c2ccc(Br)cc2)C1]
or a salt thereof, (4-hydroxyphenyl)boronic acid represented by the formula: 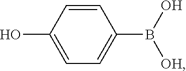
a palladium catalyst, and a third base in a fourth solvent to provide the compound of formula (III): 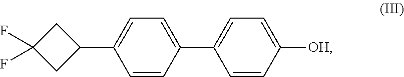 [SMILES: Oc1ccc(-c2ccc(C3CC(F)(F)C3)cc2)cc1]
or a salt thereof.

153. The process of claim 152, wherein the palladium catalyst is Pd(dppf)Cl₂, Pd(dppf)Cl₂·CH₂Cl₂, or Pd(dtbpf)Cl₂.

154. The process of claim 152 or 153, wherein the third base is sodium carbonate.

155. The process of any one of claims 152 to 154, wherein the fourth solvent comprises 1,4-dioxane.

156. A process for preparing Compound 1 represented by the formula: 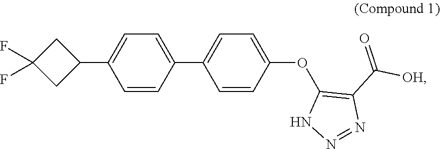 [SMILES: O=C(O)c1nn[nH]c1Oc1ccc(-c2ccc(C3CC(F)(F)C3)cc2)cc1]
a tautomer, a sodium salt, a hydrate, or a combination thereof, comprising:
a) combining a compound of formula (IVa): 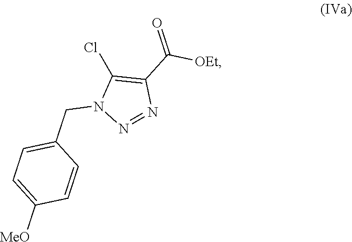 [SMILES: CCOC(=O)c1nnn(Cc2ccc(OC)cc2)c1Cl]
or a salt thereof, a compound of formula (III): 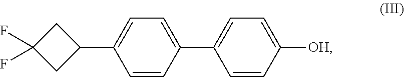 [SMILES: Oc1ccc(-c2ccc(C3CC(F)(F)C3)cc2)cc1]
or a salt thereof, and a first base in a first solvent to form a compound of formula (IIa): 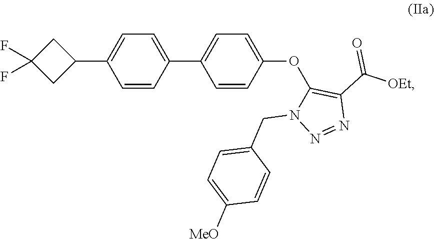 [SMILES: CCOC(=O)c1nnn(Cc2ccc(OC)cc2)c1Oc1ccc(-c2ccc(C3CC(F)(F)C3)cc2)cc1]
or a salt thereof, wherein the first base is 1,8-diazabicyclo[5.4.0]undec-7-ene (DBU) and the first solvent comprises N,N-dimethylacetamide (DMAC); or the first base is cesium carbonate and the first solvent comprises N-methylpyrrolidone (NMP);
b) treating the compound of formula (IIa) or the salt thereof, with trifluoroacetic acid and anisole to provide a compound of formula (Ia-1): 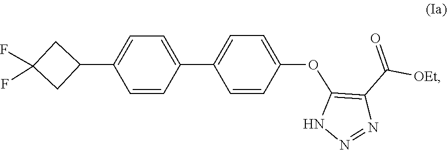 [SMILES: CCOC(=O)c1nn[nH]c1Oc1ccc(-c2ccc(C3CC(F)(F)C3)cc2)cc1]
or a tautomer and/or a salt thereof;
c) saponifying the compound of formula (Ia), or the tautomer and/or the salt thereof, with an aqueous solution of sodium hydroxide; and
d) acidifying with an aqueous solution of HCl to provide Compound 1, the tautomer, the sodium salt, the hydrate, or the combination thereof.

157. The process of claim 156, wherein, in step a), first base is 1,8-diazabicyclo[5.4.0]undec-7-ene (DBU) and the first solvent is N,N-dimethylacetamide (DMAC).

158. The process of claim 156 or 157, wherein, in step a), the compound of formula (III) is in an amount of about 1.0 equivalents and DBU is in an amount of about 1.35 equivalents, relative to the compound of formula (IVa).

159. The process of any one of claims 156 to 158, wherein step a) is conducted at a temperature of from about 85° C. to about 90° C.

160. The process of any one of claims 156 to 159, wherein step d) is conducted by acidifying a filtrate of a reaction mixture of step c).

161. The process of claim 160, wherein step d) is conducted according to steps:
d1) adding an aqueous 6M HCl solution to form a first slurry having a pH value of from about 9 to about 10;
d1) adding an aqueous 2M HCl solution to the first slurry to form a second slurry having a pH value of from about 7 to about 8;
d3) mixing the second slurry; and
d4) isolating a solid from the second slurry to provide Compound 1, substantially in a mono-sodium salt form represented by formula (Ib): 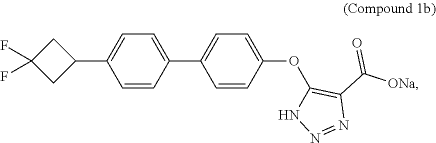
or a tautomer thereof.

162. A compound, represented by formula (III): 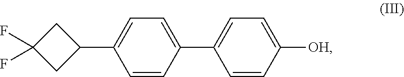 [SMILES: Oc1ccc(-c2ccc(C3CC(F)(F)C3)cc2)cc1]
or a salt thereof.
